(19) United States
(12) Patent Application Publication
(10) Pub. No.: US 2014/0065097 A1
(43) Pub. Date: Mar. 6, 2014

(54) Immunotherapy

(71) Applicant: Roche Glycart AG, Schlieren (CH)

(72) Inventors: Christian Gerdes, Erlenbach (CH); Christian Klein, Bonstetten (CH); Ekkenhard Moessner, Kreuzlingen (CH); Valeria G. Nicolini, Erlenbach (CH); Pablo Umana, Wollerau (CH)

(73) Assignee: Roche Glycart AG, Schlieren (CH)

(21) Appl. No.: 13/971,757

(22) Filed: Aug. 20, 2013

Related U.S. Application Data
(63) Continuation of application No. 13/367,881, filed on Feb. 7, 2012, now abandoned.

(30) Foreign Application Priority Data
Feb. 10, 2011 (EP) 11153976.3

(51) Int. Cl. C07K 16/32; A61K 47/48

(52) U.S. Cl. CPC C07K 16/32; A61K 47/48723
USPC 424/85.2; 424/134.1; 424/85.7; 424/85.1

(57) ABSTRACT

The present invention provides combinations of (a) an immunoconjugate comprising at least one antigen-binding moiety and an effector moiety, and (b) an antibody engineered to have increased effector function, for use in treating a disease in an individual in need thereof. Further provided are pharmaceutical compositions comprising the combinations, and methods of using them.

### RELATED APPLICATIONS

This application is a continuation of U.S. utility application Ser. No. 13/367,881 which claims the benefit of European Patent Application No. EP 11153976.3, filed Feb. 10, 2011. The contents of these Applications are hereby incorporated by reference in their entirety.

### SEQUENCE LISTING

This application contains a Sequence Listing which has been submitted via EFS-Web and is hereby incorporated by reference in its entirety. Said TXT copy, created on Aug. 20, 2013, is named P4821C1_Sequence_Listing.txt, is 273,224 bytes in size, and is IBM PC/XT/AT compatible.

### FIELD OF THE INVENTION

The present invention generally relates to immunotherapy. More particularly, the invention concerns antigen-targeted immunoconjugates and Fc-engineered antibodies for combined use as immunotherapeutic agents. In addition, the invention relates to pharmaceutical compositions comprising combinations of said immunoconjugates and antibodies and methods of using the same in the treatment of disease.

### BACKGROUND

The selective destruction of an individual cell or a specific cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. Effector cells can be activated by various stimuli, including a number of cytokines that induce signaling events through binding to their receptors on the surface of immune cells. For example interleukin-2 (IL-2), which, inter alia, stimulates proliferation and activation of cytotoxic T cells and NK cells, has been approved for the treatment of metastatic renal cell carcinoma and malignant melanoma. However, rapid blood clearance and lack of tumor specificity require systemic administration of high doses of a cytokine in order to achieve a sufficiently high concentration of the cytokine at the tumor site to activate an immune response or have an anti-tumor effect. These high systemic levels of cytokine can lead to severe toxicity and adverse reactions, as is the case also for IL-2. For use in cancer therapy, it is therefore desirable to specifically deliver cytokines to the tumor or tumor microenvironment. This can be achieved by conjugating the cytokine to a targeting moiety, e.g. an antibody or an antibody fragment, specific for a tumor antigen. A further advantage of such immunoconjugates is their increased serum half-life compared to the unconjugated cytokine Their ability to maximize immunostimulatory activities at the site of a tumor whilst keeping systemic side effects to a minimum at a lower dose makes cytokine immunoconjugates optimal immunotherapeutic agents.

Another way of activating effector cells is through the engagement of activating Fc receptors on their surface by the Fc portion of immunoglobulins or recombinant fusion proteins comprising an Fc region. The so-called effector functions of an antibody which are mediated by its Fc region are an important mechanism of action in antibody-based cancer immunotherapy. Antibody-dependent cell-mediated cytotoxicity, the destruction of antibody-coated target cells (e.g. tumor cells) by NK cells, is triggered when antibody bound to the surface of a cell interacts with Fc receptors on the NK cell. NK cells express FcγRIIIa (CD16a) which recognizes immunoglobulins of the IgG1 or IgG3 subclass. Further effector functions include antibody-dependent cell-mediated phagocytosis (ADCP) and complement dependent cytotoxicity (CDC), and vary with the class and subclass of the antibody since different immune cell types bear different sets of Fc receptors which recognize different types and subtypes of immunoglobulin heavy chain constant domains (e.g. α, δ, γ, ε, or μ heavy chain constant domains, corresponding to IgA, IgD, IgE, IgG, or IgM class antibodies, respectively). Various strategies have been employed to increase the effector functions of antibodies. For example, Shields et al. (J Biol Chem 9(2), 6591-6604 (2001)) show that amino acid substitutions at positions 298, 333, and/or 334 of the Fc region (EU numbering of residues) improve the binding of antibodies to FcγIIIa receptor and ADCC. Further antibody variants having amino acid modifications in the Fc region and exhibiting improved Fc receptor binding and effector function are described e.g. in U.S. Pat. No. 6,737,056, WO 2004/063351 and WO 2004/099249. Alternatively, increased Fc receptor binding and effector function can be obtained by altering the glycosylation of an antibody. IgG1 type antibodies, the most commonly used antibodies in cancer immunotherapy, have a conserved N-linked glycosylation site at Asn 297 in each CH2 domain of the Fc region. The two complex biantennary oligosaccharides attached to Asn 297 are buried between the CH2 domains, forming extensive contacts with the polypeptide backbone, and their presence is essential for the antibody to mediate effector functions including antibody-dependent cell-mediated cytotoxicity (ADCC) (Lifely et al., Glycobiology 5, 813-822 (1995); Jefferis et al., Immunol Rev 163, 59-76 (1998); Wright and Morrison, Trends Biotechnol 15, 26-32 (1997)). Protein engineering studies have shown that FcγRs interact with the lower hinge region of the IgG CH2 domain (Lund et al., J Immunol 157, 4963-69 (1996)). However, FcγR binding also requires the presence of the oligosaccharides in the CH2 region (Lund et al., J Immunol 157, 4963-69 (1996); Wright and Morrison, Trends Biotech 15, 26-31 (1997)), suggesting that either oligosaccharide and polypeptide both directly contribute to the interaction site or that the oligosaccharide is required to maintain an active CH2 polypeptide conformation. Modification of the oligosaccharide structure can therefore be explored as a means to increase the affinity of the interaction between IgG1 and FcγR, and to increase ADCC activity of IgG1 antibodies. Umaña et al. (Nat Biotechnol 17, 176-180 (1999) and U.S. Pat. No. 6,602,684 (WO 99/54342), the contents of which are hereby incorporated by reference in their entirety) showed that overexpression of β(1,4)-N-acetylglucosaminyltransferase III (GnTIII), a glycosyltransferase catalyzing the formation of bisected oligosaccharides, in Chinese hamster ovary (CHO) cells significantly increases the in vitro ADCC activity of antibodies produced in those cells. Overexpression of GnTIII in production cell lines leads to antibodies enriched in bisected oligosaccharides, which are generally also non-fucosylated and of the hybrid type. If in addition to GnTIII, mannosidase II (ManII) is overexpressed in production cell lines, antibodies enriched in bisected, non-fucosylated oligosaccharides of the complex type are obtained (Ferrara et al., Biotechn Bioeng 93, 851-861 (2006)). Both types of antibodies show strongly increased ADCC, as compared to antibodies with unmodified glycans, but only antibodies in which the majority of the N-glycans are of the complex type are able to induce significant complement-dependent cytotoxicity (Ferrara et al., Biotechn Bioeng 93, 851-861 (2006)). The critical factor for the increase of ADCC activity appears to be the elimination of fucose from the innermost N-acetylglucosamine residue of the oligosaccharide core, which improves binding of the IgG Fc domain to FcγRIIIa (Shinkawa et al., J Biol Chem 278, 3466-3473 (2003)). Further methods for producing antibodies with reduced fucosylation include, e.g. expression in α(1,6)-fucosyltransferase deficient host cells (Yamane-Ohnuki et al., Biotech Bioeng 87, 614-622 (2004); Niwa et al., J Immunol Methods 306, 151-160 (2006)).

Despite the successes achieved in anti-cancer immunotherapy by the use of free cytokines, immunoconjugates or engineered antibodies, there is a continuous need for novel efficacious and safe treatment options in cancer therapy.

### SUMMARY OF THE INVENTION

The present inventors have found that the combination of these two strategies for local immune cell activation, i.e. simultaneous stimulation of effector cells by cytokine immunoconjugates and by antibodies engineered to have increased effector functions, greatly improves the efficacy of anti-cancer immunotherapy. Accordingly, the present invention provides a combination of (a) an immunoconjugate comprising at least one antigen-binding moiety and an effector moiety, and (b) an antibody engineered to have increased effector function, for use in treating a disease in an individual in need thereof. The present invention also provides for a method of stimulating effector cell function in an individual comprising administering to the individual in need thereof an effective amount of (a) an immunoconjugate comprising at least one antigen-binding moiety and an effector moiety, and (b) an antibody engineered to have increased effector function. Further, the present invention also provides for a method of treating cancer in an individual comprising administering to the individual in need thereof a therapeutically effective amount of (a) an immunoconjugate comprising at least one antigen-binding moiety and an effector moiety, and (b) an antibody engineered to have increased effector function. In one embodiment the effector moiety is a cytokine. In one embodiment the cytokine is selected from the group consisting of IL-2, GM-CSF, IFN-α, and IL-12. In a particular embodiment the effector moiety is IL-2. In another embodiment the effector moiety is IL-12. In another particular embodiment the IL-2 effector moiety is a mutant IL-2 effector moiety comprising at least one amino acid mutation, particularly an amino acid substitution, that reduces or abolishes the affinity of the mutant IL-2 effector moiety to the α-subunit of the IL-2 receptor but preserves the affinity of the mutant IL-2 effector moiety to the intermediate-affinity IL-2 receptor, compared to the non-mutated IL-2 effector moiety. In a specific embodiment, the mutant IL-2 effector moiety comprises one, two or three amino acid substitutions at one, two or three position(s) selected from the positions corresponding to residue 42, 45, and 72 of human IL-2. In a more specific embodiment, the mutant IL-2 effector moiety comprises three amino acid substitutions at the positions corresponding to residue 42, 45 and 72 of human IL-2. In an even more specific embodiment, the mutant IL-2 effector moiety is human IL-2 comprising the amino acid substitutions F42A, Y45A and L72G. In certain embodiments the mutant IL-2 effector moiety additionally comprises an amino acid mutation at a position corresponding to position 3 of human IL-2, which eliminates the O-glycosylation site of IL-2. In a specific embodiment the mutant IL-2 effector moiety comprises the amino acid sequence of SEQ ID NO: 2. In one embodiment the effector moiety is a single-chain effector moiety.

In one embodiment the antigen-binding moiety is an antibody or an antibody fragment. In one embodiment the effector moiety shares an amino- or carboxy-terminal peptide bond with the antigen-binding moiety. In one embodiment the antigen-binding moiety is selected from a Fab molecule and a scFv molecule. In one embodiment the antigen-binding moiety is a Fab molecule. In another embodiment the antigen-binding moiety is a scFv molecule. In one embodiment the immunoconjugate comprises a first and a second antigen-binding moiety. In one embodiment the first and the second antigen-binding moieties are independently selected from a Fab molecule and a scFv molecule. In one embodiment each of the first and the second antigen-binding moieties is a Fab molecule, wherein the Fab molecule comprises a heavy and light chain. In another embodiment each of the first and the second antigen-binding moieties is a scFv molecule. In one embodiment the effector moiety shares an amino- or carboxy-terminal peptide bond with the first antigen-binding moiety, and the second antigen-binding moiety shares an amino- or carboxy-terminal peptide bond with either the effector moiety or the first antigen-binding moiety. In one embodiment the effector moiety shares an amino-terminal peptide bond with the first antigen-binding moiety and a carboxy-terminal peptide bond with the second antigen-binding moiety. In one embodiment the immunoconjugate essentially consists of an effector moiety and a first and a second antigen-binding moiety joined by one or more linker sequences. In a specific embodiment the immunoconjugate comprises an effector moiety, particularly a single chain effector moiety, and a first and a second Fab molecule, wherein the effector moiety is joined at its amino-terminal amino acid to the carboxy-terminus of the heavy or light chain of the first Fab molecule, and wherein the effector moiety is joined at its carboxy-terminal amino acid to the amino-terminus of the heavy or light chain of the second Fab molecule.

In certain embodiments the antigen-binding moiety is directed to an antigen presented on a tumor cell or in a tumor cell environment. In a specific embodiment the antigen-binding moiety is directed to an antigen selected from the group of Fibroblast Activation Protein (FAP), the A1 domain of Tenascin-C (TNC A1), the A2 domain of Tenascin-C (TNC A2), the Extra Domain B of Fibronectin (EDB), Carcinoembryonic Antigen (CEA) and Melanoma-associated Chondroitin Sulfate Proteoglycan (MCSP).

In one embodiment the increased effector function is selected from the group of increased binding to an activating Fc receptor, increased ADCC, increased ADCP, increased CDC, increased and increased cytokine secretion. In one embodiment the increased effector function is increased binding to an activating Fc receptor. In a specific embodiment the activating Fc receptor is selected from the group of FcγRIIIa, FcγRI, and FcRγIIa. In one embodiment the activating Fc receptor is FcγRIIIa. In one embodiment the increased effector function is increased ADCC. In one embodiment the increased effector function is increased binding to an activating Fc receptor and increased ADCC.

In one embodiment the antibody is engineered by introduction of one or more amino acid mutations in the Fc region. In a specific embodiment the amino acid mutations are amino acid substitutions. In one embodiment the antibody is engineered by modification of the glycosylation in the Fc region. In a specific embodiment the modification of the glycosylation in the Fc region is an increased proportion of non-fucosylated oligosaccharides in the Fc region, as compared to a non-engineered antibody. In an even more specific embodiment the increased proportion of non-fucosylated oligosaccharides in the Fc region is at least 20%, preferably at least 50%, most preferably at least 70% of non-fucosylated oligosaccharides in the Fc region. In another specific embodiment the modification of the glycosylation in the Fc region is an increased proportion of bisected oligosaccharides in the Fc region, as compared to a non-engineered antibody. In an even more specific embodiment the increased proportion of bisected oligosaccharides in the Fc region is at least about 20%, preferably at least 50%, and most preferably at least 70% of bisected oligosaccharides in the Fc region. In yet another specific embodiment the modification of the glycosylation in the Fc region is an increased proportion of bisected, non-fucosylated oligosaccharides in the Fc region, as compared to a non-engineered antibody. Preferably the antibody has at least about 25%, at least about 35%, or at least about 50% of bisected, non-fucosylated oligosaccharides in the Fc region. In a particular embodiment the antibody is engineered to have an increased proportion of non-fucosylated oligosaccharides in the Fc region as compared to a non-engineered antibody. An increased proportion of non-fucosylated oligosaccharides in the Fc region of an antibody results in the antibody having increased effector function, in particular increased ADCC. In a particular embodiment the non-fucosylated oligosaccharides are bisected, non-fucosylated oligosaccharides.

In one embodiment the antibody is a full-length IgG class antibody, particularly an IgG1 subclass antibody. In certain embodiments the antibody is directed to an antigen presented on a tumor cell. In a specific embodiment the antibody is directed to an antigen selected from the group of CD20, Epidermal Growth Factor Receptor (EGFR), HER2, HER3, Insulin-like Growth Factor 1 Receptor (IGF-1R), c-Met, CUB domain-containing protein-1 (CDCP1), Carcinoembryonic Antigen (CEA) and Melanoma-associated Chondroitin Sulfate Proteoglycan (MCSP).

In a particular embodiment the antibody is an anti-CD20 antibody engineered to have an increased proportion of non-fucosylated oligosaccharides in the Fc region as compared to a non-engineered antibody. Suitable anti-CD20 antibodies are described in WO 2005/044859, which is incorporated herein by reference in its entirety. In another particular embodiment the antibody is an anti-EGFR antibody engineered to have an increased proportion of non-fucosylated oligosaccharides in the Fc region as compared to a non-engineered antibody. Suitable anti-EGFR antibodies are described in WO 2006/082515 and WO 2008/017963, each of which is incorporated herein by reference in its entirety. In a further particular embodiment the antibody is an anti-IGF-1R antibody engineered to have an increased proportion of non-fucosylated oligosaccharides in the Fc region as compared to a non-engineered antibody. Suitable anti-IGF-1R antibodies are described in WO 2008/077546, which is incorporated herein by reference in its entirety. In yet another particular embodiment the antibody is an anti-CEA antibody engineered to have an increased proportion of non-fucosylated oligosaccharides in the Fc region as compared to a non-engineered antibody. Suitable anti-CEA antibodies are described in PCT publication number WO 2011/023787, which is incorporated herein by reference in its entirety. In yet another particular embodiment the antibody is an anti-HER3 antibody engineered to have an increased proportion of non-fucosylated oligosaccharides in the Fc region as compared to a non-engineered antibody. Suitable anti-HER3 antibodies are described in PCT publication number WO 2011/076683, which is incorporated herein by reference in its entirety. In yet another particular embodiment the antibody is an anti-CDCP1 antibody engineered to have an increased proportion of non-fucosylated oligosaccharides in the Fc region as compared to a non-engineered antibody. Suitable anti-CDCP1 antibodies are described in PCT publication number WO 2011/023389, which is incorporated herein by reference in its entirety. In one embodiment the antibody is engineered to have modified glycosylation in the Fc region, as compared to a non-engineered antibody, by producing the antibody in a host cell having altered activity of one or more glycosyltransferase.

In one embodiment the antibody is engineered to have an increased proportion of non-fucosylated oligosaccharides in the Fc region, as compared to a non-engineered antibody, by producing the antibody in a host cell having increased β(1,4)-N-acetylglucosaminyltransferase III (GnTIII) activity. In a particular embodiment the host cell additionally has increased α-mannosidase II (ManII) activity. In another embodiment the antibody is engineered to have an increased proportion of non-fucosylated oligosaccharides in the Fc region, as compared to a non-engineered antibody, by producing the antibody in a host cell having decreased α(1,6)-fucosyltransferase activity.

In one embodiment the disease is a disorder treatable by stimulation of effector cell function. In one embodiment the disease is a cell proliferation disorder. In a particular embodiment the disease is cancer. In a specific embodiment the cancer is selected from the group of lung cancer, colorectal cancer, renal cancer, prostate cancer, breast cancer, head and neck cancer, ovarian cancer, brain cancer, lymphoma, leukemia, and skin cancer. In one embodiment the individual is a mammal. In a particular embodiment the individual is a human.

In another aspect the invention provides a pharmaceutical composition comprising (a) an immunoconjugate comprising at least one antigen-binding moiety and an effector moiety, and (b) an antibody engineered to have increased effector function, in a pharmaceutically acceptable carrier.

The invention also encompasses the use of (a) an immunoconjugate comprising at least one antigen binding moiety and an effector moiety, and (b) an antibody engineered to have increased effector function, for the manufacture of a medicament for the treatment of a disease in an individual.

The invention further provides a method of treating a disease in an individual, comprising administering to the individual a combination of (a) an immunoconjugate comprising at least one antigen binding moiety and an effector moiety, and (b) an antibody engineered to have increased effector function, in a therapeutically effective amount.

Also provided by the invention is a method of stimulating effector cell function in an individual, comprising administering to the individual a combination of (a) an immunoconjugate comprising at least one antigen binding moiety and an effector moiety, and (b) an antibody engineered to have increased effector function, in an amount effective to stimulate effector cell function.

In a further aspect the invention provides a kit intended for the treatment of a disease, comprising in the same or in separate containers (a) an immunoconjugate comprising at least one antigen binding moiety and an effector moiety, (b) an antibody engineered to have increased effector function, and (c) optionally a package insert comprising printed instructions directing the use of the combined treatment as a method for treating the disease.

It is understood that the immunoconjugate and the antibody used in the pharmaceutical composition, use, methods and kit according to the invention may incorporate any of the features, singly or in combination, described in the preceding paragraphs in relation to the antibodies and immunoconjugates useful for the invention.

### SHORT DESCRIPTION OF THE DRAWINGS

FIG. 1. The TNC A2-targeted 2B10 Fab-IL-2-Fab immunoconjugate and the anti-EGFR GlycoMab were tested in the human non-small cell lung cancer (NSCLC) cell line A549, injected i.v. into SCID-human FcγRIII transgenic mice. This tumor model was shown by IHC on fresh frozen tissue to be positive for the A2 domain of Tenascin C. The data shows that the combination of the 2B10 Fab-IL-2-Fab immunoconjugate and the anti-EGFR GlycoMab mediated superior efficacy in terms of enhanced median survival compared to the 2B10 Fab-IL-2-Fab immunoconjugate or the anti-EGFR GlycoMab alone (see Example 1).

FIG. 2. The TNC A2-targeted 2B10 Fab-IL-2-Fab immunoconjugate and the anti-EGFR GlycoMab were tested in the human colorectal LS174T cell line, intrasplenically injected into SCID mice. This tumor model was shown by IHC on fresh frozen tissue to be positive for the A2 domain of Tenascin C. The data shows that the combination of the 2B10 Fab-IL-2-Fab immunoconjugate and the anti-EGFR GlycoMab mediated superior efficacy in terms of enhanced median and overall survival compared to the 2B10 Fab-IL-2-Fab immunoconjugate or the anti-EGFR GlycoMab alone (see Example 2).

FIG. 3. The FAP-targeted 3F2 Fab-IL-2-Fab immunoconjugate and the anti-EGFR GlycoMab were tested in the human renal cell line ACHN, intrarenally injected into SCID mice. This tumor model was shown by IHC on fresh frozen tissue to be positive for FAP. The data shows that the combination of the 3F2 Fab-IL-2-Fab immunoconjugate and the anti-EGFR GlycoMab resulted in synergistically enhanced median and overall survival in SCID mice compared to the 3F2 Fab-IL-2-Fab immunoconjugate or the anti-EGFR GlycoMab alone (see Example 3).

FIG. 4. The FAP-targeted 3F2 Fab-IL-2-Fab immunoconjugate and the anti-EGFR GlycoMab were tested in the human renal cell line ACHN, intrarenally injected into SCID-human FcγRIII transgenic mice. This tumor model was shown by IHC on fresh frozen tissue to be positive for FAP. The data shows that the combination of the 3F2 Fab-IL-2-Fab immunoconjugate and the anti-EGFR GlycoMab mediated superior efficacy in terms of overall survival compared to the 3F2 Fab-IL-2-Fab immunoconjugate or the anti-EGFR GlycoMab alone (see Example 4).

FIG. 5. The TNC A2-targeted 2B10 Fab-IL-2-Fab immunoconjugate and the anti-CD20 GlycoMab were tested in the human mantle cell lymphoma cell line Z138, injected i.v. into SCID-human FcγRIII transgenic mice. This tumor model was shown by IHC on fresh frozen tissue to be positive for TNC A2. The data shows that the combination of the 2B10 Fab-IL-2-Fab immunoconjugate and the anti-CD20-GlycoMab synergistically enhanced median and overall survival compared to the 2B10 Fab-IL-2-Fab immunoconjugate or the anti-CD20-GlycoMab alone (see Example 5).

FIG. 6. The FAP-targeted 28H1 Fab-IL-2-Fab immunoconjugate, comprising the IL-2 quadruple mutant (qm) that lacks binding to CD25, and the anti-EGFR GlycoMab are being tested in the human renal cell line ACHN, intrarenally injected into SCID-human FcγRIII transgenic mice. This tumor model was shown by IHC on fresh frozen tissue to be positive for FAP. The data show that the combination of the 28H1 Fab-IL-2 qm-Fab immunoconjugate and the anti-EGFR GlycoMab mediates superior efficacy in terms of enhanced median survival compared to the 28H1 Fab-IL-2 qm-Fab immunoconjugate or the anti-EGFR GlycoMab alone (see Example 6).

FIG. 7. Increase of K562 tumor cell killing by PBMCs (E:T=10:1, 4 hours) pre-treated for 48 hours with IL-2 (Proleukin), 28H1 Fab-IL2-Fab or 28H1 Fab-IL2 qm-Fab, present in solution (A) or coated to the cell dish (B). Values represent increase in killing in percent, as compared to untreated PBMCs (see Example 8).

FIG. 8. Overall A549 tumor cell killing by PBMCs (E:T=10:1, 4 hours), pre-treated or not for 45 hours with 57 nM FAP-targeted 28H1 Fab-IL2-Fab or 28H1 Fab-IL2 qm-Fab, in the presence of different concentrations of anti-EGFR GlycoMab (see Example 8).

FIG. 9. IFN-γ release by PBMCs during ADCC, after incubation with anti-EGFR GlycoMab (A) or Erbitux (B) alone (5 or 500 ng/ml) or in combination with different concentrations of IL-2 (Proleukin), 28H1 Fab-IL2-Fab or 28H1 Fab-IL2 qm-Fab. A549 cells were used as target cells (E:T=5:1, 21 hours; see Example 8).

FIG. 10. IFN-γ release by PBMCs during antibody-independent killing of A549 tumor cells, after incubation with different concentrations of IL-2 (Proleukin), 28H1 Fab-IL2-Fab or 28H1 Fab-IL2 qm-Fab (E:T=5:1, 21 hours; see Example 8).

### DETAILED DESCRIPTION OF THE INVENTION

In a first aspect the present invention provides a combination of (a) an immunoconjugate comprising at least one antigen-binding moiety and an effector moiety, and (b) an antibody engineered to have increased effector function, for use in treating a disease in an individual in need thereof.

The invention further provides a method of treating a disease in an individual, comprising administering to the individual a combination of (a) an immunoconjugate comprising at least one antigen binding moiety and an effector moiety, and (b) an antibody engineered to have increased effector function, in a therapeutically effective amount.

Also provided by the invention is a method of stimulating effector cell function in an individual, comprising administering to the individual a combination of (a) an immunoconjugate comprising at least one antigen binding moiety and an effector moiety, and (b) an antibody engineered to have increased effector function, in an amount effective to stimulate effector cell function.

### DEFINITIONS

Terms are used herein as generally used in the art, unless otherwise defined in the following.

As used herein, the term “immunoconjugate” refers to a polypeptide molecule that includes at least one effector moiety and at least one antigen binding moiety. In certain embodiments, the immunoconjugate comprises at least one effector moiety, and at least two antigen binding moieties. Particular immunoconjugates according to the invention essentially consist of one effector moiety and two antigen binding moieties joined by one or more linker sequences. The antigen binding moiety can be joined to the effector moiety by a variety of interactions and in a variety of configurations as described herein.

As used herein, the term “antigen binding moiety” refers to a polypeptide molecule that specifically binds to an antigenic determinant. In one embodiment, an antigen binding moiety is able to direct the entity to which it is attached (e.g. an effector moiety or a second antigen binding moiety) to a target site, for example to a specific type of tumor cell or tumor stroma bearing the antigenic determinant. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “control antigen binding moiety” refers to an antigen binding moiety as it would exist free of other antigen binding moieties and effector moieties. For example, when comparing a Fab-IL2-Fab immunoconjugate as described herein with a control antigen binding moiety, the control antigen binding moiety is free Fab, wherein the Fab-IL2-Fab immunoconjugate and the free Fab molecule can both specifically bind to the same antigenic determinant.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen-binding moiety binds, forming an antigen-binding moiety-antigen complex. Useful antigenic determinants can be found, for example, on the surfaces of tumor cells, on the surfaces of virus-infected cells, on the surfaces of other diseased cells, free in blood serum, and/or in the extracellular matrix (ECM).

By “specifically binds” is meant that the binding is selective for the antigen and can be discriminated from unwanted or non-specific interactions. The ability of an antigen-binding moiety to bind to a specific antigenic determinant can be measured either through an enzyme-linked immunosorbent assay (ELISA) or other techniques familiar to one of skill in the art, e.g. surface plasmon resonance technique (analyzed on a BIAcore instrument) (Liljeblad et al., Glyco J 17, 323-329 (2000)), and traditional binding assays (Heeley, Endocr Res 28, 217-229 (2002)).

The terms “anti-[antigen] antibody” and “an antibody that binds to [antigen]” refer to an antibody that is capable of binding the respective antigen with sufficient affinity such that the antibody is useful as a diagnostic and/or therapeutic agent in targeting the antigen. In one embodiment, the extent of binding of an anti-[antigen] antibody to an unrelated protein is less than about 10% of the binding of the antibody to the antigen as measured, e.g., by a radioimmunoassay (RIA). In certain embodiments, an antibody that binds to [antigen] has a dissociation constant (K_{D}) of ≦104, ≦100 nM, ≦10 nM, ≦1 nM, ≦0.1 nM, ≦0.01 nM, or ≦0.001 nM (e.g. 10⁻⁸ M or less, e.g. from 10⁻⁸M to 10⁻¹³ M, e.g., from 10⁻⁹ M to 10⁻¹³ M). It is understood that the above definition is also applicable to antigen-binding moieties that bind to an antigen.

As used herein, the terms “first” and “second” with respect to antigen-binding moieties etc., are used for convenience of distinguishing when there is more than one of each type of moiety. Use of these terms is not intended to confer a specific order or orientation of the immunoconjugate unless explicitly so stated.

As used herein, the term “effector moiety” refers to a polypeptide, e.g., a protein or glycoprotein, that influences cellular activity, for example, through signal transduction or other cellular pathways. Accordingly, the effector moiety of the invention can be associated with receptor-mediated signaling that transmits a signal from outside the cell membrane to modulate a response in a cell bearing one or more receptors for the effector moiety. In one embodiment, an effector moiety can elicit a cytotoxic response in cells bearing one or more receptors for the effector moiety. In another embodiment, an effector moiety can elicit a proliferative response in cells bearing one or more receptors for the effector moiety. In another embodiment, an effector moiety can elicit differentiation in cells bearing receptors for the effector moiety. In another embodiment, an effector moiety can alter expression (i.e. upregulate or downregulate) of an endogenous cellular protein in cells bearing receptors for the effector moiety. Non-limiting examples of effector moieties include cytokines, growth factors, hormones, enzymes, substrates, and cofactors. The effector moiety can be associated with an antigen-binding moiety in a variety of configurations to form an immunoconjugate.

As used herein, the term “cytokine” refers to a molecule that mediates and/or regulates a biological or cellular function or process (e.g. immunity, inflammation, and hematopoiesis). The term “cytokine” as used herein includes “lymphokines,” “chemokines,” “monokines,” and “interleukins”. Examples of useful cytokines include, but are not limited to, GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, and TNF-β. Particular cytokines are IL-2 and IL-12. The term “cytokine” as used herein is meant to also include cytokine analoga comprising one or more amino acid mutations in the amino acid sequences of the corresponding wild-type cytokine, such as for example the IL-2 analoga described in Sauvé et al., Proc Natl Acad Sci USA 88, 4636-40 (1991); Hu et al., Blood 101, 4853-4861 (2003) and US Pat. Publ. No. 2003/0124678; Shanafelt et al., Nature Biotechnol 18, 1197-1202 (2000); Heaton et al., Cancer Res 53, 2597-602 (1993) and U.S. Pat. No. 5,229,109; US Pat. Publ. No. 2007/0036752; WO 2008/0034473; WO 2009/061853; or hereinabove and -below.

As used herein, the term “single-chain” refers to a molecule comprising amino acid monomers linearly linked by peptide bonds. In one embodiment, the effector moiety is a single-chain effector moiety. Non-limiting examples of single-chain effector moieties include cytokines, growth factors, hormones, enzymes, substrates, and cofactors. When the effector moiety is a cytokine and the cytokine of interest is normally found as a multimer in nature, each subunit of the multimeric cytokine is sequentially encoded by the single-chain of the effector moiety. Accordingly, non-limiting examples of useful single-chain effector moieties include GM-CSF, IL-1α, IL-10, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, and TNF-β.

As used herein, the term “control effector moiety” refers to an unconjugated effector moiety. For example, when comparing an IL-2 immunoconjugate as described herein with a control effector moiety, the control effector moiety is free, unconjugated IL-2. Likewise, e.g., when comparing an IL-12 immunoconjugate with a control effector moiety, the control effector moiety is free, unconjugated IL-12 (e.g. existing as a heterodimeric protein wherein the p40 and p35 subunits share only disulfide bond(s)).

As used herein, the term “effector moiety receptor” refers to a polypeptide molecule capable of binding specifically to an effector moiety. For example, where IL-2 is the effector moiety, the effector moiety receptor that binds to an IL-2 molecule (e.g. an immunoconjugate comprising IL-2) is the IL-2 receptor. Similarly, e.g., where IL-12 is the effector moiety of an immunoconjugate, the effector moiety receptor is the IL-12 receptor. Where an effector moiety specifically binds to more than one receptor, all receptors that specifically bind to the effector moiety are “effector moiety receptors” for that effector moiety.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity and comprise an Fc region or a region equivalent to the Fc region of an immunoglobulin

The terms “full-length antibody,” “intact antibody,” and “whole antibody” are used herein interchangeably to refer to an antibody having a structure substantially similar to a native antibody structure or having heavy chains that contain an Fc region as defined herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), single-domain antibodies, and multispecific antibodies formed from antibody fragments. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W. H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular′s “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

The polypeptide sequences of the sequence listing (i.e., SEQ ID NOs 3, 4, 5, 6, 7, 8, 9, etc.) are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cell-mediated cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie et al., Science 247, 1306-10 (1990)).

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the term “effector cells” refers to a population of lymphocytes that display effector moiety receptors, e.g. cytokine receptors, and/or Fc receptors on their surface through which they bind an effector moiety, e.g. a cytokine, and/or an Fc region of an antibody and contribute to the destruction of target cells, e.g. tumor cells. Effector cells may for example mediate cytotoxic or phagocytic effects. Effector cells include, but are not limited to, effector T cells such as CD8⁺ cytotoxic T cells, CD4⁺ helper T cells, γδ T cells, NK cells, lymphokine-activated killer (LAK) cells and macrophages/monocytes. Depending on their receptor expression pattern there may be different subsets of effector cells, i.e. (a) cells that express receptors for a particular effector moiety but no Fc receptors and are stimulated by the immunoconjugates but not the antibodies of the invention (e.g. T cells, expressing IL-2 receptors); (b) cells that express Fc receptors but no receptors for a particular effector moiety and are stimulated by the antibodies but not the immunoconjugates of the invention; and (c) cells that express both Fc receptors and receptors for a particular effector moiety and are simultaneously stimulated by the antibodies and the immunoconjugates of the invention (e.g. NK cells, expressing FcγIII receptors and IL-2 receptors).

As used herein, the terms “engineer, engineered, engineering,” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. “Engineering”, particularly with the prefix “glyco-”, as well as the term “glycosylation engineering” includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity. Glycosylation engineering can be used to obtain a “host cell having increased GnTIII activity” (e.g. a host cell that has been manipulated to express increased levels of one or more polypeptides having β(1,4)-N-acetylglucosaminyltransferase III (GnTIII) activity), a “host cell having increased ManII activity” (e.g. a host cell that has been manipulated to express increased levels of one or more polypeptides having α-mannosidase II (ManII) activity), or a “host cell having decreased α(1,6) fucosyltransferase activity” (e.g. a host cell that has been manipulated to express decreased levels of α(1,6) fucosyltransferase).

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antibodies and immunoconjugates used for the present invention. In one embodiment, the host cell is engineered to allow the production of an antibody with modified oligosaccharides. In certain embodiments, the host cells have been manipulated to express increased levels of one or more polypeptides having β(1,4)-N-acetylglucosaminyltransferase III (GnTIII) activity. In certain embodiments the host cells have been further manipulated to express increased levels of one or more polypeptides having α-mannosidase II (ManII) activity. Host cells include cultured cells, e.g. mammalian cultured cells, such as CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

As used herein, the term “polypeptide having GnTIII activity” refers to polypeptides that are able to catalyze the addition of a N-acetylglucosamine (GlcNAc) residue in β-1,4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependency in a given activity as compared to the GnTIII (i.e. the candidate polypeptide will exhibit greater activity or not more than about 25-fold less and, preferably, not more than about ten-fold less activity, and most preferably, not more than about three-fold less activity relative to the GnTIII). In certain embodiments the polypeptide having GnTIII activity is a fusion polypeptide comprising the catalytic domain of GnTIII and the Golgi localization domain of a heterologous Golgi resident polypeptide. Particularly, the Golgi localization domain is the localization domain of mannosidase II or GnTI, most particularly the localization domain of mannosidase II. Alternatively, the Golgi localization domain is selected from the group consisting of: the localization domain of mannosidase I, the localization domain of GnTII, and the localization domain of α1,6 core fucosyltransferase. Methods for generating such fusion polypeptides and using them to produce antibodies with increased effector functions are disclosed in WO2004/065540, U.S. Provisional Pat. Appl. No. 60/495,142 and U.S. Pat. Appl. Publ. No. 2004/0241817, the entire contents of which are expressly incorporated herein by reference.

As used herein, the term “Golgi localization domain” refers to the amino acid sequence of a Golgi resident polypeptide which is responsible for anchoring the polypeptide to a location within the Golgi complex. Generally, localization domains comprise amino terminal “tails” of an enzyme.

As used herein, the term “polypeptide having ManII activity” refers to polypeptides that are able to catalyze the hydrolysis of the terminal 1,3- and 1,6-linked α-D-mannose residues in the branched GlcNAcMan₅GlcNAc₂ mannose intermediate of N-linked oligosaccharides. This includes polypeptides exhibiting enzymatic activity similar to, but not necessarily identical to, an activity of Golgi α-mannosidase II, also known as mannosyl oligosaccharide 1,3-1,6-α-mannosidase II (EC 3.2.1.114), according to the Nomenclature Committee of the International Union of Biochemistry and Molecular Biology (NC-IUBMB).

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. As used herein, the term “increased ADCC” is defined as either an increase in the number of target cells that are lysed in a given time, at a given concentration of antibody in the medium surrounding the target cells, by the mechanism of ADCC defined above, and/or a reduction in the concentration of antibody, in the medium surrounding the target cells, required to achieve the lysis of a given number of target cells in a given time, by the mechanism of ADCC. The increase in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g. to express the glycosyltransferase, GnTIII, or other glycosyltransferases) by the methods described herein, is relative to the ADCC mediated by the same antibody produced by the same type of non-engineered host cells.

By “antibody having increased antibody dependent cell-mediated cytotoxicity (ADCC)” is meant an antibody having increased ADCC as determined by any suitable method known to those of ordinary skill in the art. One accepted in vitro ADCC assay is as follows:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
i) the PBMCs are isolated using standard density centrifugation procedures and are suspended at 5×10⁶ cells/ml in RPMI cell culture medium;
ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
iii) 100 microliters of the final target cell suspension above are transferred to each well of a 96-well microtiter plate;
iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
v) for the maximum release (MR) controls, 3 additional wells in the plate containing the labeled target cells, receive 50 microliters of a 2% (V/V) aqueous solution of non-ionic detergent (Nonidet, Sigma, St. Louis), instead of the antibody solution (point iv above);
vi) for the spontaneous release (SR) controls, 3 additional wells in the plate containing the labeled target cells, receive 50 microliters of RPMI cell culture medium instead of the antibody solution (point iv above);
vii) the 96-well microtiter plate is then centrifuged at 50×g for 1 minute and incubated for 1 hour at 4° C.;
viii) 50 microliters of the PBMC suspension (point i above) are added to each well to yield an effector:target cell ratio of 25:1 and the plates are placed in an incubator under 5% CO₂ atmosphere at 37° C. for 4 hours;
ix) the cell-free supernatant from each well is harvested and the experimentally released radioactivity (ER) is quantified using a gamma counter;
x) the percentage of specific lysis is calculated for each antibody concentration according to the formula (ER−MR)/(MR−SR)×100, where ER is the average radioactivity quantified (see point ix above) for that antibody concentration, MR is the average radioactivity quantified (see point ix above) for the MR controls (see point v above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. The increase in ADCC is relative to the ADCC, measured with the above assay, mediated by the same antibody, produced by the same type of host cells, using the same standard production, purification, formulation and storage methods, which are known to those skilled in the art, but that has not been engineered.

As used herein, “combination” (and grammatical variations thereof such as “combine” or “combining”) encompasses combinations of an immunoconjugate and an antibody according to the invention wherein the immunoconjugate and the antibody are in the same or in different containers, in the same or in different pharmaceutical formulations, administered together or separately, administered simultaneously or sequentially, in any order, and administered by the same or by different routes, provided that the immunoconjugate and the antibody can simultaneously exert their biological effects in the body, i.e. simultaneously stimulate effector cells. For example “combining” an immunoconjugate and an antibody according to the invention may mean first administering the immunoconjugate in a particular pharmaceutical formulation, followed by administration of the antibody in another pharmaceutical formulation, or vice versa.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease. A therapeutically effective amount of a combination of several active ingredients may be a therapeutically effective amount of each of the active ingredients. Alternatively, to reduce the side effects caused by the treatment, a therapeutically effective amount of a combination of several active ingredients may be amounts of the individual active ingredients that are effective to produce an additive, or a superadditive or synergistic effect, and that in combination are therapeutically effective, but which may be sub-therapeutic amounts of one or several of the active ingredients if they were used alone.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, combinations of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

Immunoconjugates

Immunoconjugates useful in the present invention are polypeptide molecules that comprise at least one effector moiety and at least one antigen-binding moiety.

Immunoconjugates can be prepared either by chemically conjugating the effector moiety to the antigen-binding moiety, or by expressing the effector moiety and the antigen-binding moiety as a fusion protein (see, e.g. Nakamura and Kubo, Cancer 80, 2650-2655 (1997); and Becker et al., Proc Natl Acad Sci USA 93, 7826-7831 (1996)). For use in the present invention, immunoconjugates expressed as fusion proteins are generally preferred. Accordingly, in certain embodiments the effector moiety shares an amino- or carboxy-terminal peptide bond with the antigen-binding moiety (i.e. the immunoconjugate is a fusion protein). In such immunoconjugates, an effector moiety may for example be fused to an immunoglobulin heavy or light chain. Particularly useful in the present invention are immunoconjugates comprising an antibody fragment, such as a Fab or a scFv molecule, as antigen binding moiety. Exemplary antibody fragment/cytokine immunoconjugates are described e.g. in Savage et al., Br J Cancer 67, 304-310 (1993); Yang et al., Mol. Immunol. 32, 873-881 (1995); PCT publication WO 2001/062298 A2; U.S. Pat. No. 5,650,150; PCT publication WO 2006/119897 A2; and PCT publication WO 99/29732 A2.

In one embodiment, the effector moiety is a single-chain effector moiety. In one embodiment the effector moiety is a cytokine. In one embodiment, the immunoconjugate comprises at least two antigen-binding moieties. The antigen-binding moieties and effector moieties of the immunoconjugate include those that are described in detail herein above and below. The antigen-binding moiety of the immunoconjugate can be directed against a variety of target molecules (e.g. an antigenic determinant on a protein molecule expressed on a tumor cell or tumor stroma). Non-limiting examples of antigen binding moieties are described herein. Particularly useful immunoconjugates as described herein typically exhibit one or more of the following properties: high specificity of action, reduced toxicity and/or improved stability, particularly as compared to immunoconjugates of different configurations targeting the same antigenic determinants and carrying the same effector moieties. Particular immunoconjugates for use in the present invention are further described in PCT publication number WO 2011/020783, the entire contents of which are incorporated herein by reference.

Immunoconjugate Formats

The immunoconjugates described in PCT publication number WO 2011/020783 comprise at least two antigen binding domains. Thus, in one embodiment, the immunoconjugate comprises at least a first effector moiety and at least a first and a second antigen binding moiety. In one embodiment, the first effector moiety is a single chain effector moiety. In one embodiment, the first and second antigen binding moiety are independently selected from the group consisting of a scFv molecule and a Fab molecule. In a particular embodiment each of the first and the second antigen-binding moieties is a Fab molecule. In another embodiment each of the first and the second antigen-binding moieties is a scFv molecule. In a specific embodiment, the first effector moiety shares an amino- or carboxy-terminal peptide bond with the first antigen binding moiety, and the second antigen binding moiety shares an amino- or carboxy-terminal peptide bond with either i) the first effector moiety or ii) the first antigen binding moiety. In a particular embodiment, the immunoconjugate consists essentially of a first single-chain effector moiety and first and second antigen binding moieties. In an even more particular embodiment each of the first and second antigen-binding moieties is a Fab molecule.

In one embodiment, a first effector moiety shares a carboxy-terminal peptide bond with a first antigen binding moiety and further shares an amino-terminal peptide bond with a second antigen binding moiety. In another embodiment, a first antigen binding moiety shares a carboxy-terminal peptide bond with a first effector moiety, particularly a single chain effector moiety, and further shares an amino-terminal peptide bond with a second antigen binding moiety. In another embodiment, a first antigen binding moiety shares an amino-terminal peptide bond with a first effector moiety, particularly a single chain effector moiety, and further shares a carboxy-terminal peptide with a second antigen binding moiety.

In one embodiment, an effector moiety, particularly a single chain effector moiety, shares a carboxy-terminal peptide bond with a first heavy chain variable region and further shares an amino-terminal peptide bond with a second heavy chain variable region. In another embodiment, an effector moiety, particularly a single chain effector moiety, shares a carboxy-terminal peptide bond with a first light chain variable region and further shares an amino-terminal peptide with a second light chain variable region. In another embodiment, a first heavy or light chain variable region is joined by a carboxy-terminal peptide bond to a first effector moiety, particularly a single chain effector moiety, and is further joined by an amino-terminal peptide bond to a second heavy or light chain variable region. In another embodiment, a first heavy or light chain variable region is joined by an amino-terminal peptide bond to a first effector moiety, particularly a single chain effector moiety, and is further joined by a carboxy-terminal peptide bond to a second heavy or light chain variable region.

In a particular embodiment, an effector moiety, particularly a single chain effector moiety, shares a carboxy-terminal peptide bond with a first Fab heavy or light chain and further shares an amino-terminal peptide bond with a second Fab heavy or light chain. In another embodiment, a first Fab heavy or light chain shares a carboxy-terminal peptide bond with a first single-chain effector moiety and further shares an amino-terminal peptide bond with a second Fab heavy or light chain. In other embodiments, a first Fab heavy or light chain shares an amino-terminal peptide bond with a first single-chain effector moiety and further shares a carboxy-terminal peptide bond with a second Fab heavy or light chain.

In one embodiment, the immunoconjugate comprises at least a first effector moiety sharing an amino-terminal peptide bond with one or more scFv molecules and wherein the first effector moiety further shares a carboxy-terminal peptide bond with one or more scFv molecules. In a particular embodiment, the effector moiety is a single chain effector moiety.

In another embodiment, the immunoconjugate comprises at least a first effector moiety, particularly a single chain effector moiety, and first and second antigen binding moieties, wherein each of the antigen binding moieties includes an scFv molecule joined at its carboxy-terminal amino acid to a constant region that includes an immunoglobulin constant domain, and wherein the first antigen binding moiety is joined at its constant region carboxy-terminal amino acid to the amino-terminal amino acid of the first effector moiety, and wherein the first and second antigen binding moieties are covalently linked through at least one disulfide bond. In a particular embodiment, the constant region is independently selected from the group consisting of IgG CH1, IgG CH2, IgG CH3, IgG Cₖₐₚₚₐ, IgG C_{lambda} and IgE CH4 domains. In one embodiment, the immunoglobulin domain of the first antigen binding moiety is covalently linked to the immunoglobulin domain of the second antigen binding moiety through a disulfide bond. In one embodiment, at least one disulfide bond is located carboxy-terminal of the immunoglobulin domains of the first and second antigen binding moieties. In another embodiment, at least one disulfide bond is located amino-terminal of the immunoglobulin domains of the first and second antigen binding moieties. In another embodiment, at least two disulfide bonds are located amino-terminal of the immunoglobulin domains of the first and second antigen binding moieties.

In a specific embodiment, the immunoconjugate comprises first and second antigen binding moieties, each comprising an scFv molecule joined at its carboxy-terminal amino acid to a constant region that comprises an IgG CH1 domain, wherein the first antigen binding moiety is joined at its constant region carboxy-terminal amino acid to the amino-terminal amino acid of the first effector moiety, particularly a single chain effector moiety, and wherein the first and second antigen binding moieties are covalently linked through at least one disulfide bond. The second antigen binding moiety of the immunoconjugate can be further joined at its carboxy-terminal amino acid to the amino-terminal amino acid of a second effector moiety. In one embodiment, the second effector moiety is a single chain effector moiety.

In a specific embodiment, the immunoconjugate comprises first and second antigen binding moieties each comprising an scFv molecule joined at its carboxy-terminal amino acid to a constant region that comprises an IgG Cₖₐₚₚₐ domain, wherein the first antigen binding moiety is joined at its constant region carboxy-terminal amino acid to the amino-terminal amino acid of the first effector moiety, particularly a single chain effector moiety, and wherein the first and second antigen binding moieties are covalently linked through at least one disulfide bond. The second antigen binding moiety of the immunoconjugate can be further joined at its carboxy-terminal amino acid to the amino-terminal amino acid of a second effector moiety. In one embodiment, the second effector moiety is a single chain effector moiety.

In another specific embodiment, the immunoconjugate comprises first and second antigen binding moieties, each comprising an scFv molecule joined at its carboxy-terminal amino acid to a constant region that comprises an IgE CH4 domain, wherein the first antigen binding moiety is joined at its constant region carboxy-terminal amino acid to the amino-terminal amino acid of the first effector moiety, particularly a single chain effector moiety, and wherein the first and second antigen binding moieties are covalently linked through at least one disulfide bond. The second antigen binding moiety of the immunoconjugate can be further joined at its carboxy-terminal amino acid to the amino-terminal amino acid of a second effector moiety. In one embodiment, the second effector moiety is a single chain effector moiety.

In another specific embodiment, the immunoconjugate comprises first and second antigen binding moieties each, comprising an scFv molecule joined at its carboxy-terminal amino acid to an IgE CH3 domain, wherein the first antigen binding moiety is joined at its carboxy-terminal amino acid to the amino-terminal amino acid of the first effector moiety, particularly a single chain effector moiety, and wherein the first and second antigen binding moieties are covalently linked through at least one disulfide bond. The second antigen binding moiety of the immunoconjugate can be further joined at its carboxy-terminal amino acid to the amino-terminal amino acid of a second effector moiety. In one embodiment, the second effector moiety is a single chain effector moiety.

In another embodiment, the immunoconjugate comprises first and second effector moieties, and first and second antigen binding moieties, wherein each of the antigen binding moieties comprises an Fab molecule joined at its heavy or light chain carboxy-terminal amino acid to an IgG1 CH3 domain, and wherein each of the IgG1 CH3 domains is joined at its respective carboxy-terminal amino acid to the amino-terminal amino acid of one of the effector moieties, and wherein the first and second antigen binding moieties are covalently linked through at least one disulfide bond. In a particular embodiment, the first and/or second effector moiety is a single chain effector moiety. In a further embodiment, the IgG1 CH3 domains of the antigen binding moieties may be joined by disulfide bond. In another embodiment, at least one disulfide bond is located carboxy-terminal of the IgG1 CH3 domains of the first and second antigen binding moieties. In another embodiment, at least one disulfide bond is located amino-terminal of the IgG1 CH3 domains of the first and second antigen binding moieties. In another embodiment, at least two disulfide bonds are located amino-terminal of the IgG1 CH3 domains of the first and second antigen binding moieties.

In some embodiments, the immunoconjugate comprises one or more proteolytic cleavage sites located between effector moieties and antigen binding moieties. Components of the immunoconjugate (e.g., antigen binding moieties and/or effector moieties) may be linked directly or through various linkers, particularly peptide linkers comprising one or more amino acids, typically about 2-20 amino acids, that are described herein or are known in the art. Suitable, non-immunogenic linker peptides include, for example, (G4S)ₙ, (SG₄)ₙ or G₄(SG₄)ₙ linker peptides, wherein n is generally a number between 1 and 10, typically between 2 and 4.

Antigen Binding Moieties

The antigen-binding moiety of the immunoconjugate of the invention is generally a polypeptide molecule that binds to a specific antigenic determinant and is able to direct the entity to which it is attached (e.g. an effector moiety or a second antigen binding moiety) to a target site, for example to a specific type of tumor cell or tumor stroma that bears the antigenic determinant. The immunoconjugate can bind to antigenic determinants found, for example, on the surfaces of tumor cells, on the surfaces of virus-infected cells, on the surfaces of other diseased cells, free in blood serum, and/or in the extracellular matrix (ECM). Non-limiting examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, PiA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2. Non-limiting examples of viral antigens include influenza virus hemagglutinin, Epstein-Barr virus LMP-1, hepatitis C virus E2 glycoprotein, HIV gp160, and HIV gp120. Non-limiting examples of ECM antigens include syndecan, heparanase, integrins, osteopontin, link, cadherins, laminin, laminin type EGF, lectin, fibronectin, notch, tenascin, and matrixin. The immunoconjugates of the invention can bind to the following specific non-limiting examples of cell surface antigens: FAP, Her2, EGFR, IGF-1R, CD2 (T-cell surface antigen), CD3 (heteromultimer associated with the TCR), CD22 (B-cell receptor), CD23 (low affinity IgE receptor), CD25 (IL-2 receptor a chain), CD30 (cytokine receptor), CD33 (myeloid cell surface antigen), CD40 (tumor necrosis factor receptor), IL-6R (IL6 receptor), CD20, MCSP, c-Met, CUB domain-containing protein-1 (CDCP1), and PDGFβR (β platelet-derived growth factor receptor).

In certain embodiments the antigen-binding moiety is directed to an antigen presented on a tumor cell or in a tumor cell environment. In a specific embodiment the antigen-binding moiety is directed to an antigen selected from the group of Fibroblast Activation Protein (FAP), the A1 domain of Tenascin-C (TNC A1), the A2 domain of Tenascin-C (TNC A2), the Extra Domain B of Fibronectin (EDB), Carcinoembryonic Antigen (CEA) and Melanoma-associated Chondroitin Sulfate Proteoglycan (MCSP).

In one embodiment, the immunoconjugate of the invention comprises two or more antigen binding moieties, wherein each of these antigen binding moieties specifically binds to the same antigenic determinant. In another embodiment, the immunoconjugate of the invention comprises two or more antigen binding moieties, wherein each of these antigen binding moieties specifically binds to different antigenic determinants.

The antigen binding moiety can be any type of antibody or fragment thereof that retains specific binding to an antigenic determinant. In one embodiment the antigen-binding moiety is an antibody or an antibody fragment. Antibody fragments include, but are not limited to, V_{H} fragments, V_{L} fragments, Fab fragments, F(ab′)₂ fragments, scFv fragments, Fv fragments, minibodies, diabodies, triabodies, and tetrabodies (see e.g. Hudson and Souriau, Nature Med 9, 129-134 (2003)). Particularly useful antibody fragments are Fab fragments and scFv fragments. Accordingly, in one embodiment the antigen-binding moiety is selected from a Fab molecule and a scFv molecule. In one embodiment the antigen-binding moiety is a Fab molecule. In another embodiment the antigen-binding moiety is a scFv molecule.

In one embodiment, the immunoconjugate comprises at least one, typically two or more antigen binding moieties that are specific for the Extra Domain B of fibronectin (EDB). In another embodiment, the immunoconjugate comprises at least one, typically two or more antigen binding moieties that can compete with monoclonal antibody L19 for binding to an epitope of EDB. See, e.g., PCT publication WO 2007/128563 A1 (incorporated herein by reference in its entirety). In yet another embodiment, the immunoconjugate comprises a polypeptide sequence wherein a first Fab heavy chain derived from the L19 monoclonal antibody shares a carboxy-terminal peptide bond with an IL-2 molecule which in turn shares a carboxy-terminal peptide bond with a second Fab heavy chain derived from the L19 monoclonal antibody. In yet another embodiment, the immunoconjugate comprises a polypeptide sequence wherein a first Fab heavy chain derived from the L19 monoclonal antibody shares a carboxy-terminal peptide bond with an IL-12 molecule which in turn shares a carboxy-terminal peptide bond with a second Fab heavy chain derived from the L19 monoclonal antibody. In yet another embodiment, the immunoconjugate comprises a polypeptide sequence wherein a first Fab heavy chain derived from the L19 monoclonal antibody shares a carboxy-terminal peptide bond with an IFN a molecule which in turn shares a carboxy-terminal peptide bond with a second Fab heavy chain derived from the L19 monoclonal antibody. In yet another embodiment, the immunoconjugate comprises a polypeptide sequence wherein a first Fab heavy chain derived from the L19 monoclonal antibody shares a carboxy-terminal peptide bond with a GM-CSF molecule which in turn shares a carboxy-terminal peptide bond with a second Fab heavy chain derived from the L19 monoclonal antibody. In a further embodiment, the immunoconjugate comprises a polypeptide sequence wherein a first scFv derived from the L19 monoclonal antibody shares a carboxy-terminal peptide bond with an IL-2 molecule which in turn shares a carboxy-terminal peptide bond with a second scFv derived from the L19 monoclonal antibody. In a more specific embodiment, the immunoconjugate comprises the polypeptide sequence of SEQ ID NO: 91 or a variant thereof that retains functionality. In another embodiment, the immunoconjugate comprises a Fab light chain derived from the L19 monoclonal antibody. In a more specific embodiment, the immunoconjugate comprises a polypeptide sequence that is at least about 80%, 85%, 90%, 95%, 96%, 97%, 98%, 99% or 100% identical to SEQ ID NO: 92 or a variant thereof that retains functionality. In yet another embodiment, the immunoconjugate comprises two polypeptide sequences that are at least about 80%, 85%, 90%, 95%, 96%, 97%, 98%, 99% or 100% identical to SEQ ID NO: 91 and SEQ ID NO: 92 or variants thereof that retain functionality. In a more specific embodiment, the immunoconjugate comprises a polypeptide sequence that is at least about 80%, 85%, 90%, 95%, 96%, 97%, 98%, 99% or 100% identical to SEQ ID NO: 98 or a variant thereof that retains functionality. In yet another embodiment, the immunoconjugate comprises two polypeptide sequences that are at least about 80%, 85%, 90%, 95%, 96%, 97%, 98%, 99% or 100% identical to SEQ ID NO: 98 and SEQ ID NO: 92 or variants thereof that retain functionality. In a more specific embodiment, the immunoconjugate comprises a polypeptide sequence that is at least about 80%, 85%, 90%, 95%, 96%, 97%, 98%, 99% or 100% identical to SEQ ID NO: 99 or a variant thereof that retains functionality. In yet another embodiment, the immunoconjugate comprises two polypeptide sequences that are at least about 80%, 85%, 90%, 95%, 96%, 97%, 98%, 99% or 100% identical to SEQ ID NO: 99 and SEQ ID NO: 92 or variants thereof that retain functionality. In a more specific embodiment, the immunoconjugate comprises a polypeptide sequence that is at least about 80%, 85%, 90%, 95%, 96%, 97%, 98%, 99% or 100% identical to SEQ ID NO: 100 or a variant thereof that retains functionality. In yet another embodiment, the immunoconjugate comprises two polypeptide sequences that are at least about 80%, 85%, 90%, 95%, 96%, 97%, 98%, 99% or 100% identical to SEQ ID NO: 100 and SEQ ID NO: 92 or variants thereof that retain functionality. In a more specific embodiment, the immunoconjugate comprises a polypeptide sequence that is at least about 80%, 85%, 90%, 95%, 96%, 97%, 98%, 99% or 100% identical to SEQ ID NO: 101 or a variant thereof that retains functionality. In yet another embodiment, the immunoconjugate comprises two polypeptide sequences that are at least about 80%, 85%, 90%, 95%, 96%, 97%, 98%, 99% or 100% identical to SEQ ID NO: 101 and SEQ ID NO: 92 or variants thereof that retain functionality. In another specific embodiment, the polypeptides are covalently linked, e.g., by a disulfide bond.

In one embodiment, the immunoconjugate of the invention comprises at least one, typically two or more antigen binding moieties that are specific for the A1 domain of Tenascin (TNC-A1). In another embodiment, the immunoconjugate comprises at least one, typically two or more antigen binding moieties that can compete with monoclonal antibody F16 for binding to an epitope of TNC-A1. See, e.g., PCT Publication WO 2007/128563 A1 (incorporated herein by reference in its entirety). In one embodiment, the immunoconjugate comprises at least one, typically two or more antigen binding moieties that are specific for the A1 and/or the A4 domain of Tenascin (TNC-A1 or TNC-A4 or TNC-A1/A4). In another embodiment, the immunoconjugate comprises a polypeptide sequence wherein a first Fab heavy chain specific for the A1 domain of Tenascin shares a carboxy-terminal peptide bond with an IL-2 molecule, an IL-12 molecule, an IFN a molecule or a GM-CSF molecule, which in turn shares a carboxy-terminal peptide bond with a second Fab heavy chain specific for the A1 domain of Tenascin. In yet another embodiment, the immunoconjugate comprises a polypeptide sequence wherein a first Fab heavy chain specific for the A1 domain of Tenascin shares a carboxy-terminal peptide bond with an IL-2 molecule which in turn shares a carboxy-terminal peptide bond with a second Fab heavy chain specific for the A1 domain of Tenascin. In a further embodiment, the immunoconjugate comprises a polypeptide sequence wherein a first scFv specific for the A1 domain of Tenascin shares a carboxy-terminal peptide bond with an IL-2 molecule which in turn shares a carboxy-terminal peptide bond with a second scFv specific for the A1 domain of Tenascin. In a specific embodiment, the antigen binding moieties of the immunoconjugate comprise a heavy chain variable region sequence that is at least about 80%, 85%, 90%, 95%, 96%, 97%, 98%, 99% or 100% identical to either SEQ ID NO: 8 or SEQ ID NO: 9, or variants thereof that retain functionality. In another specific embodiment, the antigen binding moieties of the immunoconjugate comprise a light chain variable region sequence that is at least about 80%, 85%, 90%, 95%, 96%, 97%, 98%, 99% or 100% identical to either SEQ ID NO: 6 or SEQ ID NO: 7, or variants thereof that retain functionality. In a more specific embodiment, the antigen binding moieties of the immunoconjugate comprise a heavy chain variable region sequence that is at least about 80%, 85%, 90%, 95%, 96%, 97%, 98%, 99% or 100% identical to either SEQ ID NO: 8 or SEQ ID NO: 9 or variants thereof that retain functionality, and a light chain variable region sequence that is at least about 80%, 85%, 90%, 95%, 96%, 97%, 98%, 99% or 100% identical to either SEQ ID NO: 6 or SEQ ID NO: 7 or variants thereof that retain functionality. In another specific embodiment, the immunoconjugate comprises a polypeptide sequence that is at least about 80%, 85%, 90%, 95%, 96%, 97%, 98%, 99% or 100% identical to SEQ ID NO: 95 or variants thereof that retain functionality. In another specific embodiment, the immunoconjugate of the invention comprises a polypeptide sequence that is at least about 80%, 85%, 90%, 95%, 96%, 97%, 98%, 99% or 100% identical to either SEQ ID NO: 96 or SEQ ID NO: 105, or variants thereof that retain functionality. In yet another specific embodiment, the immunoconjugate of the invention comprises a polypeptide sequence that is at least about 80%, 85%, 90%, 95%, 96%, 97%, 98%, 99% or 100% identical to either SEQ ID NO: 97 or SEQ ID NO: 115 or variants thereof that retain functionality. In a more specific embodiment, the immunoconjugate of the present invention comprises two polypeptide sequences that are at least about 80%, 85%, 90%, 95%, 96%, 97%, 98%, 99% or 100% identical to SEQ ID NO: 96 and SEQ ID NO: 97 or variants thereof that retain functionality. In another specific embodiment, the immunoconjugate of the present invention comprises two polypeptide sequences that are at least about 80%, 85%, 90%, 95%, 96%, 97%, 98%, 99% or 100% identical to SEQ ID NO: 105 and SEQ ID NO: 115 or variants thereof that retain functionality.

In one embodiment, the immunoconjugate comprises at least one, typically two or more antigen binding moieties that are specific for the A2 domain of Tenascin (TNC-A2). In another embodiment, the immunoconjugate comprises a polypeptide sequence wherein a first Fab heavy chain specific for the A2 domain of Tenascin shares a carboxy-terminal peptide bond with an IL-2 molecule, an IL-12 molecule, an IFN a molecule or a GM-CSF molecule, which in turn shares a carboxy-terminal peptide bond with a second Fab heavy chain specific for the A2 domain of Tenascin. In yet another embodiment, the immunoconjugate comprises a polypeptide sequence wherein a first Fab heavy chain specific for the A2 domain of Tenascin shares a carboxy-terminal peptide bond with an IL-2 molecule, which in turn shares a carboxy-terminal peptide bond with a second Fab heavy chain specific for the A2 domain of Tenascin. In a specific embodiment, the antigen binding moieties of the immunoconjugate comprise a heavy chain variable region sequence that is at least about 80%, 85%, 90%, 95%, 96%, 97%, 98%, 99% or 100% identical to a sequence selected from the group of SEQ ID NO: 5, SEQ ID NO: 71, SEQ ID NO: 73, SEQ ID NO: 75, SEQ ID NO: 77, SEQ ID NO: 79, SEQ ID NO: 81, SEQ ID NO: 83 and SEQ ID NO: 85, or variants thereof that retain functionality. In another specific embodiment, the antigen binding moieties of the immunoconjugate comprise a light chain variable region sequence that is at least about 80%, 85%, 90%, 95%, 96%, 97%, 98%, 99% or 100% identical to a sequence selected from the group of SEQ ID NO: 3, SEQ ID NO: 4; SEQ ID NO: 70, SEQ ID NO: 72, SEQ ID NO: 74, SEQ ID NO: 76, SEQ ID NO: 78, SEQ ID NO: 80, SEQ ID NO: 82 and SEQ ID NO: 84, or variants thereof that retain functionality. In a more specific embodiment, the antigen binding moieties of the immunoconjugate comprise a heavy chain variable region sequence that is at least about 80%, 85%, 90%, 95%, 96%, 97%, 98%, 99% or 100% identical to a sequence selected from the group of SEQ ID NO: 5, SEQ ID NO: 71, SEQ ID NO: 73, SEQ ID NO: 75, SEQ ID NO: 77, SEQ ID NO: 79, SEQ ID NO: 81, SEQ ID NO: 83 and SEQ ID NO: 85, or variants thereof that retain functionality, and a light chain variable region sequence that is at least about 80%, 85%, 90%, 95%, 96%, 97%, 98%, 99% or 100% identical to a sequence selected from the group of SEQ ID NO: 3, SEQ ID NO: 4; SEQ ID NO: 70, SEQ ID NO: 72, SEQ ID NO: 74, SEQ ID NO: 76, SEQ ID NO: 78, SEQ ID NO: 80, SEQ ID NO: 82 and SEQ ID NO: 84, or variants thereof that retain functionality. In another specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 117, SEQ ID NO: 118 and SEQ ID NO: 119, or variants thereof that retain functionality. In another specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 120, SEQ ID NO: 121 and SEQ ID NO: 122, or variants thereof that retain functionality. In a more specific embodiment, the immunoconjugate of the present invention comprises a polypeptide sequence that is at least about 80%, 85%, 90%, 95%, 96%, 97%, 98%, 99% or 100% identical to a sequence selected from the group of SEQ ID NO: 117, SEQ ID NO: 118, and SEQ ID NO: 119 or variants thereof that retain functionality, and a polypeptide sequence that is at least about 80%, 85%, 90%, 95%, 96%, 97%, 98%, 99% or 100% identical to a sequence selected from the group of SEQ ID NO: 120, SEQ ID NO: 121 and SEQ ID NO: 122 or variants thereof that retain functionality. In another specific embodiment, the immunoconjugate of the present invention comprises two polypeptide sequences that are at least about 80%, 85%, 90%, 95%, 96%, 97%, 98%, 99% or 100% identical to SEQ ID NO: 117 and either SEQ ID NO: 121 or SEQ ID NO: 122, or variants thereof that retain functionality. In yet another specific embodiment, the immunoconjugate of the present invention comprises two polypeptide sequences that are at least about 80%, 85%, 90%, 95%, 96%, 97%, 98%, 99% or 100% identical to SEQ ID NO: 118 and either SEQ ID NO: 120 or SEQ ID NO: 121, or variants thereof that retain functionality. In another specific embodiment, the immunoconjugate of the present invention comprises two polypeptide sequences that are at least about 80%, 85%, 90%, 95%, 96%, 97%, 98%, 99% or 100% identical to SEQ ID NO: 119 and SEQ ID NO: 120, or variants thereof that retain functionality.

In one embodiment, the immunoconjugate comprises at least one, typically two or more antigen binding moieties that are specific for the Fibroblast Activated Protein (FAP). In another embodiment, the immunoconjugate comprises a polypeptide sequence wherein a first Fab heavy chain specific for FAP shares a carboxy-terminal peptide bond with an IL-2 molecule, an IL-12 molecule, an IFN a molecule or a GM-CSF molecule, which in turn shares a carboxy-terminal peptide bond with a second Fab heavy chain specific for FAP. In yet another embodiment, the immunoconjugate comprises a polypeptide sequence wherein a first Fab heavy chain specific for FAP shares a carboxy-terminal peptide bond with an IL-2 molecule, which in turn shares a carboxy-terminal peptide bond with a second Fab heavy chain specific for FAP. In another embodiment, the immunoconjugate comprises a polypeptide sequence wherein a first Fab heavy chain specific for FAP shares a carboxy-terminal peptide bond with an IL-12 molecule, which in turn shares a carboxy-terminal peptide bond with a second Fab heavy chain specific for FAP. In a specific embodiment, the antigen binding moieties of the immunoconjugate comprise a heavy chain variable region sequence that is at least about 80%, 85%, 90%, 95%, 96%, 97%, 98%, 99% or 100% identical to a sequence selected from the group consisting of SEQ ID NO: 12, SEQ ID NO: 14, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67 and SEQ ID NO: 69, or variants thereof that retain functionality. In another specific embodiment, the antigen binding moieties of the immunoconjugate comprise a light chain variable region sequence that is at least about 80%, 85%, 90%, 95%, 96%, 97%, 98%, 99% or 100% identical to a sequence selected from the group consisting of: SEQ ID NO: 10, SEQ ID NO: 11, SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66 and SEQ ID NO: 68, or variants thereof that retain functionality. In a more specific embodiment, the antigen binding moieties of the immunoconjugate comprise a heavy chain variable region sequence that is at least about 80%, 85%, 90%, 95%, 96%, 97%, 98%, 99% or 100% identical to a sequence selected from the group consisting of SEQ ID NO: 12, SEQ ID NO: 14, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67 and SEQ ID NO: 69, or variants thereof that retain functionality, and a light chain variable region sequence that is at least about 80%, 85%, 90%, 95%, 96%, 97%, 98%, 99% or 100% identical to a sequence selected from the group consisting of: SEQ ID NO: 10, SEQ ID NO: 11, SEQ ID NO: 13, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66 and SEQ ID NO: 68, or variants thereof that retain functionality. In another specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 102, SEQ ID NO: 103, SEQ ID NO: 104, SEQ ID NO: 106, SEQ ID NO: 107, SEQ ID NO: 108, SEQ ID NO: 109, SEQ ID NO: 110 and SEQ ID NO: 111, or variants thereof that retain functionality. In yet another specific embodiment, the immunoconjugate of the invention comprises a polypeptide sequence that is at least about 80%, 85%, 90%, 95%, 96%, 97%, 98%, 99% or 100% identical to a sequence selected from the group of SEQ ID NO: 112, SEQ ID NO: 113, SEQ ID NO: 114 and SEQ ID NO: 116 or variants thereof that retain functionality. In a more specific embodiment, the immunoconjugate of the present invention comprises a polypeptide sequence that is at least about 80%, 85%, 90%, 95%, 96%, 97%, 98%, 99% or 100% identical to a sequence selected from the group of SEQ ID NO: 103, SEQ ID NO: 107 and SEQ ID NO: 108 or variants thereof that retain functionality, and a polypeptide sequence that is at least about 80%, 85%, 90%, 95%, 96%, 97%, 98%, 99% or 100% identical to SEQ ID NO: 113 or variants thereof that retain functionality. In another specific embodiment, the immunoconjugate of the present invention comprises a polypeptide sequence that is at least about 80%, 85%, 90%, 95%, 96%, 97%, 98%, 99% or 100% identical to a sequence selected from the group of SEQ ID NO: 102, SEQ ID NO: 109, SEQ ID NO: 110 and SEQ ID NO: 111 or variants thereof that retain functionality, and a polypeptide sequence that is at least about 80%, 85%, 90%, 95%, 96%, 97%, 98%, 99% or 100% identical to SEQ ID NO: 112 or variants thereof that retain functionality. In a further specific embodiment, the immunoconjugate of the present invention comprises two polypeptide sequences that are at least about 80%, 85%, 90%, 95%, 96%, 97%, 98%, 99% or 100% identical to SEQ ID NO: 104 and SEQ ID NO: 114 or variants thereof that retain functionality. In yet another specific embodiment, the immunoconjugate of the present invention comprises two polypeptide sequences that are at least about 80%, 85%, 90%, 95%, 96%, 97%, 98%, 99% or 100% identical to SEQ ID NO: 106 and SEQ ID NO: 116 or variants thereof that retain functionality. In yet another specific embodiment, the immunoconjugate of the present invention comprises two polypeptide sequences that are at least about 80%, 85%, 90%, 95%, 96%, 97%, 98%, 99% or 100% identical to SEQ ID NO: 108 and SEQ ID NO: 113 or variants thereof that retain functionality. In yet another specific embodiment, the immunoconjugate of the present invention comprises two polypeptide sequences that are at least about 80%, 85%, 90%, 95%, 96%, 97%, 98%, 99% or 100% identical to SEQ ID NO: 109 and SEQ ID NO: 112 or variants thereof that retain functionality. In yet another specific embodiment, the immunoconjugate of the present invention comprises two polypeptide sequences that are at least about 80%, 85%, 90%, 95%, 96%, 97%, 98%, 99% or 100% identical to SEQ ID NO: 110 and SEQ ID NO: 112 or variants thereof that retain functionality. In yet another specific embodiment, the immunoconjugate of the present invention comprises two polypeptide sequences that are at least about 80%, 85%, 90%, 95%, 96%, 97%, 98%, 99% or 100% identical to SEQ ID NO: 111 and SEQ ID NO: 112 or variants thereof that retain functionality.

In one embodiment, the immunoconjugate comprises at least one, typically two or more antigen binding moieties that are specific for the Melanoma Chondroitin Sulfate Proteoglycan (MCSP). In another embodiment, the immunoconjugate comprises a polypeptide sequence wherein a first Fab heavy chain specific for MCSP shares a carboxy-terminal peptide bond with an IL-2 molecule, an IL-12 molecule, an IFN a molecule or a GM-CSF molecule, which in turn shares a carboxy-terminal peptide bond with a second Fab heavy chain specific for MCSP. In yet another embodiment, the immunoconjugate comprises a polypeptide sequence wherein a first Fab heavy chain specific for MCSP shares a carboxy-terminal peptide bond with an IL-2 molecule, which in turn shares a carboxy-terminal peptide bond with a second Fab heavy chain specific for MCSP. In a specific embodiment, the antigen binding moieties of the immunoconjugate comprise a heavy chain variable region sequence that is at least about 80%, 85%, 90%, 95%, 96%, 97%, 98%, 99% or 100% identical to the sequence of either SEQ ID NO: 86 or SEQ ID NO: 88 or variants thereof that retain functionality. In another specific embodiment, the antigen binding moieties of the immunoconjugate comprise a light chain variable region sequence that is at least about 80%, 85%, 90%, 95%, 96%, 97%, 98%, 99% or 100% identical to the sequence of either SEQ ID NO: 87 or SEQ ID NO: 90 or variants thereof that retain functionality. In a more specific embodiment, the antigen binding moieties of the immunoconjugate comprise a heavy chain variable region sequence that is at least about 80%, 85%, 90%, 95%, 96%, 97%, 98%, 99% or 100% identical to the sequence of either SEQ ID NO: 86 or SEQ ID NO: 88, or variants thereof that retain functionality, and a light chain variable region sequence that is at least about 80%, 85%, 90%, 95%, 96%, 97%, 98%, 99% or 100% identical to the sequence of either SEQ ID NO: 87 or SEQ ID NO: 90, or variants thereof that retain functionality. In a more specific embodiment, the antigen binding moieties of the immunoconjugate comprise a heavy chain variable region sequence that is at least about 80%, 85%, 90%, 95%, 96%, 97%, 98%, 99% or 100% identical to the sequence of SEQ ID NO: 86, and a light chain variable region sequence that is at least about 80%, 85%, 90%, 95%, 96%, 97%, 98%, 99% or 100% identical to the sequence of SEQ ID NO: 87. In another specific embodiment, the antigen binding moieties of the immunoconjugate comprise a heavy chain variable region sequence that is at least about 80%, 85%, 90%, 95%, 96%, 97%, 98%, 99% or 100% identical to the sequence of SEQ ID NO: 88, and a light chain variable region sequence that is at least about 80%, 85%, 90%, 95%, 96%, 97%, 98%, 99% or 100% identical to the sequence of SEQ ID NO: 87. In another specific embodiment, the immunoconjugate of the invention comprises a polypeptide sequence that is at least about 80%, 85%, 90%, 95%, 96%, 97%, 98%, 99% or 100% identical to either SEQ ID NO: 123 or SEQ ID NO: 125, or variants thereof that retain functionality. In another specific embodiment, the immunoconjugate of the invention comprises a polypeptide sequence that is at least about 80%, 85%, 90%, 95%, 96%, 97%, 98%, 99% or 100% identical to either SEQ ID NO: 124 or SEQ ID NO: 127, or variants thereof that retain functionality. In a more specific embodiment, the immunoconjugate of the present invention comprises a polypeptide sequence that is at least about 80%, 85%, 90%, 95%, 96%, 97%, 98%, 99% or 100% identical to either SEQ ID NO: 123 or SEQ ID NO: 125 or variants thereof that retain functionality, and a polypeptide sequence that is at least about 80%, 85%, 90%, 95%, 96%, 97%, 98%, 99% or 100% identical to either SEQ ID NO: 124 or SEQ ID NO: 127, or variants thereof that retain functionality. In another specific embodiment, the immunoconjugate of the present invention comprises a polypeptide sequence that is at least about 80%, 85%, 90%, 95%, 96%, 97%, 98%, 99% or 100% identical to SEQ ID NO: 123 or variants thereof that retain functionality, and a polypeptide sequence that is at least about 80%, 85%, 90%, 95%, 96%, 97%, 98%, 99% or 100% identical to SEQ ID NO: 124 or variants thereof that retain functionality. In another specific embodiment, the immunoconjugate of the present invention comprises a polypeptide sequence that is at least about 80%, 85%, 90%, 95%, 96%, 97%, 98%, 99% or 100% identical to SEQ ID NO: 125 or variants thereof that retain functionality, and a polypeptide sequence that is at least about 80%, 85%, 90%, 95%, 96%, 97%, 98%, 99% or 100% identical to SEQ ID NO: 124 or variants thereof that retain functionality.

In one embodiment, the immunoconjugate comprises at least one, typically two or more antigen binding moieties that are specific for the Carcinoembryonic Antigen (CEA). In another embodiment, the immunoconjugate comprises a polypeptide sequence wherein a first Fab heavy chain specific for CEA shares a carboxy-terminal peptide bond with an IL-2 molecule, an IL-12 molecule, an IFN a molecule or a GM-CSF molecule, which in turn shares a carboxy-terminal peptide bond with a second Fab heavy chain specific for CEA. In yet another embodiment, the immunoconjugate comprises a polypeptide sequence wherein a first Fab heavy chain specific for CEA shares a carboxy-terminal peptide bond with an IL-2 molecule, which in turn shares a carboxy-terminal peptide bond with a second Fab heavy chain specific for CEA. In a specific embodiment, the antigen binding moieties of the immunoconjugate comprise a heavy chain variable region sequence that is at least about 80%, 85%, 90%, 95%, 96%, 97%, 98%, 99% or 100% identical to the sequence of SEQ ID NO: 154 or a variant thereof that retains functionality. In another specific embodiment, the antigen binding moieties of the immunoconjugate comprise a light chain variable region sequence that is at least about 80%, 85%, 90%, 95%, 96%, 97%, 98%, 99% or 100% identical to the sequence of SEQ ID NO: 155 or a variant thereof that retains functionality. In a more specific embodiment, the antigen binding moieties of the immunoconjugate comprise a heavy chain variable region sequence that is at least about 80%, 85%, 90%, 95%, 96%, 97%, 98%, 99% or 100% identical to the sequence of SEQ ID NO: 154, or a variant thereof that retains functionality, and a light chain variable region sequence that is at least about 80%, 85%, 90%, 95%, 96%, 97%, 98%, 99% or 100% identical to the sequence of SEQ ID NO: 155, or a variant thereof that retains functionality.

Antigen-binding moieties of the invention include those that comprise sequences that are at least about 80%, 85%, 90%, 95%, 96%, 97%, 98%, 99%, or 100% identical to the sequences set forth in SEQ ID NOs 3-127, including functional fragments or variants thereof. The invention also encompasses antigen-binding moieties comprising sequences of SEQ ID NOs 3-127 with conservative amino acid substitutions. It is understood that in the sequences of SEQ ID NOs 91, 93, 94, 95, 96, 102, 103, 104, 105, 106, 108, 109, 110, 111, 117, 118, 119, 123 and 125, the sequence of human IL-2 (see SEQ ID NO: 1) may be replaced by the sequence of an IL-2 analogon, particularly the mutant IL-2 described herein (see SEQ ID NO: 2).

Effector Moieties of Immunoconjugates

The effector moieties for use in the invention are generally polypeptides that influence cellular activity, for example, through signal transduction pathways. Accordingly, the effector moiety of the immunoconjugate useful in the invention can be associated with receptor-mediated signaling that transmits a signal from outside the cell membrane to modulate a response within the cell. For example, an effector moiety of the immunoconjugate can be a cytokine. In a particular embodiment, the effector moiety is a single-chain effector moiety as defined herein. In one embodiment, one or more effector moieties, typically single-chain effector moieties, of the immunoconjugates of the invention are cytokines selected from the group consisting of: IL-2, GM-CSF, IFN-α, and IL-12. In one embodiment the effector moiety is IL-2. In another embodiment, one or more single-chain effector moieties of the immunoconjugates are cytokines selected from the group consisting of: IL-8, MIP-1α, MIP-1β, and TGF-β.

In one embodiment, the effector moiety, particularly a single-chain effector moiety, of the immunoconjugate is IL-2. In a specific embodiment, the IL-2 effector moiety can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In certain embodiments, the IL-2 effector moiety is a mutant IL-2 effector moiety comprising at least one amino acid mutation that reduces or abolishes the affinity of the mutant IL-2 effector moiety to the α-subunit of the IL-2 receptor (also known as CD25) but preserves the affinity of the mutant IL-2 effector moiety to the intermediate-affinity IL-2 receptor (consisting of the β- and γ-subunits of the IL-2 receptor), compared to the non-mutated IL-2 effector moiety. In one embodiment the amino acid mutations are amino acid substitutions. In a specific embodiment, the mutant IL-2 effector moiety comprises one, two or three amino acid substitutions at one, two or three position(s) selected from the positions corresponding to residue 42, 45, and 72 of human IL-2. In a more specific embodiment, the mutant IL-2 effector moiety comprises three amino acid substitutions at the positions corresponding to residue 42, 45 and 72 of human IL-2. In an even more specific embodiment, the mutant IL-2 effector moiety is human IL-2 comprising the amino acid substitutions F42A, Y45A and L72G. In one embodiment the mutant IL-2 effector moiety additionally comprises an amino acid mutation at a position corresponding to position 3 of human IL-2, which eliminates the O-glycosylation site of IL-2. Particularly said additional amino acid mutation is an amino acid substitution replacing a threonine residue by an alanine residue. The sequence of a quadruple mutant (QM) IL-2 comprising the amino acid substitutions T3A, F42A, Y45A and L72G is shown in SEQ ID NO: 2. Suitable mutant IL-2 molecules are described in more detail in European Patent Application number EP11153964.9.

Mutant IL-2 molecules useful as effector moieties in the immunoconjugates can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. In this regard, the nucleotide sequence of native IL-2 has been described by Taniguchi et al. (Nature 302, 305-10 (1983)) and nucleic acid encoding human IL-2 is available from public depositories such as the American Type Culture Collection (Rockville Md.). An exemplary sequence of human IL-2 is shown in SEQ ID NO: 1. Substitution or insertion may involve natural as well as non-natural amino acid residues. Amino acid modification includes well known methods of chemical modification such as the addition or removal of glycosylation sites or carbohydrate attachments, and the like.

In one embodiment, the effector moiety, particularly a single-chain effector moiety, of the immunoconjugate is GM-CSF. In a specific embodiment, the GM-CSF effector moiety can elicit proliferation and/or differentiation in a granulocyte, a monocyte or a dendritic cell. In one embodiment, the effector moiety, particularly a single-chain effector moiety, of the immunoconjugate is IFN-α. In a specific embodiment, the IFN-α effector moiety can elicit one or more of the cellular responses selected from the group consisting of: inhibiting viral replication in a virus-infected cell, and upregulating the expression of major histocompatibility complex I (MHC I). In another specific embodiment, the IFN-α effector moiety can inhibit proliferation in a tumor cell. In one embodiment, the effector moiety, particularly a single-chain effector moiety, of the immunoconjugate is IL-12. In a specific embodiment, the IL-12 effector moiety can elicit one or more of the cellular responses selected from the group consisting of: proliferation in a NK cell, differentiation in a NK cell, proliferation in a T cell, and differentiation in a T cell. In one embodiment, the effector moiety, particularly a single-chain effector moiety, of the immunoconjugate is IL-8. In a specific embodiment, the IL-8 effector moiety can elicit chemotaxis in neutrophils. In one embodiment, the effector moiety, particularly a single-chain effector moiety, of the immunoconjugate, is MIP-1α. In a specific embodiment, the MIP-1α effector moiety can elicit chemotaxis in monocytes and T lymphocyte cells. In one embodiment, the effector moiety, particularly a single-chain effector moiety, of the immunoconjugate is MIP-1β. In a specific embodiment, the MIP-1β effector moiety can elicit chemotaxis in monocytes and T lymphocyte cells. In one embodiment, the effector moiety, particularly a single-chain effector moiety, of the immunoconjugate is TGF-β. In a specific embodiment, the TGF-β effector moiety can elicit one or more of the cellular responses selected from the group consisting of: chemotaxis in monocytes, chemotaxis in macrophages, upregulation of IL-1 expression in activated macrophages, and upregulation of IgA expression in activated B cells.

Antibodies

Antibodies useful in the present invention include antibodies or antibody fragments that bind to a specific antigenic determinant, for example a specific tumor cell antigen, and comprise an Fc region. In certain embodiments the antibody is directed to an antigen presented on a tumor cell. Particular target antigens of the antibodies useful in the present invention include antigens expressed on the surface of tumor cells, including, but not limited to, cell surface receptors such as epidermal growth factor receptor (EGFR), insulin-like growth factor receptors (IGFR) and platelet-derived growth factor receptors (PDGFR), prostate specific membrane antigen (PSMA), carcinoembryonic antigen (CEA), dipeptidyl peptidase IV (CD26, DPPIV), FAP, HER2/neu, HER-3, E-cadherin, CD20, melanoma-associated chondroitin sulfate proteoglycan (MCSP), c-Met, CUB domain-containing protein-1 (CDCP1), and squamous cell carcinoma antigen (SCCA).

In a specific embodiment the antibody is directed to an antigen selected from the group of CD20, Epidermal Growth Factor Receptor (EGFR), HER2, HER3, Insulin-like Growth Factor 1 Receptor (IGF-1R), Carcinoembryonic Antigen (CEA), c-Met, CUB domain-containing protein-1 (CDCP1), and Melanoma-associated Chondroitin Sulfate Proteoglycan (MCSP). In one embodiment, the antibody a multispecific antibody directed to two or more antigens selected from the group of CD20, Epidermal Growth Factor Receptor (EGFR), HER2, HER3, Insulin-like Growth Factor 1 Receptor (IGF-1R), Carcinoembryonic Antigen (CEA), c-Met, CUB domain-containing protein-1 (CDCP1), and Melanoma-associated Chondroitin Sulfate Proteoglycan (MCSP).

Specific anti-CD20 antibodies useful in the present invention are humanized, IgG-class Type II anti-CD20 antibodies, having the binding specificity of the murine B-Ly1 antibody (Poppema and Visser, Biotest Bulletin 3, 131-139 (1987)). Particularly useful is a humanized, IgG-class Type II anti-CD20 antibody, comprising
a) in the heavy chain variable domain a CDR1 of SEQ ID NO: 128, a CDR2 of SEQ ID NO: 129, and a CDR3 of SEQ ID NO: 130, and
b) in the light chain variable domain a CDR1 of SEQ ID NO: 131, a CDR2 of SEQ ID NO: 132, and a CDR3 of SEQ ID NO: 133.

Particularly, the heavy chain variable region framework regions (FRs) FR1, FR2, and FR3 of said antibody are human FR sequences encoded by the VH1_{—}10 human germ-line sequence, the heavy chain variable region FR4 of said antibody is a human FR sequence encoded by the JH4 human germ-line sequence, the light chain variable region FRs FR1, FR2, and FR3 of said antibody are human FR sequences encoded by the VK_{—}2_{—}40 human germ-line sequence, and the light chain variable region FR4 of said antibody is a human FR sequence encoded by the JK4 human germ-line sequence.

A more particular anti-CD20 antibody which is useful in the present invention comprises the heavy chain variable domain of SEQ ID NO: 134 and the light chain variable domain of SEQ ID NO: 135.

Such anti-CD20 antibodies are described in WO 2005/044859, which is incorporated herein by reference in its entirety.

Specific anti-EGFR antibodies useful in the present invention are humanized, IgG-class antibodies, having the binding specificity of the rat ICR62 antibody (Modjtahedi et al., Br J Cancer 67, 247-253 (1993)). Particularly useful is a humanized, IgG-class anti-EGFR antibody, comprising
a) in the heavy chain variable domain a CDR1 of SEQ ID NO: 136, a CDR2 of SEQ ID NO: 137, and a CDR3 of SEQ ID NO: 138, and
b) in the light chain variable domain a CDR1 of SEQ ID NO: 139, a CDR2 of SEQ ID NO: 140, and a CDR3 of SEQ ID NO: 141.

A more particular anti-EGFR antibody which is useful in the invention comprises the heavy chain variable domain of SEQ ID NO: 142 and the light chain variable domain of SEQ ID NO: 143.

Such anti-EGFR antibodies are described in WO 2006/082515 and WO 2008/017963, each of which is incorporated herein by reference in its entirety.

Other suitable humanized IgG-class anti-EGFR antibodies useful for the invention include cetuximab/IMC-C225 (Erbitux®, described in Goldstein et al., Clin Cancer Res 1, 1311-1318 (1995)), panitumumab/ABX-EGF (Vectibix®, described in Yang et al., Cancer Res 59, 1236-1243 (1999), Yang et al., Critical Reviews in Oncology/Hematology 38, 17-23 (2001)), nimotuzumab/h-R3 (TheraCim®, described in Mateo et al., Immunotechnology 3, 71-81 (1997); Crombet-Ramos et al., Int J Cancer 101, 567-575 (2002), Boland & Bebb, Expert Opin Biol Ther 9, 1199-1206 (2009)), matuzumab/EMD 72000 (described in Bier et al., Cancer Immunol Immunother 46, 167-173 (1998), Kim, Curr Opin Mol Ther 6, 96-103 (2004)), and zalutumumab/2F8 (described in Bleeker et al., J Immunol 173, 4699-4707 (2004), Lammerts van Bueren, PNAS 105, 6109-6114 (2008)).

Specific anti-IGF-1R antibodies useful in the present invention are described in WO 2005/005635 and WO 2008/077546, the entire content of each of which is incorporated herein by reference, and inhibit the binding of insulin-like growth factor-1 (IGF-1) and insulin-like growth factor-2 (IGF-2) to insulin-like growth factor-1 receptor (IGF-1R).

The anti-IGF-1R antibodies useful for the invention are preferably monoclonal antibodies and, in addition, chimeric antibodies (human constant domain), humanized antibodies and especially preferably fully human antibodies. Particular anti-IGF-1R antibodies useful for the invention bind to human IGF-1R in competition to antibody 18, i.e. they bind to the same epitope of IGF-1R as antibody 18, which is described in WO 2005/005635. Particular anti-IGF-1R antibodies are further characterized by an affinity to IGF-1R of 10⁻⁸ M (K_{D}) or less, particularly of about 10⁻⁹ to 10⁻¹³ M, and preferably show no detectable concentration-dependent inhibition of insulin binding to the insulin receptor.

Particular anti-IGF-1R antibodies useful for the invention comprise complementarity determining regions (CDRs) having the following sequences:
a) an antibody heavy chain comprising as CDRs CDR1, CDR2 and CDR3 of SEQ ID NO: 144 or 146;
b) an antibody light chain comprising as CDRs CDR1, CDR2 and CDR3 of SEQ ID NO: 145 or 147.

Particularly, the anti-IGF-1R antibodies useful for the invention comprise an antibody heavy chain variable domain amino acid sequence of SEQ ID NO: 41 and an antibody light chain variable domain amino acid sequence of SEQ ID NO: 42, or an antibody heavy chain variable domain amino acid sequence of SEQ ID NO: 43 and an antibody light chain variable domain amino acid sequence of SEQ ID NO: 44.

Particular anti-IGF-1R antibodies useful for the invention are obtainable from the hybridoma cell lines <IGF-1R> HUMAB-Clone 18 and <IGF-1R> HUMAB-Clone 22, which are deposited with Deutsche Sammlung von Mikroorganismen and Zellkulturen GmbH (DSMZ), Germany, under deposition numbers DSM ACC 2587 and DSM ACC 2594, respectively.

Other suitable anti-IGF-1R antibodies useful for the invention are e.g. the fully human IgG1 mAb cixutumumab/IMC-A12 (described in Burtrum et al., Cancer Res 63, 8912-21 (2003); Rowinsky et al., Clin Cancer Res 13, 5549s-5555s (2007), the fully human IgG1 mAb AMG-479 (described in Beltran et al., Mol Cancer Ther 8, 1095-1105 (2009); Tolcher et al., J Clin Oncol 27, 5800-7 (2009)), the humanized IgG1 mAb MK-0646/h7C10 (described in Goetsch et al., Int JCancer 113, 316-28 (2005); Broussas et al., Int J Cancer 124, 2281-93 (2009); Hidalgo et al., J Clin Oncol 26, abstract 3520 (2008); Atzori et al., J Clin Oncol 26, abstract 3519 (2008)), the humanized IgG1 mAb AVE1642 (described in Descamps et al., Br J Cancer 100, 366-9 (2009); Tolcher et al., J Clin Oncol 26, abstract 3582 (2008); Moreau et al., Blood 110, abstract 1166 (2007); Maloney et al., Cancer Res 63, 5073-83 (2003)), the fully human IgG2 mAb figitumumab/CP-751,871 (Cohen et al., Clin Cancer Res 11, 2063-73 (2005); Haluska et al., Clin Cancer Res 13, 5834-40 (2007); Lacy et al., J Clin Oncol 26, 3196-203 (2008); Gualberto & Karp, Clin Lung Cancer 10, 273-80 (2009), the fully human IgG1 mAb SCH-717454 (described in WO 2008/076257 or Kolb et al., Pediatr Blood Cancer 50, 1190-7 (2008)), the 2.13.2. mAb (described in U.S. Pat. No. 7,037,498 (WO 2002/053596)) or the fully human IgG4 mAb BIIB022.

Specific anti-CEA antibodies useful in the present invention are humanized, IgG-class antibodies, having the binding specificity of the murine PR1A3 antibody (Richman and Bodmer, Int J Cancer 39, 317-328 (1987)). Particularly useful is a humanized, IgG-class anti-CEA antibody, comprising
a) in the heavy chain variable domain a CDR1 of SEQ ID NO: 148, a CDR2 of SEQ ID NO: 149, and a CDR3 of SEQ ID NO: 150, and
b) in the light chain variable domain a CDR1 of SEQ ID NO: 151, a CDR2 of SEQ ID NO: 152, and a CDR3 of SEQ ID NO: 153.

A more particular anti-CEA antibody which is useful in the invention comprises the heavy chain variable domain of SEQ ID NO: 154 and the light chain variable domain of SEQ ID NO: 155.

Such anti-CEA antibodies are described in PCT publication number WO 2011/023787, which is incorporated herein by reference in its entirety.

Specific anti-HER3 antibodies that are useful in the present invention are humanized, IgG-class antibodies, such as the Mab 205.10.1, Mab 205.10.2 and Mab 205.10.3, particularly Mab 205.10.2, described in PCT publication number WO 2011/076683.

Specific anti-CDCP1-antibodies that are useful in the present invention are humanized, IgG-class antibodies derived from the CUB4 antibody (deposition number DSM ACC 2551 (DSMZ), as described in PCT publication number WO 2011/023389.

Exemplary anti-MCSP antibodies that can be used in the present invention are described e.g. in WO 2006/100582.

In one embodiment the antibody is a full-length antibody of the IgG-class. In a particular embodiment, the antibody is an IgG1 antibody. In one embodiment, the antibody comprises a human Fc region, more particularly a human IgG Fc region, most particularly a human IgG1 Fc region. The antibodies useful in the invention, such as the anti-IGF-1R, anti-EGFR and anti-CD20 antibodies described above, may comprise a human Ig gamma-1 heavy chain constant region, as set forth in SEQ ID NO: 156 (i.e. the antibodies are of human IgG1 subclass).

The antibodies useful in the present invention are engineered to have increased effector function, compared to a non-engineered antibody. In one embodiment the antibody engineered to have increased effector function has at least 2-fold, at least 10-fold or even at least 100-fold increased effector function, compared to a corresponding non-engineered antibody. The increased effector function can include, but is not limited to, one or more of the following: increased Fc receptor binding, increased C1q binding and complement dependent cytotoxicity (CDC), increased antibody-dependent cell-mediated cytotoxicity (ADCC), increased antibody-dependent cellular phagocytosis (ADCP), increased cytokine secretion, increased immune complex-mediated antigen uptake by antigen-presenting cells, increased binding to NK cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming.

In one embodiment the increased effector function one or more selected from the group of increased Fc receptor binding, increased CDC, increased ADCC, increased ADCP, and increased cytokine secretion. In one embodiment the increased effector function is increased binding to an activating Fc receptor. In one such embodiment the binding affinity to the activating Fc receptor is increased at least 2-fold, particularly at least 10-fold, compared to the binding affinity of a corresponding non-engineered antibody. In a specific embodiment the activating Fc receptor is selected from the group of FcγRIIIa, FcγRI, and FcγRIIa. In one embodiment the activating Fc receptor is FcγRIIIa. In another embodiment the increased effector function is increased ADCC. In one such embodiment the ADCC is increased at least 10-fold, particularly at least 100-fold, compared to the ADCC mediated by a corresponding non-engineered antibody. In yet another embodiment the increased effector function is increased binding to an activating Fc receptor and increased ADCC.

Increased effector function can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998). Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. According to a particular embodiment, binding affinity to an activating Fc receptor is measured by surface plasmon resonance using a BIACORE® T100 machine (GE Healthcare) at 25° C.

Alternatively, binding affinity of antibodies for Fc receptors may be evaluated using cell lines known to express particular Fc receptors, such as NK cells expressing FcγIIIa receptor. Clq binding assays may also be carried out to determine whether the antibody is able to bind Clq and hence has CDC activity. See e.g., Clq and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Increased effector function may result e.g. from glycoengineering of the Fc region or the introduction of amino acid mutations in the Fc region of the antibody. In one embodiment the antibody is engineered by introduction of one or more amino acid mutations in the Fc region. In a specific embodiment the amino acid mutations are amino acid substitutions. In an even more specific embodiment the amino acid substitutions are at positions 298, 333, and/or 334 of the Fc region (EU numbering of residues). Further suitable amino acid mutations are described e.g. in Shields et al., J Biol Chem 9(2), 6591-6604 (2001); U.S. Pat. No. 6,737,056; WO 2004/063351 and WO 2004/099249. Mutant Fc regions can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

In another embodiment the antibody is engineered by modification of the glycosylation in the Fc region. In a specific embodiment the antibody is engineered to have an increased proportion of non-fucosylated oligosaccharides in the Fc region as compared to a non-engineered antibody. An increased proportion of non-fucosylated oligosaccharides in the Fc region of an antibody results in the antibody having increased effector function, in particular increased ADCC.

In a more specific embodiment, at least about 20%, about 25%, about 30%, about 35%, about 40%, about 45%, about 50%, about 55%, about 60%, about 65%, about 70%, about 75%, about 80%, about 85%, about 90%, about 95%, or about 100%, preferably at least about 50%, more preferably at least about 70%, of the N-linked oligosaccharides in the Fc region of the antibody are non-fucosylated. The non-fucosylated oligosaccharides may be of the hybrid or complex type.

In another specific embodiment the antibody is engineered to have an increased proportion of bisected oligosaccharides in the Fc region as compared to a non-engineered antibody. In a more specific embodiment, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, preferably at least about 50%, more preferably at least about 70%, of the N-linked oligosaccharides in the Fc region of the antibody are bisected. The bisected oligosaccharides may be of the hybrid or complex type.

In yet another specific embodiment the antibody is engineered to have an increased proportion of bisected, non-fucosylated oligosaccharides in the Fc region, as compared to a non-engineered antibody. In a more specific embodiment, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, preferably at least about 15%, more preferably at least about 25%, at least about 35% or at least about 50%, of the N-linked oligosaccharides in the Fc region of the antibody are bisected, non-fucosylated. The bisected, non-fucosylated oligosaccharides may be of the hybrid or complex type.

The oligosaccharide structures in the antibody Fc region can be analysed by methods well known in the art, e.g. by MALDI TOF mass spectrometry as described in Umana et al., Nat Biotechnol 17, 176-180 (1999) or Ferrara et al., Biotechn Bioeng 93, 851-861 (2006). The percentage of non-fucosylated oligosaccharides is the amount of oligosaccharides lacking fucose residues, relative to all oligosaccharides attached to Asn 297 (e.g. complex, hybrid and high mannose structures) and identified in an N-glycosidase F treated sample by MALDI TOF MS. Asn 297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. The percentage of bisected, or bisected non-fucosylated, oligosaccharides is determined analogously.

In one embodiment the antibody is engineered to have modified glycosylation in the Fc region, as compared to a non-engineered antibody, by producing the antibody in a host cell having altered activity of one or more glycosyltransferase. Glycosyltransferases include β(1,4)-N-acetylglucosaminyltransferase III (GnTIII), β(1,4)-galactosyltransferase (GalT), β(1,2)-N-acetylglucosaminyltransferase I (GnTI), β(1,2)-N-acetylglucosaminyltransferase II (GnTII) and α(1,6)-fucosyltransferase. In a specific embodiment the antibody is engineered to have an increased proportion of non-fucosylated oligosaccharides in the Fc region, as compared to a non-engineered antibody, by producing the antibody in a host cell having increased β(1,4)-N-acetylglucosaminyltransferase III (GnTIII) activity. In an even more specific embodiment the host cell additionally has increased α-mannosidase II (ManII) activity. The glycoengineering methodology that can be used for engineering antibodies useful for the present invention has been described in greater detail in Umana et al., Nat Biotechnol 17, 176-180 (1999); Ferrara et al., Biotechn Bioeng 93, 851-861 (2006); WO 99/54342 (U.S. Pat. No. 6,602,684; EP 1071700); WO 2004/065540 (U.S. Pat. Appl. Publ. No. 2004/0241817; EP 1587921), WO 03/011878 (U.S. Pat. Appl. Publ. No. 2003/0175884), the entire content of each of which is incorporated herein by reference in its entirety. Antibodies glycoengineered using this methodology are referred to as GlycoMabs herein.

Generally, any type of cultured cell line, including the cell lines discussed herein, can be used to generate cell lines for the production of anti-TNC A2 antibodies with altered glycosylation pattern. Particular cell lines include CHO cells, BHK cells, NS0 cells, SP2/0 cells, YO myeloma cells, P3X63 mouse myeloma cells, PER cells, PER.C6 cells or hybridoma cells, and other mammalian cells. In certain embodiments, the host cells have been manipulated to express increased levels of one or more polypeptides having β(1,4)-N-acetylglucosaminyltransferase III (GnTIII) activity. In certain embodiments the host cells have been further manipulated to express increased levels of one or more polypeptides having α-mannosidase II (ManII) activity. In a specific embodiment, the polypeptide having GnTIII activity is a fusion polypeptide comprising the catalytic domain of GnTIII and the Golgi localization domain of a heterologous Golgi resident polypeptide. Particularly, said Golgi localization domain is the Golgi localization domain of mannosidase II. Methods for generating such fusion polypeptides and using them to produce antibodies with increased effector functions are disclosed in Ferrara et al., Biotechn Bioeng 93, 851-861 (2006) and WO2004/065540, the entire contents of which are expressly incorporated herein by reference.

The host cells which contain the coding sequence of an antibody useful for the invention and/or the coding sequence of polypeptides having glycosyltransferase activity, and which express the biologically active gene products may be identified e.g. by DNA-DNA or DNA-RNA hybridization; the presence or absence of “marker” gene functions; assessing the level of transcription as measured by the expression of the respective mRNA transcripts in the host cell; or detection of the gene product as measured by immunoassay or by its biological activity—methods which are well known in the art. GnTIII or Man II activity can be detected e.g. by employing a lectin which binds to biosynthetis products of GnTIII or ManII, respectively. An example for such a lectin is the E₄-PHA lectin which binds preferentially to oligosaccharides containing bisecting GlcNAc. Biosynthesis products (i.e. specific oligosaccharide structures) of polypeptides having GnTIII or ManII activity can also be detected by mass spectrometric analysis of oligosaccharides released from glycoproteins produced by cells expressing said polypeptides. Alternatively, a functional assay which measures the increased effector function, e.g. increased Fc receptor binding, mediated by antibodies produced by the cells engineered with the polypeptide having GnTIII or ManII activity may be used.

In another embodiment the antibody is engineered to have an increased proportion of non-fucosylated oligosaccharides in the Fc region, as compared to a non-engineered antibody, by producing the antibody in a host cell having decreased α(1,6)-fucosyltransferase activity. A host cell having decreased α(1,6)-fucosyltransferase activity may be a cell in which the α(1,6)-fucosyltransferase gene has been disrupted or otherwise deactivated, e.g. knocked out (see Yamane-Ohnuki et al., Biotech Bioeng 87, 614 (2004); Kanda et al., Biotechnol Bioeng, 94(4), 680-688 (2006); Niwa et al., J Immunol Methods 306, 151-160 (2006)).

Other examples of cell lines capable of producing defucosylated antibodies include Lec13 CHO cells deficient in protein fucosylation (Ripka et al., Arch Biochem Biophys 249, 533-545 (1986); US Pat. Appl. No. US 2003/0157108; and WO 2004/056312, especially at Example 11). The antibodies useful in the present invention can alternatively be glycoengineered to have reduced fucose residues in the Fc region according to the techniques disclosed in EP 1 176 195 A1, WO 03/084570, WO 03/085119 and U.S. Pat. Appl. Pub. Nos. 2003/0115614, 2004/093621, 2004/110282, 2004/110704, 2004/132140, U.S. Pat. No. 6,946,292 (Kyowa), e.g. by reducing or abolishing the activity of a GDP-fucose transporter protein in the host cells used for antibody production.

Glycoengineered antibodies useful in the invention may also be produced in expression systems that produce modified glycoproteins, such as those taught in WO 03/056914 (GlycoFi, Inc.) or in WO 2004/057002 and WO 2004/024927 (Greenovation).

Recombinant Methods

Methods to produce antibodies and immunoconjugates useful in the invention are well known in the art, and described for example in WO 2011/020783, WO 2005/044859, WO 2006/082515, WO 2008/017963, WO 2005/005635, WO 2008/077546, WO 2011/023787, WO 2011/076683, WO 2011/023389 and WO 2006/100582. Established methods to produce polyclonal antibodies and monoclonal antibodies are also described, e.g., in Harlow and Lane, “Antibodies, a laboratory manual”, Cold Spring Harbor Laboratory, 1988.

Non-naturally occurring antibodies or fragments thereof can be constructed using solid phase-peptide synthesis, can be produced recombinantly (e.g. as described in U.S. Pat. No. 4,816,567) or can be obtained, for example, by screening combinatorial libraries comprising variable heavy chains and variable light chains (see e.g. U.S. Pat. No. 5,969,108 to McCafferty). For recombinant production of immunoconjugates and antibodies useful in the invention, one or more polynucleotide(s) encoding said immunoconjugate or antibody is isolated and inserted into one or more vectors for further cloning and/or expression in a host cell. Such polynucleotides may be readily isolated and sequenced using conventional procedures. Methods which are well known to those skilled in the art can be used to construct expression vectors containing the coding sequence of an antibody or immunoconjugate along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989).

Immunoconjugates useful in the invention may be expressed from a single polynucleotide that encodes the entire immunoconjugate or from multiple (e.g., two or more) polynucleotides that are co-expressed. Polypeptides encoded by polynucleotides that are co-expressed may associate through, e.g., disulfide bonds or other means to form a functional immunoconjugate. For example, the heavy chain portion of an antigen binding moiety may be encoded by a separate polynucleotide from the portion of the immunoconjugate comprising the light chain portion of the antigen binding moiety and the effector moiety. When coexpressed, the heavy chain polypeptides will associate with the light chain polypeptides to form the antigen binding moiety. Alternatively, in another example, the light chain portion of the antigen binding moiety could be encoded by a separate polynucleotide from the portion of the immunoconjugate comprising the heavy chain portion of the antigen binding moiety and the effector moiety.

Host cells suitable for replicating and for supporting expression of recombinant proteins are well known in the art. Such cells may be transfected or transduced as appropriate with the particular expression vector and large quantities of vector containing cells can be grown for seeding large scale fermenters to obtain sufficient quantities of the proteins, e.g. for clinical applications. Suitable host cells include prokaryotic microorganisms, such as E. coli, or various eukaryotic cells, such as Chinese hamster ovary cells (CHO), insect cells, or the like. For example, recombinant proteins may be produced in bacteria in particular when glycosylation is not needed. After expression, the protein may be isolated from the bacterial cell paste in a soluble fraction and can be further purified. In addition to prokaryotes, eukaryotic microbes such as filamentous fungi or yeast are suitable cloning or expression hosts for protein-encoding vectors, including fungi and yeast strains whose glycosylation pathways have been “humanized,” resulting in the production of a protein with a partially or fully human glycosylation pattern. See Gerngross, Nat Biotech 22, 1409-1414 (2004), and Li et al., Nat Biotech 24, 210-215 (2006). Suitable host cells for the expression of (glycosylated) proteins are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177; 6,040,498; 6,420,548; 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney (HEK) line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, particularly a mammalian cell, e.g. a Chinese Hamster Ovary (CHO) cell, a human embryonic kidney (HEK) 293 cell, or lymphoid cell (e.g., YO, NSO, Sp20 cell).

If the antibody and immunoconjugate are intended for human use, chimeric forms of antibodies or antigen binding moieties may be used wherein the antibody constant regions are from a human. A humanized or fully human form of the antibody or antigen binding moiety can also be prepared in accordance with methods well known in the art (see e.g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antibodies or antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the antibodies or antigen-binding moieties useful in the invention to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)).

Antibodies and immunoconjugates prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art.

Pharmaceutical Compositions

In another aspect the invention provides a pharmaceutical composition comprising (a) an immunoconjugate comprising at least one antigen-binding moiety and an effector moiety, and (b) an antibody engineered to have increased effector function, in a pharmaceutically acceptable carrier. These pharmaceutical compositions may be used, e.g., in any of the therapeutic methods described below.

Pharmaceutical compositions of an immunoconjugate and an antibody having increased effector function as described herein are prepared by mixing such immunoconjugate and antibody having the desired degree of purity with one or more optional pharmaceutically acceptable carriers (Remington's Pharmaceutical Sciences 18th edition, Mack Printing Company (1990)), in the form of lyophilized formulations or aqueous solutions. Pharmaceutically acceptable carriers are generally non-toxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The pharmaceutical composition herein may also contain additional active ingredients as necessary for the particular indication being treated, particularly those with complementary activities that do not adversely affect each other. For example, if the disease to be treated is cancer, it may be desirable to further provide one or more anti-cancer agents, e.g. a chemotherapeutic agent, an inhibitor of tumor cell proliferation, or an activator of tumor cell apoptosis. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, Mack Printing Company (1990).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The compositions to be used for in vivo administration are generally sterile. Sterility may be readily accomplished, e.g., by filtration through sterile filtration membranes.

Methods of Treatment

The combination provided herein of (a) an immunoconjugate comprising at least one antigen binding moiety and an effector moiety, and (b) an antibody engineered to have increased effector function, may be used in therapeutic methods.

In one aspect, a combination of (a) an immunoconjugate comprising at least one antigen binding moiety and an effector moiety, and (b) an antibody engineered to have increased effector function, for use as a medicament is provided. In further aspects, a combination of (a) an immunoconjugate comprising at least one antigen binding moiety and an effector moiety, and (b) an antibody engineered to have increased effector function, for use in treating a disease is provided. In certain embodiments, a combination of (a) an immunoconjugate comprising at least one antigen binding moiety and an effector moiety, and (b) an antibody engineered to have increased effector function, for use in a method of treatment is provided. In certain embodiments, the invention provides a combination of (a) an immunoconjugate comprising at least one antigen binding moiety and an effector moiety, and (b) an antibody engineered to have increased effector function, for use in a method of treating an individual having a disease comprising administering to the individual a therapeutically effective amount of the combination. In one such embodiment, the method further comprises administering to the individual a therapeutically effective amount of at least one additional therapeutic agent, e.g., as described below. In further embodiments, the invention provides a combination of (a) an immunoconjugate comprising at least one antigen binding moiety and an effector moiety, and (b) an antibody engineered to have increased effector function, for use in stimulating effector cell function. In certain embodiments, the invention provides a combination of (a) an immunoconjugate comprising at least one antigen binding moiety and an effector moiety, and (b) an antibody engineered to have increased effector function, for use in a method of stimulating effector cell function in an individual comprising administering to the individual an effective amount of the combination to stimulate effector cell function. An “individual” according to any of the above embodiments is a mammal, particularly a human. A “disease” according to any of the above embodiments is a disease treatable by stimulation of effector cell function. In certain embodiments the disease is a cell proliferation disorder, particularly cancer.

In a further aspect, the invention provides for the use of a combination of (a) an immunoconjugate comprising at least one antigen binding moiety and an effector moiety, and (b) an antibody engineered to have increased effector function, in the manufacture or preparation of a medicament. In one embodiment, the medicament is for treatment of a disease. In a further embodiment, the medicament is for use in a method of treating a disease comprising administering to an individual having the disease a therapeutically effective amount of the medicament. In one such embodiment, the method further comprises administering to the individual a therapeutically effective amount of at least one additional therapeutic agent, e.g., as described below. In a further embodiment, the medicament is for stimulating effector cell function. In a further embodiment, the medicament is for use in a method of stimulating effector cell function in an individual comprising administering to the individual an amount of the medicament effective to stimulate effector cell function. An “individual” according to any of the above embodiments is a mammal, particularly a human. A “disease” according to any of the above embodiments is a disease treatable by stimulation of effector cell function. In certain embodiments the disease is a cell proliferation disorder, particularly cancer.

In a further aspect, the invention provides a method for treating a disease. In one embodiment, the method comprises administering to an individual having such disease a therapeutically effective amount of a combination of (a) an immunoconjugate comprising at least one antigen binding moiety and an effector moiety, and (b) an antibody engineered to have increased effector function. In one such embodiment, the method further comprises administering to the individual a therapeutically effective amount of at least one additional therapeutic agent, as described below. An “individual” according to any of the above embodiments is a mammal, particularly a human. A “disease” according to any of the above embodiments is a disease treatable by stimulation of effector cell function. In certain embodiments the disease is a cell proliferation disorder, particularly cancer.

In a further aspect, the invention provides a method for stimulating effector cell function in an individual. In one embodiment, the method comprises administering to the individual an effective amount of a combination of (a) an immunoconjugate comprising at least one antigen binding moiety and an effector moiety, and (b) an antibody engineered to have increased effector function, to stimulate effector cell function. In one embodiment, an “individual” is a mammal, particularly a human.

In a further aspect, the invention provides pharmaceutical composition comprising any of the combinations of (a) an immunoconjugate comprising at least one antigen binding moiety and an effector moiety, and (b) an antibody engineered to have increased effector function provided herein, e.g., for use in any of the above therapeutic methods. In one embodiment, a pharmaceutical composition comprises a combination provided herein, of (a) an immunoconjugate comprising at least one antigen binding moiety and an effector moiety and (b) an antibody engineered to have increased effector function, and a pharmaceutically acceptable carrier. In another embodiment, a pharmaceutical composition comprises any of the combinations provided herein and at least one additional therapeutic agent, e.g., as described below.

According to any of the above embodiments, the disease is a disorder treatable by stimulation of effector cell function. Combinations of the invention are useful in treating disease states where stimulation of the immune system of the host is beneficial, in particular conditions where an enhanced cellular immune response is desirable. These may include disease states where the host immune response is insufficient or deficient. Disease states for which the combinations of the invention can be administered comprise, for example, a tumor or infection where a cellular immune response would be a critical mechanism for specific immunity. Specific disease states for which the combinations of the present invention can be employed include cancer, specifically renal cell carcinoma or melanoma; immune deficiency, specifically in HIV-positive patients, immunosuppressed patients, chronic infection and the like. In certain embodiments the disease is a cell proliferation disorder. In a particular embodiment the disease is cancer, specifically a cancer selected from the group of lung cancer, colorectal cancer, renal cancer, prostate cancer, breast cancer, head and neck cancer, ovarian cancer, brain cancer, lymphoma, leukemia, skin cancer.

Combinations of the invention can be used either alone or together with other agents in a therapy. For instance, a combination of the invention may be co-administered with at least one additional therapeutic agent. In certain embodiments, an additional therapeutic agent is an anti-cancer agent, e.g. a chemotherapeutic agent, an inhibitor of tumor cell proliferation, or an activator of tumor cell apoptosis.

Combination therapies as provided herein encompass administration of the antibody and the immunoconjugate together (where the two or more therapeutic agents are included in the same or separate formulations), and separately, in which case, administration of the antibody can occur prior to, simultaneously, and/or following, administration of the immunoconjugate, additional therapeutic agent and/or adjuvant. Combinations of the invention can also be combined with radiation therapy.

A combination of the invention (and any additional therapeutic agent) can be administered by any suitable route, including parenteral, intrapulmonary, and intranasal, and, if desired for local treatment, intralesional administration. Parenteral infusions include intramuscular, intravenous, intraarterial, intraperitoneal, or subcutaneous administration. The antibody and the immunconjugate may be administered by the same or by different routes. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Combinations of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agents, the method of administration, the scheduling of administration, and other factors known to medical practitioners. The combination need not be, but is optionally formulated with one or more agents currently used to prevent or treat the disorder in question. The effective amount of such other agents depends on the amount of antibody and immunoconjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody and immunoconjugate (when used in the combinations of the invention, optionally together with one or more other additional therapeutic agents) will depend on the type of disease to be treated, the type of antibody and immunoconjugate, the severity and course of the disease, whether the combination is administered for preventive or therapeutic purposes, previous therapy, the patient's clinical history and response to the antibody and/or immunoconjugate, and the discretion of the attending physician. The antibody and the immunoconjugate are suitably administered to the patient at one time or over a series of treatments.

Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. The same considerations with respect to dosage apply to the immunconjugate to be used in the combinations according to the invention. However, other dosage regimens may be useful. The progress of this therapy is easily monitored by conventional techniques and assays.

Articles of Manufacture

In another aspect of the invention, an article of manufacture containing materials useful for the treatment, prevention and/or diagnosis of the disorders described above is provided. The article of manufacture comprises one or more container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody to be used in the combinations of the invention. Another active agent is the immunoconjugate to be used in the combinations of the invention, which may be in the same composition and container like the antibody, or may be provided in a different composition and container. The label or package insert indicates that the composition is used for treating the condition of choice.

In one aspect the invention provides a kit intended for the treatment of a disease, comprising in the same or in separate containers (a) an immunoconjugate comprising at least one antigen binding moiety and an effector moiety, and (b) an antibody engineered to have increased effector function, and optionally further comprising (c) a package insert comprising printed instructions directing the use of the combined treatment as a method for treating the disease. Moreover, the kit may comprise (a) a first container with a composition contained therein, wherein the composition comprises an antibody engineered to have increased effector function; (b) a second container with a composition contained therein, wherein the composition comprises an immunoconjugate comprising at least one antigen binding moiety and an effector moiety; and optionally (c) a third container with a composition contained therein, wherein the composition comprises a further cytotoxic or otherwise therapeutic agent. The kit in this embodiment of the invention may further comprise a package insert indicating that the compositions can be used to treat a particular condition. Alternatively, or additionally, the kit may further comprise a third (or fourth) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

General Methods

Glycoengineereing of the Fc region of an antibody leads to increased binding affinity to human FcγRIII receptors, which in turn translates into enhanced ADCC induction and anti-tumor efficacy. Human FcγRIII receptors are expressed on macrophages, neutrophils, and natural killer (NK), dendritic and γδ T cells. In the mouse, the most widely utilized species for preclinical efficacy testing, murine FcγRIV, the murine homologue of human FcγRIIIa, is present on marcophages and neutrophils but not on NK cells. Therefore, not the full extent of any expected improved efficacy with glycoengineered antibodies is reflected in those models. We have generated a mouse transgenic for human FcγRIIIa (CD16a), exhibiting stable human CD16a expression on murine NK cells in blood, lymphoid tissues and tumors. Moreover, the expression level of human CD16a on unstimulated NK cells in the blood of these transgenic mice mirrors that found in human. We also showed that a down-regulation of human FcγRIIIa on the tumor-associated NK cells after antibody therapy correlates with antitumoral activity. Finally, we showed significantly improved efficacy of glycoengineered antibody treatment in tumor models using this new mouse strain as compared to their human CD16-negative littermates.

### Example 1

A549 Lung Xenograft Model

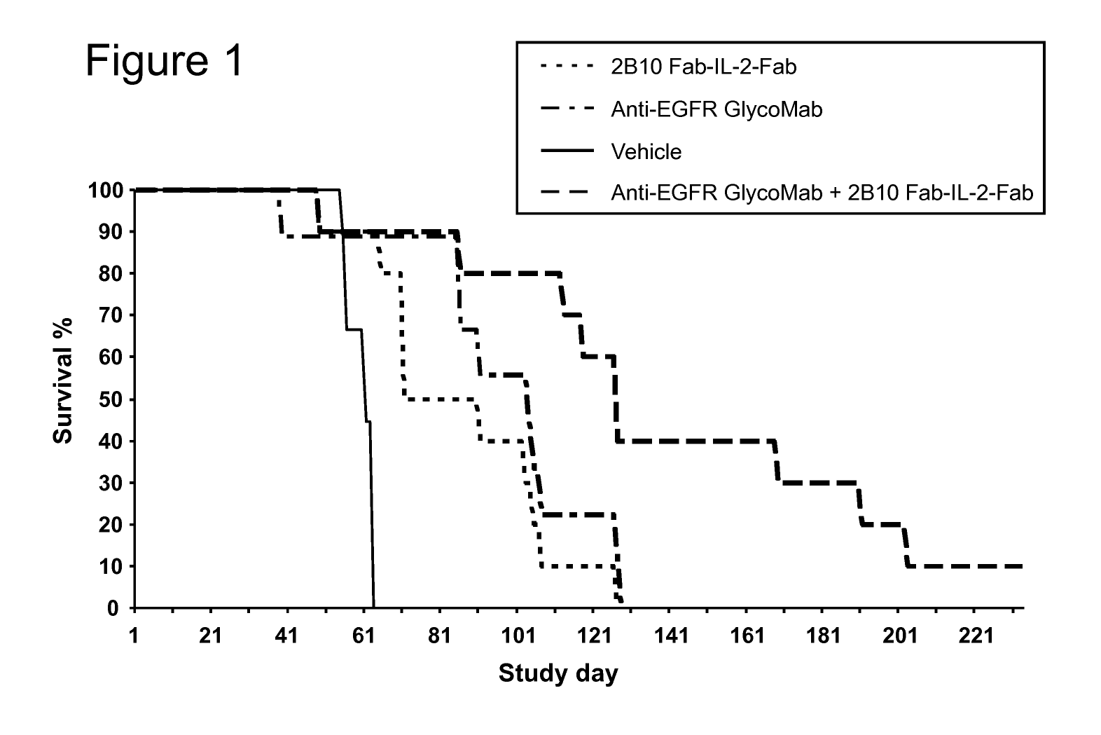

The TNC A2-targeted 2B10 Fab-IL-2-Fab immunoconjugate (SEQ ID NOs 117 and 120) and the anti-EGFR GlycoMab (SEQ ID NOs 142 and 143) were tested in the human non-small cell lung carcinoma (NSCLC) cell line A549, injected i.v. into SCID-human FcγRIII (hCD16) transgenic mice. This tumor model was shown by IHC on fresh frozen tissue to be positive for the A2 domain of Tenascin C. The A549 NSCLC cells were originally obtained from ATCC(CCL-185) and after expansion deposited in the Glycart internal cell bank. The tumor cell line was routinely cultured in DMEM containing 10% FCS (Gibco) at 37° C. in a water-saturated atmosphere at 5% CO₂. Passage 8 was used for transplantation, at a viability of 98%. 5×10⁶ cells per animal were injected i.v. into the tail vein in 200 μl of Aim V cell culture medium (Gibco). Female SCID-FcγRIII mice (GLYCART-RCC), aged 8-9 weeks at the start of the experiment (bred at RCC, Switzerland) were maintained under specific-pathogen-free conditions with daily cycles of 12 h light/12 h darkness according to committed guidelines (GV-Solas; Felasa; TierschG). The experimental study protocol was reviewed and approved by local government (P 2008016). After arrival, animals were maintained for one week to get accustomed to the new environment and for observation. Continuous health monitoring was carried out on a regular basis. Mice were injected i.v. on study day 0 with 5×10⁶ of A549 cells, randomized and weighed. One week after the tumor cell injection, mice were injected i.v. with the 2B10 Fab-IL-2-Fab immunoconjugate twice weekly for three weeks, the anti-EGFR GlycoMab once weekly for three weeks, or the combination of the 2B10 Fab-IL-2-Fab immunoconjugate twice weekly for three weeks and the anti-EGFR GlycoMab once weekly for three weeks. All mice were injected i.v. with 200 μl of the appropriate solution. Doses are specified in Table 2. The mice in the vehicle group were injected with PBS and the treatment group with the 2B10 Fab-IL-2-Fab immunoconjugate or the anti-EGFR GlycoMab or the combination 2B10 Fab-IL-2-Fab immunoconjugate and the anti-EGFR GlycoMab. To obtain the correct amount of immunoconjugate per 200 μl, the stock solutions were diluted with PBS if necessary. FIG. 1 shows that the combination of the 2B10 Fab-IL-2-Fab immunoconjugate and the anti-EGFR-GlycoMab mediated superior efficacy resulting in synergistically enhanced median and overall survival compared to the 2B10 Fab-IL-2-Fab immunoconjugate or the anti-EGFR GlycoMab alone in the hCD16 transgenic SCID mice.

TABLE 2

| | | | Concentration |
|---|---|---|---|
| Compound | Dose/mouse | Formulation buffer | (mg/mL) |
| Anti-EGFR | 625 μg | 20 mM His/HisCl | 9.7 |
| Glycomab | | 240 mM trehalose | (= stock |
| | | 0.02% Tween 20 | solution) |
| | | pH 6.0 | |
| huTNC A2 | 16 μg | 25 mM potassium phosphate, | 1.86 |
| 2B10 | | 125 mM NaCl, | (= stock |
| (G65S) | | 100 mM glycine, | solution) |
| Fab-IL2- | | pH 6.7 | |
| Fab = 2B10 | | | |

### Example 2

LS174T Colorectal Xenograft Model

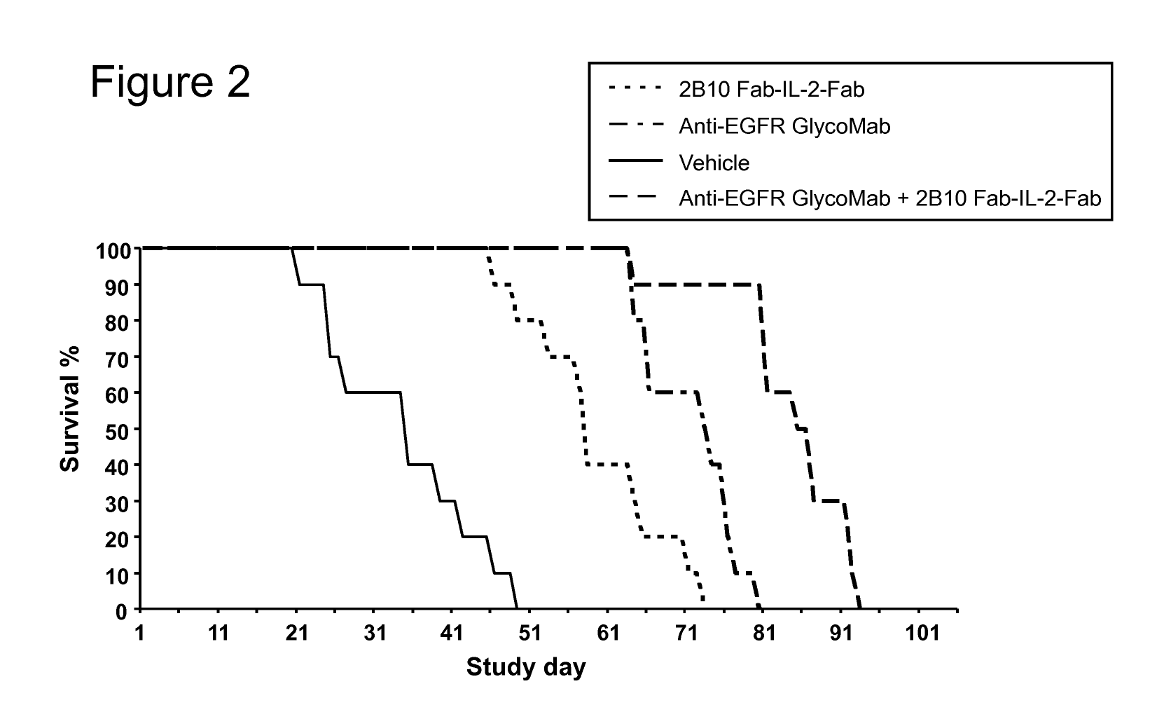

The TNC A2-targeted 2B10 Fab-IL-2-Fab immunoconjugate and the anti-EGFR GlycoMab were tested in the human colorectal LS174T cell line, intrasplenically injected into SCID mice. This tumor model was shown by IHC on fresh frozen tissue to be positive for the A2 domain of Tenascin C. LS174T cells (human colon carcinoma cells) were originally obtained from ECACC (European Collection of Cell Culture) and after expansion deposited in the Glycart internal cell bank. LS174T were cultured in MEM Eagle's medium containing 10% FCS (PAA Laboratories, Austria), 1% Glutamax and 1% MEM Non-Essential Amino Acids (Sigma). The cells were cultured at 37° C. in a water-saturated atmosphere at 5% CO₂. In vitro passage 18 was used for intrasplenic injection, at a viability of 97%. A small incision was made at the left abdominal site of anesthetized SCID mice. Fifty microliters cell suspension (3×10⁶ LS174T cells in AimV medium) was injected through the abdominal wall just under the capsule of the spleen. Skin wounds were closed using clamps. Female SCID mice; aged 8-9 weeks at the start of the experiment (purchased from Taconics, Denmark) were maintained under specific-pathogen-free conditions with daily cycles of 12 h light/12 h darkness according to committed guidelines (GV-Solas; Felasa; TierschG). The experimental study protocol was reviewed and approved by local government (P 2008016). After arrival, animals were maintained for one week to get accustomed to the new environment and for observation. Continuous health monitoring was carried out on a regular basis. Mice were injected intrasplenically on study day 0 with 3×10⁶ LS174T cells, randomized and weighed. One week after the tumor cell injection mice were injected i.v. with the 2B10 Fab-IL-2-Fab immunoconjugate twice weekly for three weeks, the anti-EGFR GlycoMab once weekly for three weeks, or the combination of the 2B10 Fab-IL-2-Fab immunoconjugate twice weekly for three weeks and the anti-EGFR GlycoMab once weekly for three weeks. All mice were injected i.v. with 200 μl of the appropriate solution. Doses are specified in Table 3. The mice in the vehicle group were injected with PBS and the treatment groups with the 2B10 Fab-IL-2-Fab immunoconjugate or the anti-EGFR GlycoMab or the combination 2B10 Fab-IL-2-Fab immunoconjugate and the anti-EGFR GlycoMab. To obtain the proper amount of immunoconjugate per 200 μl, the stock solutions were diluted with PBS when necessary. FIG. 2 shows that the combination of the 2B10 Fab-IL-2-Fab immunoconjugate and the anti-EGFR GlycoMab mediated superior efficacy in terms of enhanced median and overall survival compared to the 2B10 Fab-IL-2-Fab immunoconjugate or the anti-EGFR GlycoMab alone.

TABLE 3

| | | | Concentration |
|---|---|---|---|
| Compound | Dose/mouse | Formulation buffer | (mg/mL) |
| Anti-EGFR | 625 μg | 20 mM His/HisCl | 9.7 |
| Glycomab | | 240 mM trehalose | (= stock |
| | | 0.02% Tween 20 | solution) |
| | | pH 6.0 | |
| huTNC A2 | 16 μg | 25 mM potassium phosphate, | 1.86 |
| 2B10 | | 125 mM NaCl, | (= stock |
| (G65S) | | 100 mM glycine, | solution) |
| Fab-IL-2- | | pH 6.7 | |
| Fab = 2B10 | | | |

### Example 3

ACHN Renal carcinoma Xenograft Model

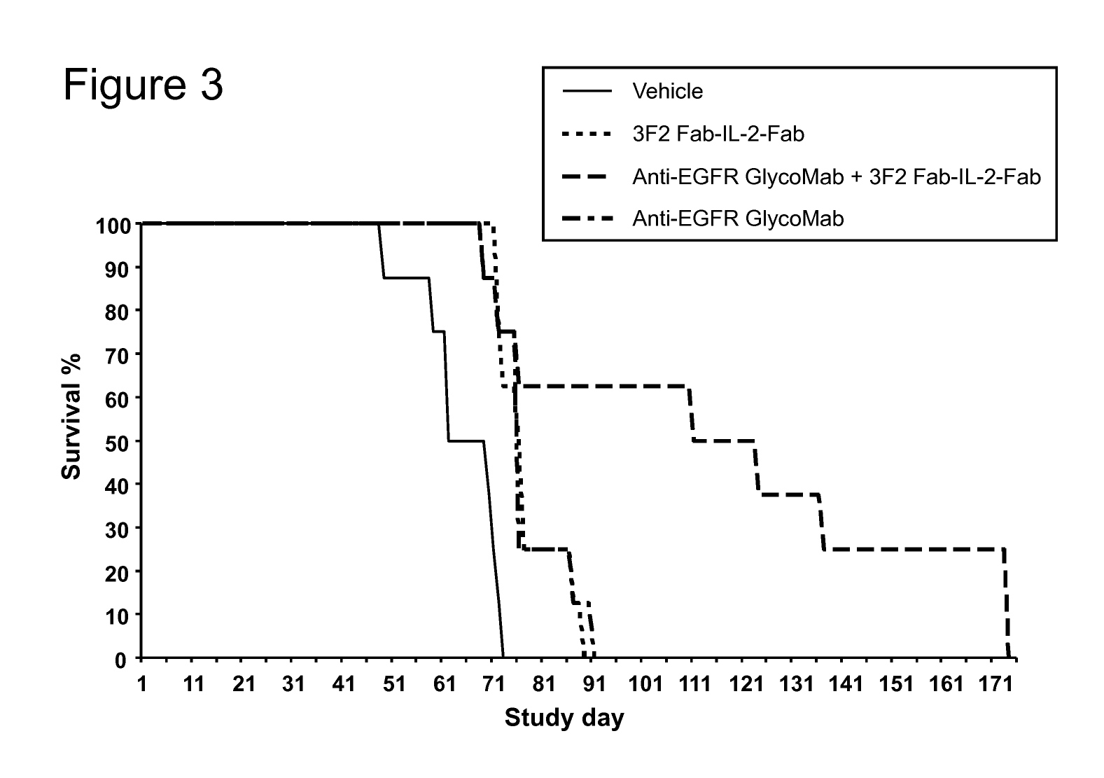

The FAP-targeted 3F2 Fab-IL-2-Fab immunoconjugate (SEQ ID NOs 102 and 112) and the anti-EGFR GlycoMab were tested in the human renal cell line ACHN, intrarenally injected into SCID mice. This tumor model was shown by IHC on fresh frozen tissue to be positive for FAP. ACHN cells (human renal adenocarcinoma cells) were originally obtained from ATCC (American Type Culture Collection) and after expansion deposited in the Glycart internal cell bank. ACHN cells were cultured in DMEM containing 10% FCS, at 37° C. in a water-saturated atmosphere at 5% CO₂. In vitro passage 9 was used for intrarenal injection, at a viability of 97.7%. A small incision (2 cm) was made at the right flank and peritoneal wall of anesthetized SCID mice. Fifty μl cell suspension (1×10⁶ ACHN cells in AimV medium) was injected 2 mm subcapsularly in the kidney. Skin wounds and peritoneal wall were closed using clamps. Female SCID mice; aged 8-9 weeks at the start of the experiment (purchased from Charles River, Sulzfeld, Germany) were maintained under specific-pathogen-free conditions with daily cycles of 12 h light/12 h darkness according to committed guidelines (GV-Solas; Felasa; TierschG). The experimental study protocol was reviewed and approved by local government (P 2008016). After arrival, animals were maintained for one week to get accustomed to new environment and for observation. Continuous health monitoring was carried out on a regular basis. Mice were injected intrarenally on study day 0 with 1×10⁶ ACHN cells, randomized and weighed. One week after the tumor cell injection, mice were injected i.v. with the 3F2 Fab-IL-2-Fab immunoconjugate twice weekly for three weeks, the anti-EGFR GlycoMab once weekly for three weeks, or the combination of the 3F2 Fab-IL-2-Fab immunoconjugate twice weekly for three weeks and the anti-EGFR GlycoMab once weekly for three weeks. All mice were injected i.v. with 200 μl of the appropriate solution. Doses are specified in Table 4. The mice in the vehicle group were injected with PBS and the treatment groups with the 3F2 Fab-IL-2-Fab immunoconjugate, the anti-EGFR GlycoMab or the combination of the 3F2 Fab-IL-2-Fab immunoconjugate and the anti-EGFR GlycoMab. To obtain the correct amount of immunoconjugate per 200 μl, the stock solutions were diluted with PBS if necessary. FIG. 3 shows that the combination of the 3F2 Fab-IL-2-Fab immunoconjugate and the anti-EGFR GlycoMab resulted in synergistically enhanced median and overall survival compared to the 3F2 Fab-IL-2-Fab immunoconjugate and the anti-EGFR GlycoMab alone in SCID mice.

TABLE 4

| | | | Concentration |
|---|---|---|---|
| Compound | Dose/mouse | Formulation buffer | (mg/mL) |
| Anti-EGFR | 625 μg | 20 mM His/HisCl | 9.7 |
| Glycomab | | 240 mM trehalose | (= stock |
| | | 0.02% Tween 20 | solution) |
| | | pH 6.0 | |
| FAP 3F2 | 16 μg | 25 mM potassium phosphate, | 2.46 |
| Fab-IL-2- | | 125 mM NaCl, | (= stock |
| Fab = FAP | | 100 mM glycine, | solution) |
| 3F2 | | pH 6.7 | |

### Example 4

ACHN Renal Carcinoma Xenograft Model

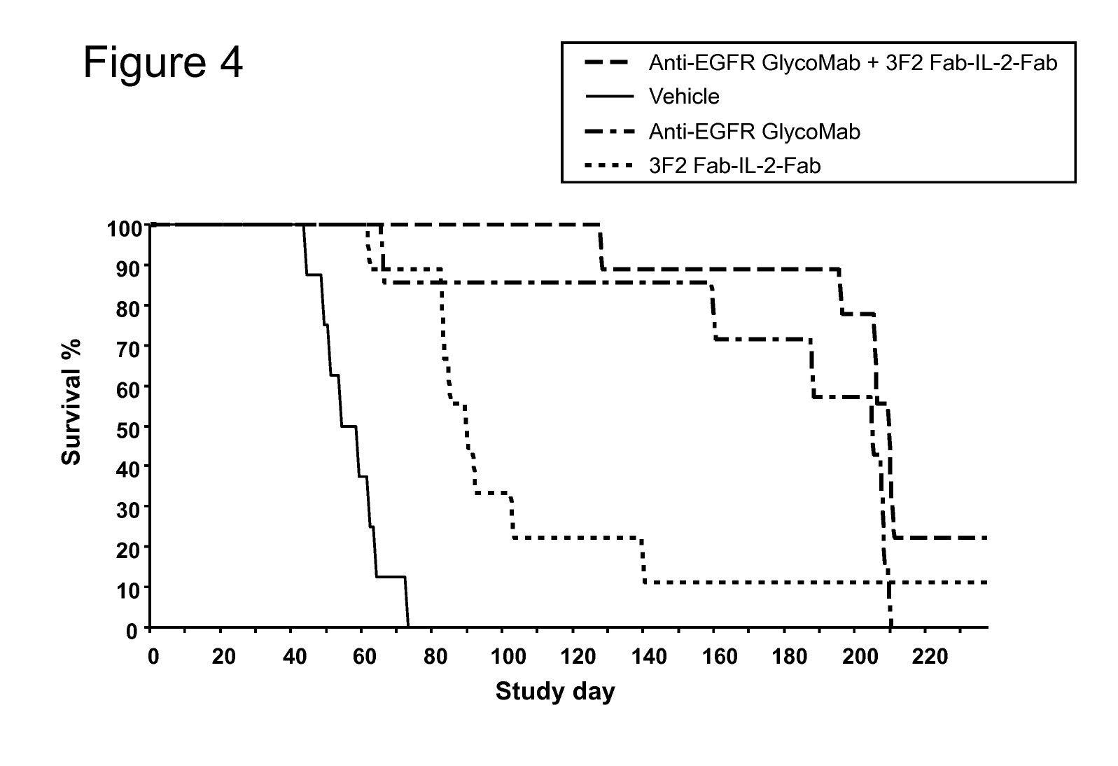

The FAP-targeted 3F2 Fab-IL-2-Fab immunoconjugate and the anti-EGFR GlycoMab were tested in the human renal cell line ACHN, intrarenally injected into SCID-human FcγRIII transgenic mice. This tumor model was shown by IHC on fresh frozen tissue to be positive for FAP. ACHN cells (human renal adenocarcinoma cells) were originally obtained from ATCC (American Type Culture Collection) and after expansion deposited in the Glycart internal cell bank. ACHN cells were cultured in DMEM containing 10% FCS, at 37° C. in a water-saturated atmosphere at 5% CO₂. In vitro passage 11 was used for intrarenal injection, at a viability of 96.7%. A small incision (2 cm) was made at the right flank and peritoneal wall of anesthetized SCID mice. Fifty μl cell suspension (1×10⁶ ACHN cells in AimV medium) was injected 2 mm subcapsularly in the kidney. Skin wounds and peritoneal wall were closed using clamps. Female SCID-FcγRIII mice (GLYCART-RCC), aged 8-9 weeks at the start of the experiment (bred at RCC, Switzerland) were maintained under specific-pathogen-free conditions with daily cycles of 12 h light/12 h darkness according to committed guidelines (GV-Solas; Felasa; TierschG). The experimental study protocol was reviewed and approved by local government (P 2008016). After arrival, animals were maintained for one week to get accustomed to new environment and for observation. Continuous health monitoring was carried out on a regular basis. Mice were injected intrarenally on study day 0 with 1×10⁶ ACHN cells, randomized and weighed. One week after the tumor cell injection, mice were injected i.v. with the 3F2 Fab-IL-2-Fab immunoconjugate twice weekly for three weeks, the anti-EGFR GlycoMab once weekly for three weeks, or the combination of the 3F2 Fab-IL-2-Fab immunoconjugate twice weekly for three weeks and the anti-EGFR GlycoMab once weekly for three weeks. All mice were injected i.v. with 200 μl of the appropriate solution. Doses are specified in Table 5. The mice in the vehicle group were injected with PBS and the treatment groups with the 3F2 Fab-IL-2-Fab immunoconjugate, the anti-EGFR GlycoMab or the combination of the 3F2 Fab-IL-2-Fab immunoconjugate and the anti-EGFR GlycoMab. To obtain the correct amount of immunoconjugate per 200 μl, the stock solutions were diluted with PBS if necessary. FIG. 4 shows that the combination of the 3F2 Fab-IL-2-Fab immunoconjugate and the anti-EGFR GlycoMab mediated superior efficacy in terms of overall survival compared to the 3F2 Fab-IL-2-Fab immunoconjugate or the anti-EGFR GlycoMab alone.

TABLE 5

| | | | Concentration |
|---|---|---|---|
| Compound | Dose/mouse | Formulation buffer | (mg/mL) |
| Anti-EGFR | 625 μg | 20 mM His/HisCl | 9.7 |
| Glycomab | | 240 mM trehalose | (= stock |
| | | 0.02% Tween 20 | solution) |
| | | pH 6.0 | |
| FAP 3F2 | 16 μg | 25 mM potassium phosphate, | 2.46 |
| Fab-IL-2- | | 125 mM NaCl, | (= stock |
| Fab = FAP | | 100 mM glycine, | solution) |
| 3F2 | | pH 6.7 | |

### Example 5

Z138 Mantle Cell Lymphoma Xenograft Model

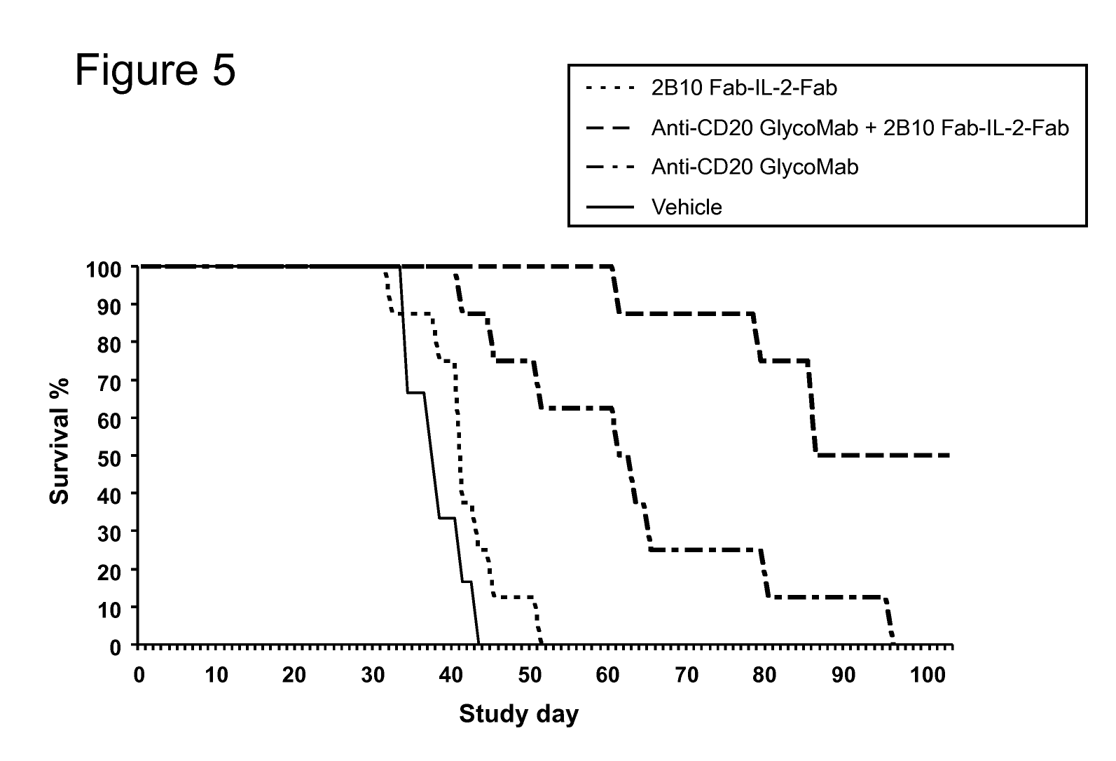

The TNC A2-targeted 2B10 Fab-IL-2-Fab immunoconjugate and the anti-CD20 GlycoMab (SEQ ID NOs 134 and 135) were tested in the human mantle cell lymphoma cell line Z138, injected i.v. into SCID-human FcγRIII transgenic mice. This tumor model was shown by IHC on fresh frozen tissue to be positive for TNC A2. Z138 human mantle cell lymphoma cells were originally obtained from Professor Martin Dyer (MRC Toxicology Unit, Leicester, UK) and after expansion deposited in the Glycart internal cell bank. The tumor cell line was routinely cultured in DMEM containing 10% FCS (Gibco) at 37° C. in a water-saturated atmosphere at 5% CO₂. Passage 18 was used for transplantation, at a viability of 98%. 10×10⁶ cells per animal were injected i.v. into the tail vein in 200 μl of Aim V cell culture medium (Gibco). Female SCID-FcγRIII mice (GLYCART-RCC), aged 8-9 weeks at the start of the experiment (bred at RCC, Switzerland) were maintained under specific-pathogen-free conditions with daily cycles of 12 h light/12 h darkness according to committed guidelines (GV-Solas; Felasa; TierschG). The experimental study protocol was reviewed and approved by local government (P 2008016). After arrival, animals were maintained for one week to get accustomed to the new environment and for observation. Continuous health monitoring was carried out on a regular basis. Mice were injected i.v. on study day 0 with 10×10⁶ Z138 cells, randomized and weighed. One week after the tumor cell injection mice were injected i.v. with the 2B10 Fab-IL-2-Fab immunoconjugate twice weekly for three weeks, the anti-CD20 GlycoMab once weekly for three weeks, or the combination of the 2B10 Fab-IL-2-Fab immunoconjugate twice weekly for three weeks and the anti-CD20 GlycoMab once weekly for three weeks. All mice were injected i.v. with 200 μl of the appropriate solution. Doses are specified in Table 6. The mice in the vehicle group were injected with PBS and the treatment groups with the 2B10 Fab-IL-2-Fab immunoconjugate, the anti-CD20 GlycoMab, or the combination of the 2B10 Fab-IL-2-Fab immunoconjugate and the anti-CD20 GlycoMab. To obtain the correct amount of immunoconjugate per 200 μl, the stock solutions were diluted with PBS when necessary. FIG. 5 shows that the combination the 2B10 Fab-IL-2-Fab immunoconjugate and the anti-CD20 GlycoMab resulted in synergistically enhanced superior efficacy in terms of median and overall survival compared to the 2B10 Fab-IL-2-Fab immunoconjugate or the anti-CD20 GlycoMab alone.

TABLE 6

| | | | Concentration |
|---|---|---|---|
| Compound | Dose/mouse | Formulation buffer | (mg/mL) |
| Anti-CD20 | 625 μg | 20 mM His/HisCl | 10.50 |
| Glycomab | | 140 mM NaCl | (= stock |
| | | 0.02% Tween 20 | solution) |
| | | pH 6.0 | |
| huTNC A2 | 16 μg | 25 mM potassium phosphate, | 1.86 |
| 2B10 | | 125 mM NaCl, | (= stock |
| (G65S) | | 100 mM glycine, | solution) |
| Fab-IL2- | | pH 6.7 | |
| Fab = 2B10 | | | |

### Example 6

ACHN Renal Carcinoma Xenograft Model

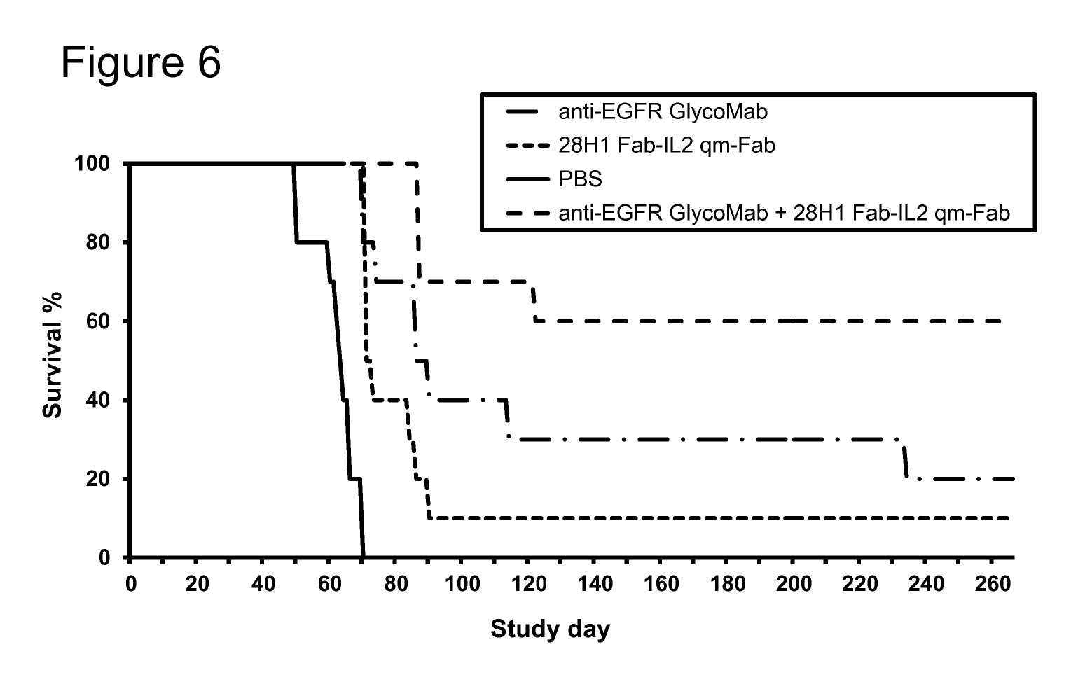

The FAP-targeted 28H1 Fab-IL2-Fab immunoconjugate comprising the IL-2 quadruple mutant (qm) that lacks binding to CD25 (SEQ ID NO: 108 wherein the IL-2 sequence (SEQ ID NO: 1) is replaced by SEQ ID NO: 2; and SEQ ID NO: 113) and the anti-EGFR GlycoMab were tested in the human renal cell line ACHN, intrarenally injected into SCID-human FcγRIII transgenic mice. This tumor model was shown by IHC on fresh frozen tissue to be positive for FAP. ACHN cells (human renal adenocarcinoma cells) were originally obtained from ATCC (American Type Culture Collection) and after expansion deposited in the Glycart internal cell bank. ACHN were cultured in DMEM containing 10% FCS, at 37° C. in a water-saturated atmosphere at 5% CO₂. In vitro passage 18 was used for intrarenal injection, at a viability of 97%. A small incision (2 cm) was made at the right flank and peritoneal wall of anesthetized SCID mice. Fifty μl cell suspension (1×10⁶ ACHN cells in AimV medium) was injected 2 mm subcapsularly in the kidney. Skin wounds and peritoneal wall were closed using clamps. Female SCID-FcγRIII mice (GLYCART-RCC), aged 8-9 weeks at the start of the experiment (bred at RCC, Switzerland) were maintained under specific-pathogen-free conditions with daily cycles of 12 h light/12 h darkness according to committed guidelines (GV-Solas; Felasa; TierschG). The experimental study protocol was reviewed and approved by local government (P 2008016). After arrival, animals were maintained for one week to get accustomed to new environment and for observation. Continuous health monitoring was carried out on a regular basis. Mice were injected intrarenally on study day 0 with 1×10⁶ ACHN cells, randomized and weighed. One week after the tumor cell injection, mice were injected i.v. with the 28H1 Fab-IL-2 qm-Fab immunoconjugate three times a week for three weeks, the anti-EGFR GlycoMab once weekly for three weeks, or the combination of the 28H1 Fab-IL-2 qm-Fab three times a week for three weeks and the anti-EGFR GlycoMab once weekly for three weeks. All mice were injected i.v. with 200 μl of the appropriate solution. Doses are specified in Table 7. The mice in the vehicle group were injected with PBS and the treatment groups with the 28H1 Fab-IL-2 qm-Fab immunoconjugate, the anti-EGFR GlycoMab, or the combination of the 28H1 Fab-IL-2 qm-Fab immunoconjugate and the anti-EGFR GlycoMab. To obtain the proper amount of immunoconjugate per 200 μl, the stock solutions were diluted with PBS when necessary. FIG. 6 shows that the combination of the 28H1 Fab-IL-2 qm-Fab immunoconjugate and the anti-EGFR GlycoMab mediated superior efficacy in terms of enhanced median survival compared to the 28H1 Fab-IL-2 qm-Fab immunoconjugate or the anti-EGFR GlycoMab alone.

TABLE 7

| | | | Concentration |
|---|---|---|---|
| Compound | Dose/mouse | Formulation buffer | (mg/mL) |
| Anti-EGFR | 625 μg | 20 mM His/HisCl | 9.7 |
| Glycomab | | 240 mM trehalose | (= stock |
| | | 0.02% Tween 20 | solution) |
| | | pH 6.0 | |
| FAP 28H1 | 30 μg | 25 mM potassium phosphate, | 2.74 |
| Fab-IL2 qm- | | 125 mM NaCl, | (= stock |
| Fab | | 100 mM glycine, pH 6.7 | solution) |

### Example 7

In Vitro Boosting of NK Cell Killing Capacity and NK Cell IFN-γ Release by IL-2 Immunoconjugates

To determine the effect of immunoconjugates on NK cells, we assessed the killing of tumor cells and IFN-γ release by NK cells upon treatment with the immunoconjugates, particularly immunoconjugates comprising IL-2 as effector moiety. For this purpose, peripheral blood mononuclear cells (PBMCs) were isolated according to standard procedures, using Histopaque-1077 (Sigma Diagnostics Inc., St. Louis, Mo., USA). In brief, venous blood was taken with heparinized syringes from healthy volunteers. The blood was diluted 2:1 with PBS not containing calcium or magnesium and layered on Histopaque-1077. The gradient was centrifuged at 450×g for 30 min at room temperature (RT) without breaks. The interphase containing the PBMCs was collected and washed with PBS in total three times (350×g followed by 300×g for 10 min at RT).

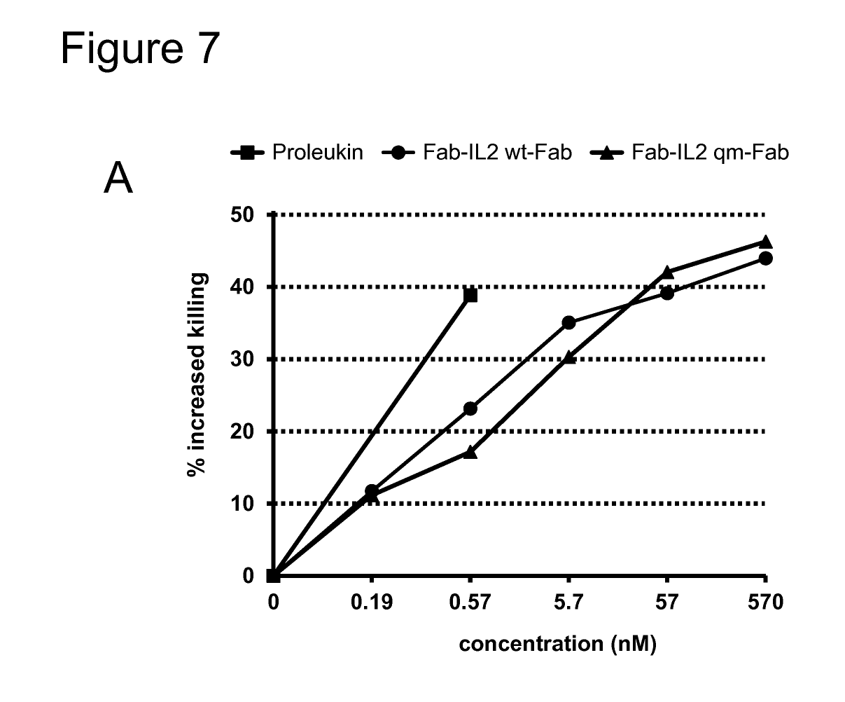

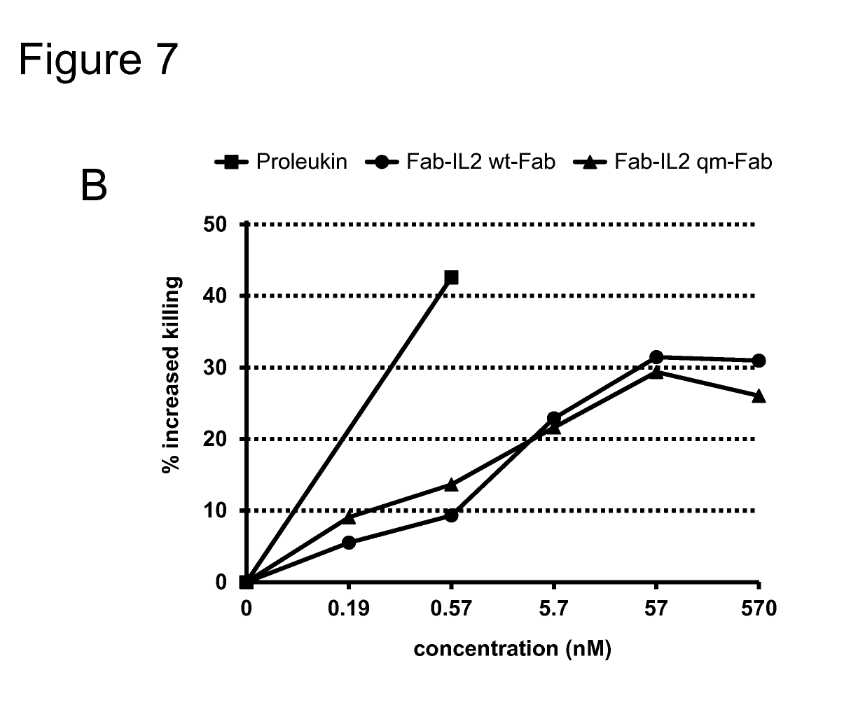

In a first experiment, the isolated PBMCs were incubated with different concentrations of IL-2 (Proleukin) or IL-2 immunoconjugates (FAP-targeted 28H1 Fab-IL2-Fab comprising wildtype or quadruple mutant (qm) IL-2). Two experimental settings were tested; “in solution” in which the IL-2 containing constructs were added to cell supernatants, and “coated” in which the IL-2 containing constructs were bound to FAP, which was previously coated on 96-F-well-plates (500 ng/well in PBS for 20 h at 4° C.). Unbound immunoconjugates were washed away before addition of the PBMCs. In both cases, PBMCs were pre-treated with IL-2 containing constructs for 48 h, then recovered and used for killing of K562 target cells at an effector to target cell ratio (E:T) of 10:1 for 4 h. Target cell killing was detected by measuring LDH release into the cell supernatants (Roche Cytotoxicity Detection Kit LDH). FIG. 7 shows the increase in K562 tumor cell killing upon pre-treatment of the effector cells (PBMCs) with IL-2 constructs in solution (A) or coated to the cell dish (B), compared to untreated PBMCs. IL-2 as well as the Fab-IL2-Fab immunoconjugates boosted the capacity of PBMCs to kill target cells.

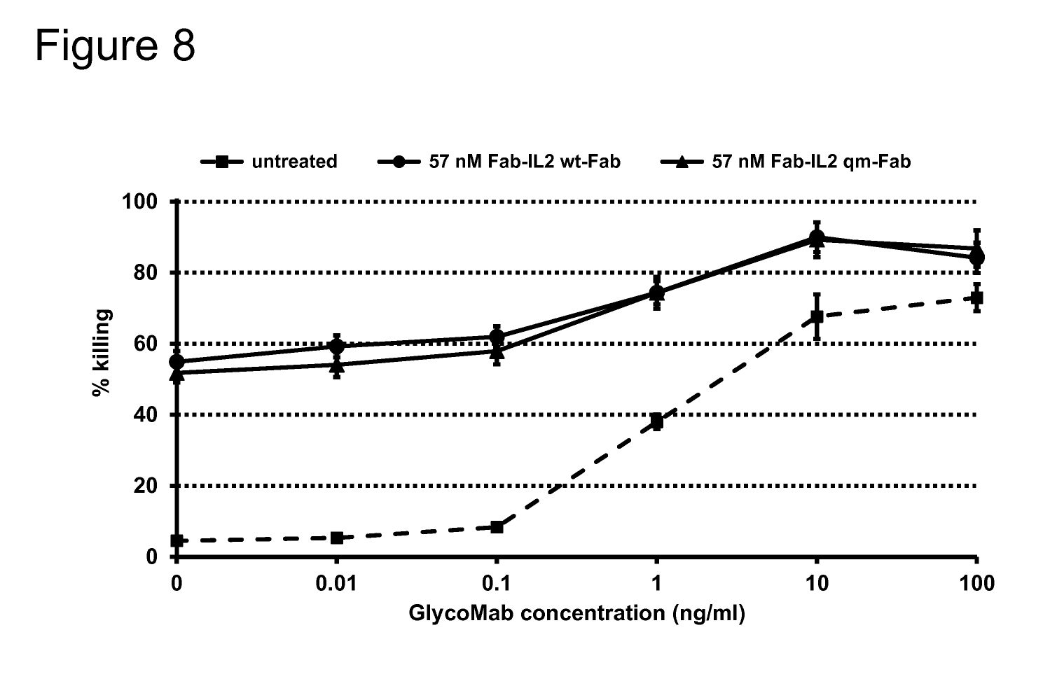

In a second experiment, the isolated PBMCs were incubated with IL-2 (Proleukin) or IL-2 immunoconjugates, added to the cell supernatant, for 45 h. Subsequently, the PBMCs were recovered and used for anti-EGFR GlycoMab-mediated ADCC of A549 cells at an E:T of 10:1, for 4 h. Target cell killing was detected by measuring LDH release into the cell supernatants (Roche Cytotoxicity Detection Kit LDH). FIG. 8 shows the overall A549 tumor cell killing by PBMCs, pre-treated or not with 57 nM FAP-targeted 28H1 Fab-IL2-Fab comprising wildtype (wt) or quadruple mutant (qm) IL-2, in the presence of different concentrations of anti-EGFR GlycoMab. The result shows that nearly 100% target cell killing can be obtained using the combination of the immunoconjugate and the GlycoMab, which is not achieved by either agent alone under the present experimental conditions. The two immunoconjugates comprising either wildtype or quadruple mutant IL-2 are equally potent.

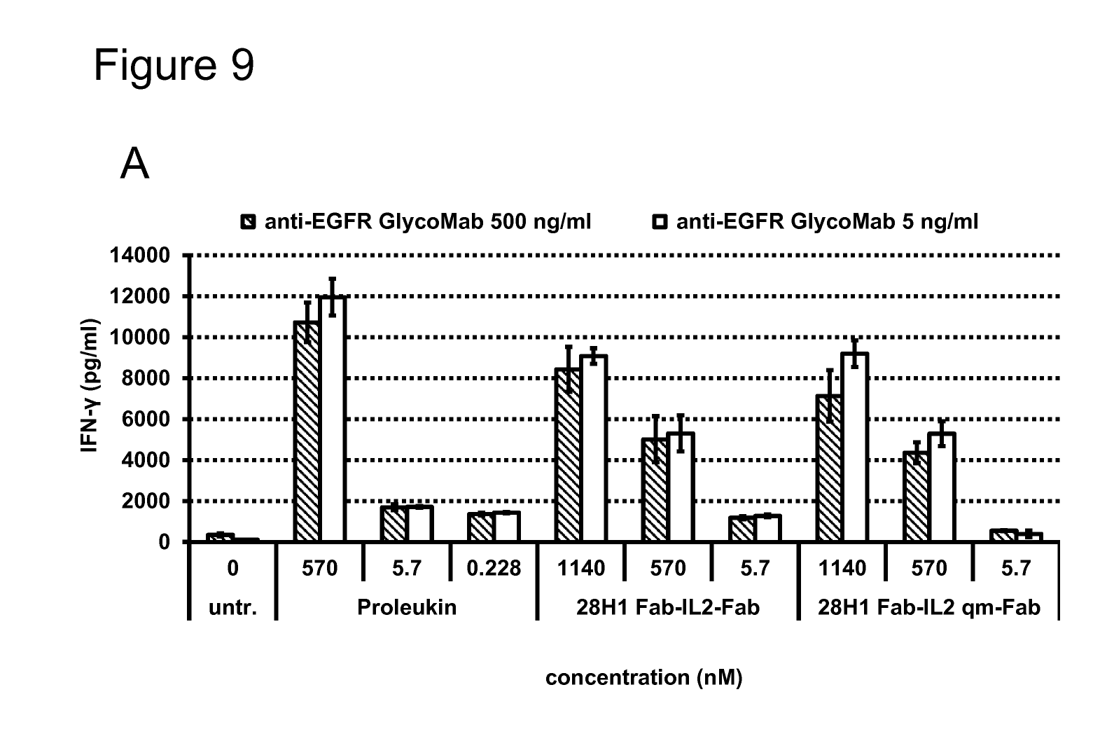

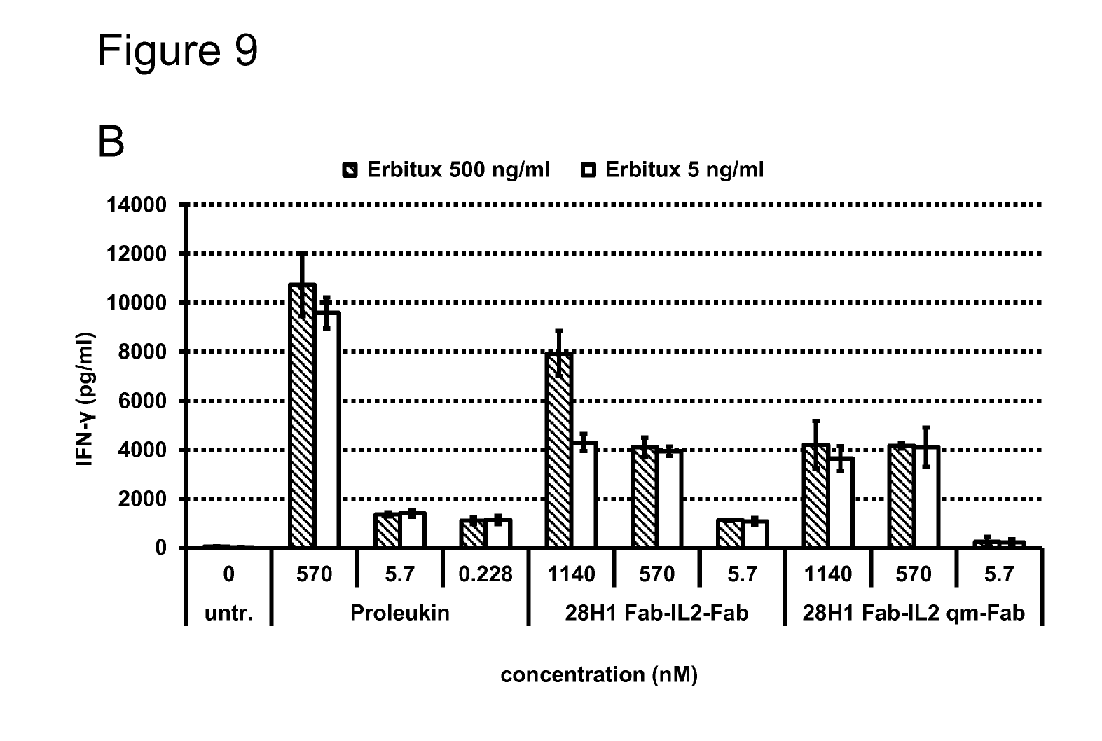

In another experiment, isolated PBMCs were used in an ADCC assay with two different concentrations (5 and 500 ng/ml) of anti-EGFR GlycoMab and a non-glycoengineered anti-EGFR antibody (Erbitux) on A549 cells, at an E:T of 5:1 for 21 h. At the end of the incubation time the release of IFN-γ from PBMCs into the cell supernatant was detected using an IFN-γELISA kit (BD #550612). FIG. 9 shows that, while no significant IFN-γ release was detected after incubation with the antibodies alone, the presence of IL-2 (Proleukin), 28H1 Fab-IL2-Fab or 28H1 Fab-IL2 qm-Fab during the incubation time strongly enhanced IFN-γ release during (A) anti-EGFR GlycoMab- as well as (B) Erbitux-mediated ADCC. Overall, and particularly at the lower antibody concentration (5 ng/ml) and highest IL-2 (immunoconjugate) concentration (1140 nM), IFN-γ release is higher for anti-EGFR GlycoMab than for Erbitux.

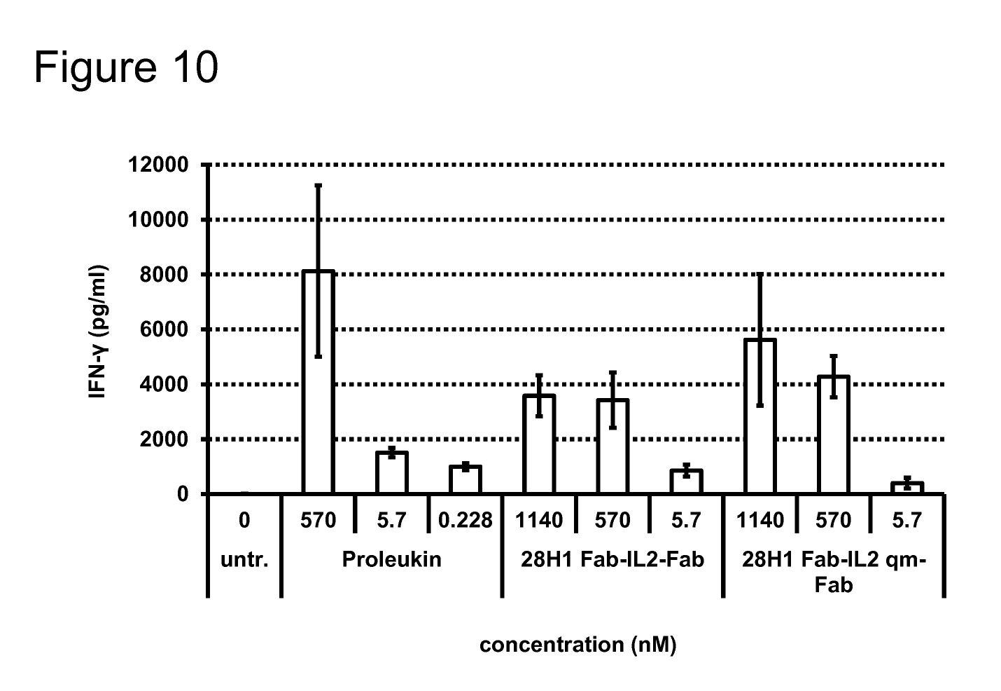

Finally, IFN-γ release from PBMCs after incubation with IL-2 (Proleukin), 28H1 Fab-IL2-Fab or 28H1 Fab-IL2 qm-Fab, but without any antibody, was determined. The experimental conditions were as described above. As shown in FIG. 10, IL-2 (immunoconjugates) enhanced IFN-γ release from PBMCs also in the absence of an ADCC inducing antibody. The IFN-γ levels were comparable to the levels measured in the presence of 5 ng/ml Erbitux (see FIG. 9B), but lower than in the presence of the anti-EGFR GlycoMab (see FIG. 9A).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A combination of (a) an immunoconjugate comprising at least one antigen-binding moiety and an effector moiety, and (b) an antibody engineered to have increased effector function, for use in treating a disease in an individual in need thereof.

2. The combination of claim 1, wherein the effector moiety is a cytokine.

3. The combination of claim 1 or 2, wherein the effector moiety is a cytokine selected from the group consisting of IL-2, GM-CSF, IFN-α, and IL-12.

4. The combination of any one of claims 1 to 3, wherein the effector moiety is IL-2.

5. The combination of claim 4, wherein the IL-2 effector moiety is a mutant IL-2 effector moiety comprising at least one amino acid mutation, particularly an amino acid substitution, that reduces or abolishes the affinity of the mutant IL-2 effector moiety to the α-subunit of the IL-2 receptor but preserves the affinity of the mutant IL-2 effector moiety to the intermediate-affinity IL-2 receptor, compared to the non-mutated IL-2 effector moiety.

6. The combination of any one of claims 1 to 5, wherein the antigen-binding moiety is an antibody or an antibody fragment.

7. The combination of any one of claims 1 to 6, wherein the antigen-binding moiety is selected from a Fab molecule and a scFv molecule.

8. The combination of any one of claims 1 to 7, wherein the immunoconjugate comprises a first and a second antigen-binding moiety.

9. The combination of claim 8, wherein each of said first and said second antigen-binding moieties is a Fab molecule.

10. The combination of claim 8 or 9, wherein the effector moiety shares an amino- or carboxy-terminal peptide bond with the first antigen-binding moiety, and the second antigen-binding moiety shares an amino- or carboxy-terminal peptide bond with either the effector moiety or the first antigen-binding moiety.

11. The combination of any one of claims 1 to 10, wherein the immunoconjugate comprises an effector moiety, particularly a single chain effector moiety, and a first and a second Fab molecule, wherein the effector moiety is joined at its amino-terminal amino acid to the carboxy-terminus of the heavy or light chain of the first Fab molecule, and wherein the effector moiety is joined at its carboxy-terminal amino acid to the amino-terminus of the heavy or light chain of the second Fab molecule.

12. The combination of any one of claims 1 to 11, wherein the antigen-binding moiety is directed to an antigen presented on a tumor cell or in a tumor cell environment.

13. The combination of any one of claims 1 to 12, wherein the antibody engineered to have increased effector function is a full-length IgG class antibody, particularly an IgG1 subclass antibody.

14. The combination of any one of claims 1 to 13, wherein the increased effector function is selected from the group of increased binding to an activating Fc receptor, increased ADCC, increased ADCP, increased CDC, and increased cytokine secretion.

15. The combination of any one of claims 1 to 14, wherein the increased effector function is increased binding to an activating Fc receptor and/or increased ADCC.

16. The combination of any one of claims 1 to 15, wherein the antibody engineered to have increased effector function is engineered by introduction of one or more amino acid mutations in the Fc region or by modification of the glycosylation in the Fc region.

17. The combination of any one of claims 1 to 16, wherein the antibody engineered to have increased effector function is engineered to have an increased proportion of non-fucosylated oligosaccharides in the Fc region as compared to a non-engineered antibody.

18. The combination of any one of claims 1 to 17, wherein the antibody engineered to have increased effector function is directed to an antigen presented on a tumor cell.

19. The combination of any one of claims 1 to 18, wherein the disease is a disorder treatable by stimulation of effector cell function, particularly cancer.

20. The combination of any one of claims 1 to 19, wherein the individual is a mammal, particularly a human.

21. A pharmaceutical composition comprising (a) an immunoconjugate comprising at least one antigen-binding moiety and an effector moiety, and (b) an antibody engineered to have increased effector function, in a pharmaceutically acceptable carrier.

22. Use of (a) an immunoconjugate comprising at least one antigen binding moiety and an effector moiety, and (b) an antibody engineered to have increased effector function, for the manufacture of a medicament for the treatment of a disease in an individual.

23. A method of treating a disease in an individual, comprising administering to the individual a combination of (a) an immunoconjugate comprising at least one antigen binding moiety and an effector moiety, and (b) an antibody engineered to have increased effector function, in a therapeutically effective amount.

24. A method of stimulating effector cell function in an individual, comprising administering to the individual a combination of (a) an immunoconjugate comprising at least one antigen binding moiety and an effector moiety, and (b) an antibody engineered to have increased effector function, in an amount effective to stimulate effector cell function.

25. A kit intended for the treatment of a disease, comprising in the same or in separate containers (a) an immunoconjugate comprising at least one antigen binding moiety and an effector moiety, (b) an antibody engineered to have increased effector function, and (c) optionally a package insert comprising printed instructions directing the use of the combined treatment as a method for treating the disease.

26. The invention as described hereinbefore.


SEQUENCE LISTING (156 sequences)

SEQ ID NO: 1 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2 - protein, 133 aa, Homo sapiens
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 3 - protein, 107 aa, Artificial Sequence
  2B10; VL
DIQMTQSPSSLSASVGDRVTITCRASQGIRNDLGWYQQKPGKAPKRLIYAASSLQSGVPSRFSGGGSGTEFTLTISSLQPEDFATYYCLQNGLQPATFGQGTKVEIK

SEQ ID NO: 4 - protein, 107 aa, Artificial Sequence
  2B10(GS); VL
DIQMTQSPSSLSASVGDRVTITCRASQGIRNDLGWYQQKPGKAPKRLIYAASSLQSGVPSRFSGSGSGTEFTLTISSLQPEDFATYYCLQNGLQPATFGQGTKVEIK

SEQ ID NO: 5 - protein, 121 aa, Artificial Sequence
  2B10; VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLYGYAYYGAFDYWGQGTTVTVSS

SEQ ID NO: 6 - protein, 108 aa, Artificial Sequence
  2F11; VL
EIVLTQSPGTLSLSPGERATLSCRASQSVSSSYLAWYQQKPGQAPRLLIYGASSRATGVPDRFSGSGSGTDFTLTISRLEPEDFAVYYCQQGQYTPPTFGQGTKVEIK

SEQ ID NO: 7 - protein, 108 aa, Artificial Sequence
  2F11(VI); VL
EIVLTQSPGTLSLSPGERATLSCRASQSVSSSYLAWYQQKPGQAPRLLIYGASSRATGIPDRFSGSGSGTDFTLTISRLEPEDFAVYYCQQGQYTPPTFGQGTKVEIK

SEQ ID NO: 8 - protein, 117 aa, Artificial Sequence
  2F11; VH
EVQLLESGGGLVQPGGSLRLSCAASGFTFSSYAMSWVRQAPGKGLEWVSAISGSGGSTYYADSVKGRFTISRDNSKNTLYLQMNSLRAEDMAVYYCAKWRWMMFDYWGQGTLVTVSS

SEQ ID NO: 9 - protein, 117 aa, Artificial Sequence
  2F11(MT); VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWRWMMFDYWGQGTLVTVSS

SEQ ID NO: 10 - protein, 108 aa, Artificial Sequence
  3F2; VL
EIVLTQSPGTLSLYPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 11 - protein, 108 aa, Artificial Sequence
  3F2(YS); VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 12 - protein, 117 aa, Artificial Sequence
  3F2; VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSS

SEQ ID NO: 13 - protein, 108 aa, Artificial Sequence
  3D9, VL
EIVLTQSPGTLSLSPGERATLSCRASQSVSSSYLAWYQQKPGQAPRLLIYGASSRATGIPDRFSGSGSGTDFTLTISRLEPEDFAVYYCQQGQLIPPTFGQGTKVEIK

SEQ ID NO: 14 - protein, 117 aa, Artificial Sequence
  3D9, VH
EVQLLESGGGLVQPGGSLRLSCAASGFTFSSYAMSWVRQTPGKGLEWVSAIGVSTGSTYYADSVKGRFTISRDNSKNTLYLQMNSLRAEDTAVYYCAKGWLGPFDYWGQGTLVTVSS

SEQ ID NO: 15 - protein, 117 aa, Artificial Sequence
  3D9(TA); VH
EVQLLESGGGLVQPGGSLRLSCAASGFTFSSYAMSWVRQAPGKGLEWVSAIGVSTGSTYYADSVKGRFTISRDNSKNTLYLQMNSLRAEDTAVYYCAKGWLGPFDYWGQGTLVTVSS

SEQ ID NO: 16 - protein, 108 aa, Artificial Sequence
  4G8; VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 17 - protein, 117 aa, Artificial Sequence
  4G8; VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSS

SEQ ID NO: 18 - protein, 108 aa, Artificial Sequence
  4B3; VL
EIVLTQSPGTLSLSPGERATLSCRASQSVSSNYLAWYQQKPGQAPRLLIYGAYIRATGIPDRFSGSGSGTDFTLTISRLEPEDFAVYYCQQGQVIPPTFGQGTKVEIK

SEQ ID NO: 19 - protein, 117 aa, Artificial Sequence; identical to SEQ ID NO: 17
  4B3; VH

SEQ ID NO: 20 - protein, 108 aa, Artificial Sequence
  4D6; VL
EIVLTQSPGTLSLSPGERATLSCRASQSVSSNYLAWYQQKPGQAPRLLIQGASSRATGIPDRFSGSGSGTDFTLTISRLEPEDFAVYYCQQGQVIPPTFGQGTKVEIK

SEQ ID NO: 21 - protein, 117 aa, Artificial Sequence; identical to SEQ ID NO: 17
  4D6; VH

SEQ ID NO: 22 - protein, 108 aa, Artificial Sequence
  2C6; VL
EIVLTQSPGTLSLSPGERATLSCRASQSVSSSYLAWYQQKPGQAPRLLIYGASSRATGIPDRFSGSGSGTDFTLTISRLEPEDFAVYYCQQGQQIPPTFGQGTKVEIK

SEQ ID NO: 23 - protein, 117 aa, Artificial Sequence
  2C6; VH
EVQLLESGGGLVQPGGSLRLSCAASGSTFSSYAMSWVRQAPGKGLEWVSAISGSAGYTYYADSVKGRFTISRDNSKNTLYLQMNSLRAEDTAVYYCAKGWFGNFDYWGQGTLVTVSS

SEQ ID NO: 24 - protein, 108 aa, Artificial Sequence
  5H5; VL
EIVLTQSPGTLSLSPGERATLSCRASQSVSSSYLAWYQQKPGQAPRLLIYGASSRATGIPDRFSGSGSGTDFTLTISRLEPEDFAVYYCQQGNQIPPTFGQGTKVEIK

SEQ ID NO: 25 - protein, 116 aa, Artificial Sequence
  5H5; VH
EVQLLESGGGLVQPGGSLRLSCAASGFTFSSYTMSWVRRSPGKGLEWVSAISGGGRTYYADSVKGRFTISRDNSKNTLYLQMNSLRAEDTAVYYCAKGWFTPFDYWGQGTLVTVSS

SEQ ID NO: 26 - protein, 108 aa, Artificial Sequence
  2C4; VL
EIVLTQSPGTLSLSPGERATLSCRASQSVSSNYLAWYQQKPGQAPRLLIYGASIRATGIPDRFSGSGSGTDFTLTISRLEPEDFAVYYCQQGNQIPPTFGQGTKVEIK

SEQ ID NO: 27 - protein, 117 aa, Artificial Sequence
  2C4; VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFTPFDYWGQGTLVTVSS

SEQ ID NO: 28 - protein, 108 aa, Artificial Sequence; identical to SEQ ID NO: 24
  2D9; VL

SEQ ID NO: 29 - protein, 117 aa, Artificial Sequence; identical to SEQ ID NO: 27
  2D9; VH

SEQ ID NO: 30 - protein, 108 aa, Artificial Sequence
  4B8; VL
EIVLTQSPGTLSLSPGERATLSCRASQSVSSSYLAWYQQKPGQAPRLLIYGASSRATGIPDRFSGSGSGTDFTLTISRLEPEDFAVYYCQQGQVIPPTFGQGTKVEIK

SEQ ID NO: 31 - protein, 117 aa, Artificial Sequence; identical to SEQ ID NO: 17
  4B8; VH

SEQ ID NO: 32 - protein, 108 aa, Artificial Sequence; identical to SEQ ID NO: 22
  7A1; VL

SEQ ID NO: 33 - protein, 117 aa, Artificial Sequence
  7A1; VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGNFDYWGQGTLVTVSS

SEQ ID NO: 34 - protein, 108 aa, Artificial Sequence; identical to SEQ ID NO: 13
  13C2; VL

SEQ ID NO: 35 - protein, 117 aa, Artificial Sequence
  13C2; VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGPFDYWGQGTLVTVSS

SEQ ID NO: 36 - protein, 108 aa, Artificial Sequence
  13E8; VL
EIVLTQSPGTLSLSPGERATLSCRASQSVSSSYLAWYQQKPGQAPRLLIYGASSRATGIPDRFSGSGSGTDFTLTISRLEPEDFAVYYCQQGLNIPSTFGQGTKVEIK

SEQ ID NO: 37 - protein, 117 aa, Artificial Sequence; identical to SEQ ID NO: 35
  13E8; VH

SEQ ID NO: 38 - protein, 108 aa, Artificial Sequence
  14C10; VL
EIVLTQSPGTLSLSPGERATLSCRASQSVSSSYLAWYQQKPGQAPRLLIYGASSRATGIPDRFSGSGSGTDFTLTISRLEPEDFAVYYCQQGHIIPPTFGQGTKVEIK

SEQ ID NO: 39 - protein, 117 aa, Artificial Sequence
  14C10; VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAWMGPFDYWGQGTLVTVSS

SEQ ID NO: 40 - protein, 108 aa, Artificial Sequence; identical to SEQ ID NO: 36
  17A11; VL

SEQ ID NO: 41 - protein, 117 aa, Artificial Sequence; identical to SEQ ID NO: 35
  17A11; VH

SEQ ID NO: 42 - protein, 108 aa, Artificial Sequence; identical to SEQ ID NO: 11
  19G1; VL

SEQ ID NO: 43 - protein, 117 aa, Artificial Sequence
  19G1; VH
EVQLLESGGGLVQPGGSLRLSCAASGFTFSSYAMSWVRQAPGKGLEWVSAIISSGGLTYYADSVKGRFTISRDNSKNTLYLQMNSLRAEDTAVYYCAKGWFGGFNYWGQGTLVTVSS

SEQ ID NO: 44 - protein, 108 aa, Artificial Sequence; identical to SEQ ID NO: 11
  20G8; VL

SEQ ID NO: 45 - protein, 117 aa, Artificial Sequence
  20G8; VH
EVQLLESGGGLVQPGGSLRLSCAASGFTFSSYAMSWVRQAPGKGLEWVSAIIGSGSRTYYADSVKGRFTISRDNSKNTLYLQMNSLRAEDTAVYYCAKGWFGGFNYWGQGTLVTVSS

SEQ ID NO: 46 - protein, 108 aa, Artificial Sequence; identical to SEQ ID NO: 11
  4B9; VL

SEQ ID NO: 47 - protein, 117 aa, Artificial Sequence
  4B9; VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 48 - protein, 108 aa, Artificial Sequence; identical to SEQ ID NO: 11
  5B8; VL

SEQ ID NO: 49 - protein, 117 aa, Artificial Sequence
  5B8; VH
EVQLLESGGGLVQPGGSLRLSCAASGFTFSSYAMSWVRQAPGKGLEWVSAIWGGGRSTYYADSVKGRFTISRDNSKNTLYLQMNSLRAEDTAVYYCAKGWFGGFNYWGQGTLVTVSS

SEQ ID NO: 50 - protein, 108 aa, Artificial Sequence; identical to SEQ ID NO: 11
  5F1; VL

SEQ ID NO: 51 - protein, 117 aa, Artificial Sequence
  5F1; VH
EVQLLESGGGLVQPGGSLRLSCAASGFTFSSYAMSWVRQAPGKGLEWVSAIISSGASTYYADSVKGRFTISRDNSKNTLYLQMNSLRAEDTAVYYCAKGWFGGFNYWGQGTLVTVSS

SEQ ID NO: 52 - protein, 108 aa, Artificial Sequence; identical to SEQ ID NO: 11
  14B3; VL

SEQ ID NO: 53 - protein, 117 aa, Artificial Sequence
  14B3; VH
EVQLLESGGGLVQPGGSLRLSCAASGFTFSSYAMSWVRQAPGKGLEWVSAILASGAITYYADSVKGRFTISRDNSKNTLYLQMNSLRAEDTAVYYCAKGWFGGFNYWGQGTLVTVSS

SEQ ID NO: 54 - protein, 108 aa, Artificial Sequence; identical to SEQ ID NO: 11
  16F1; VL

SEQ ID NO: 55 - protein, 117 aa, Artificial Sequence
  16F1; VH
EVQLLESGGGLVQPGGSLRLSCAASGFTFSSYAMSWVRQAPGKGLEWVSGIIGSGGITYYADSVKGRFTISRDNSKNTLYLQMNSLRAEDTAVYYCAKGWFGGFNYWGQGTLVTVSS

SEQ ID NO: 56 - protein, 108 aa, Artificial Sequence; identical to SEQ ID NO: 11
  16F8; VL

SEQ ID NO: 57 - protein, 117 aa, Artificial Sequence
  16F8; VH
EVQLLESGGGLVQPGGSLRLSCAASGFTFSSYAMSWVRQAPGKGLEWVSAILGSGGSTYYADSVKGRFTISRDNSKNTLYLQMNSLRAEDTAVYYCAKGWFGGFNYWGQGTLVTVSS

SEQ ID NO: 58 - protein, 108 aa, Artificial Sequence; identical to SEQ ID NO: 11
  O3C9; VL

SEQ ID NO: 59 - protein, 117 aa, Artificial Sequence
  O3C9; VH
EVQLLESGGGLVQPGGSLRLSCAASGFTFSSFAMSWVRQSPGKGLEWVSAIIGSGSNTYYADSVKGRFTISRDNSKNTLYLQMNSLRAEDTAVYYCAKGWFGGFNYWGQGTLVTVSS

SEQ ID NO: 60 - protein, 108 aa, Artificial Sequence
  O2D7; VL
EIVLTQSPGTLSLSPGERATLSCRASQSVTSSYLAWYQQKPGQAPRLLINVGSRRATGTPDRFSGSGSGTDFTLTISRLEPEDFAVYYCQQAIMLPPTFGQGTKVEIK

SEQ ID NO: 61 - protein, 117 aa, Artificial Sequence; identical to SEQ ID NO: 12
  O2D7; VH

SEQ ID NO: 62 - protein, 108 aa, Artificial Sequence; identical to SEQ ID NO: 16
  28H1; VL

SEQ ID NO: 63 - protein, 116 aa, Artificial Sequence
  28H1; VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 64 - protein, 108 aa, Artificial Sequence; identical to SEQ ID NO: 11
  22A3; VL

SEQ ID NO: 65 - protein, 117 aa, Artificial Sequence
  22A3; VH
EVQLLESGGGLVQPGGSLRLSCAASGFTFSSYAMSWVRQAPGKGLEWVSAIIGSGSITYYADSVKGRFTISRDNSKNTLYLQMNSLRAEDTAVYYCAKGWFGGFNYWGQGTLVTVSS

SEQ ID NO: 66 - protein, 108 aa, Artificial Sequence; identical to SEQ ID NO: 11
  29B11; VL

SEQ ID NO: 67 - protein, 117 aa, Artificial Sequence
  29B11; VH
EVQLLESGGGLVQPGGSLRLSCAASGFTFSSYAMSWVRQAPGKGLEWVSAIIGSGGITYYADSVKGRFTISRDNSKNTLYLQMNSLRAEDTAVYYCAKGWFGGFNYWGQGTLVTVSS

SEQ ID NO: 68 - protein, 108 aa, Artificial Sequence; identical to SEQ ID NO: 16
  23C10; VL

SEQ ID NO: 69 - protein, 117 aa, Artificial Sequence
  23C10; VH
EVQLLESGGGLVQPGGSLRLSCAASGFTFSSSAMSWVRQAPGKGLEWVSAISTNGNYTYYADSVKGRFTISRDNSKNTLYLQMNSLRAEDTAVYYCAKGWLGNFDYWGQGTLVTVSS

SEQ ID NO: 70 - protein, 107 aa, Artificial Sequence; identical to SEQ ID NO: 4
  2B10_C3B6; VL

SEQ ID NO: 71 - protein, 121 aa, Artificial Sequence
  2B10_C3B6; VH
QVQLVQSGAEVKKPGSSVKVSCKASGGTFSSYAISWVRQAPGQGLEWMGAIIPILGIANYAQKFQGRVTITADKSTSTAYMELSSLRSEDTAVYYCARLYGYAYYGAFDYWGQGTTVTVSS

SEQ ID NO: 72 - protein, 107 aa, Artificial Sequence; identical to SEQ ID NO: 4
  2B10_6A12; VL

SEQ ID NO: 73 - protein, 121 aa, Artificial Sequence
  2B10_6A12; VH
QVQLVQSGAEVKKPGSSVKVSCKASGGTFSSYAISWVRQAPGQGLEWMGVIIPILGTANYAQKFQGRVTITADKSTSTAYMELSSLRSEDTAVYYCARLYGYAYYGAFDYWGQGTTVTVSS

SEQ ID NO: 74 - protein, 107 aa, Artificial Sequence
  2B10_C3A6; VL
DIQMTQSPSSLSASVGDRVTITCRASQGIRNVLGWYQQKPGKAPKRLIYDSSSLQSGVPSRFSGGGSGTEFTLTISSLQPEDFATYYCLQNGLQPATFGQGTKVEIK

SEQ ID NO: 75 - protein, 121 aa, Artificial Sequence; identical to SEQ ID NO: 5
  2B10_C3A6; VH

SEQ ID NO: 76 - protein, 107 aa, Artificial Sequence
  2B10_D1A2_wt; VL
DIQMTQSPSSLSASVGDRVTITCRASQGIRNVLGWYQQKPGKAPKRLIYDAYSLQSGVPSRFSGGGSGTEFTLTISSLQPEDFATYYCLQNGLQPATFGQGTKVEIK

SEQ ID NO: 77 - protein, 121 aa, Artificial Sequence; identical to SEQ ID NO: 5
  2B10_D1A2_wt; VH

SEQ ID NO: 78 - protein, 107 aa, Artificial Sequence
  2B10_D1A2_VD; VL
DIQMTQSPSSLSASVGDRVTITCRASQGIRNDLGWYQQKPGKAPKRLIYDAYSLQSGVPSRFSGGGSGTEFTLTISSLQPEDFATYYCLQNGLQPATFGQGTKVEIK

SEQ ID NO: 79 - protein, 121 aa, Artificial Sequence; identical to SEQ ID NO: 5
  2B10_D1A2_VD; VH

SEQ ID NO: 80 - protein, 107 aa, Artificial Sequence
  2B10_O7D8; VL
DIQMTQSPSSLSASVGDRVTITCRASQSIRNVLGWYQQKPGKAPKRLIYDVSSLQSGVPSRFSGGGSGTEFTLTISSLQPEDFATYYCLQNGLQPATFGQGTKVEIK

SEQ ID NO: 81 - protein, 121 aa, Artificial Sequence; identical to SEQ ID NO: 5
  2B10_O7D8; VH

SEQ ID NO: 82 - protein, 107 aa, Artificial Sequence
  2B10_O1F7; VL
DIQMTQSPSSLSASVGDRVTITCRASQGIRNVLGWYQQKPGKAPKRLIYDASSLQSGVPSRFSGGGSGTEFTLTISSLQPEDFATYYCLQNGLQPATFGQGTKVEIK

SEQ ID NO: 83 - protein, 121 aa, Artificial Sequence; identical to SEQ ID NO: 5
  2B10_O1F7; VH

SEQ ID NO: 84 - protein, 107 aa, Artificial Sequence
  2B10_6H10; VL
DIQMTQSPSSLSASVGDRVTITCRASQGIRNVLGWYQQKPGKAPKRLIQAATSLQSGVPSRFSGGGSGTEFTLTISSLQPEDFATYYCLQNGLQPATFGQGTKVEIK

SEQ ID NO: 85 - protein, 121 aa, Artificial Sequence; identical to SEQ ID NO: 5
  2B10_6H10; VH

SEQ ID NO: 86 - protein, 122 aa, Artificial Sequence
  MHLG1; VH
EVQLVESGGGLVKPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSS

SEQ ID NO: 87 - protein, 109 aa, Artificial Sequence
  KV9; VL
DIQLTQSPSFLSASVGDRVTITCKASQNVDTNVAWYQQKPGQAPRPLIYSASYRYTGVPSRFSGSGSGTEFTLTISSLQPEDFATYYCQQYNSYPLTFGGGTKVEIKRT

SEQ ID NO: 88 - protein, 122 aa, Artificial Sequence
  MHLG; VH
EVQLVESGGGLVQPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSS

SEQ ID NO: 89 - protein, 109 aa, Artificial Sequence
  KV1; VL
DIQLTQSPSFLSASVGDRVTITCRASQNVDTNLAWYQQKPGKAPKLLIYSASYRYTGVPSRFSGSGSGTEFTLTISSLQPEDFATYYCQQYNSYPLTFGGGTKVEIKRT

SEQ ID NO: 90 - protein, 109 aa, Artificial Sequence
  K7; VL
DIQLTQSPSFLSASVGDRVTITCKASQNVDTNVAWYQQKPGKAPKPLIYSASYRYTGVPSRFSGSGSGTEFTLTISSLQPEDFATYYCQQYNSYPLTFGGGTKVEIKRT

SEQ ID NO: 91 - protein, 603 aa, Artificial Sequence [sequence omitted]

SEQ ID NO: 92 - protein, 215 aa, Artificial Sequence
  Fab light chain derived from L19 monoclonal antibody
EIVLTQSPGTLSLSPGERATLSCRASQSVSSSYLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 382 aa, Artificial Sequence
  scFv derived from L19 monoclonal antibody-8 amino acid linker-C125A variant of IL2
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSSGGSGGASEIVLTQSPGTLSLSPGERATLSCRASQSVSSSYLAWYQQKPGQAPRLLIYYASSRATGIPDRFSGSGSGTDFTLTISRLEPEDFAVYYCQQTGRIPPTFGQGTKVEISVLSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 94 - protein, 377 aa, Artificial Sequence
  F16-diabody-IL2 protein
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSSASGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLGSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 95 - protein, 641 aa, Artificial Sequence
  scFv-IL2-scFv (F16, protein)
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLGSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLGSGGGSGGGSGGGSGSEVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVS

SEQ ID NO: 96 - protein, 603 aa, Artificial Sequence [sequence omitted]

SEQ ID NO: 97 - protein, 214 aa, Artificial Sequence
  F16, light chain, protein
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 98 - protein, 991 aa, Artificial Sequence
  Fab-IL12-Fab, L19 antibody, murine scIL12, protein
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPRSYFSLKFFVRIQRKKEKMKETEEGCNQKGAFFVEKTSTEVQCKGGNVCVQAQDRYYNSSCSKWACVPCRVRSGGDGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 99 - protein, 987 aa, Artificial Sequence [sequence omitted]

SEQ ID NO: 100 - protein, 597 aa, Artificial Sequence [sequence omitted]

SEQ ID NO: 101 - protein, 636 aa, Artificial Sequence
  Fab-IFNa2-Fab, L19 antibody, protein
EVQLLESGGGLVQPGGSLRLSCAASGFTFSSFSMSWVRQAPGKGLEWVSSIRGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKESGGGGSGGGGSGGGGEVQLLESGGGLVQPGGSLRLSCAASGFTFSSFSMSWVRQAPGKGLEWVSSIRGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 102 - protein, 605 aa, Artificial Sequence [sequence omitted]

SEQ ID NO: 103 - protein, 605 aa, Artificial Sequence [sequence omitted]

SEQ ID NO: 104 - protein, 605 aa, Artificial Sequence
  3D9 Fab-IL2-Fab (heavy chain cytokine fusion construct)
EVQLLESGGGLVQPGGSLRLSCAASGFTFSSYAMSWVRQTPGKGLEWVSAIGVSTGSTYYADSVKGRFTISRDNSKNTLYLQMNSLRAEDTAVYYCAKGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGEVQLLESGGGLVQPGGSLRLSCAASGFTFSSYAMSWVRQTPGKGLEWVSAIGVSTGSTYYADSVKGRFTISRDNSKNTLYLQMNSLRAEDTAVYYCAKGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 105 - protein, 605 aa, Artificial Sequence
  2F11 Fab-IL2-Fab (heavy chain cytokine fusion construct)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWRWMMFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWRWMMFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 106 - protein, 605 aa, Artificial Sequence [sequence omitted]

SEQ ID NO: 107 - protein, 993 aa, Artificial Sequence
  4G8 Fab-IL12-Fab (murine IL-12; heavy chain cytokine fusion construct)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSPPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPRSYFSLKFFVRIQRKKEKMKETEEGCNQKGAFFVEKTSTEVQCKGGNVCVQAQDRYYNSSCSKWACVPCRVRSGGDGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 108 - protein, 603 aa, Artificial Sequence
  28H1 Fab-IL2-Fab (heavy chain cytokine fusion construct)
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 109 - protein, 605 aa, Artificial Sequence [sequence omitted]

SEQ ID NO: 110 - protein, 605 aa, Artificial Sequence
  19G1 Fab-IL2-Fab (heavy chain cytokine fusion construct)
EVQLLESGGGLVQPGGSLRLSCAASGFTFSSYAMSWVRQAPGKGLEWVSAIISSGGLTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGEVQLLESGGGLVQPGGSLRLSCAASGFTFSSYAMSWVRQAPGKGLEWVSAIISSGGLTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 111 - protein, 605 aa, Artificial Sequence [sequence omitted]

SEQ ID NO: 112 - protein, 215 aa, Artificial Sequence
  3F2 light chain
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 215 aa, Artificial Sequence
  4G8 light chain
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 215 aa, Artificial Sequence
  3D9 light chain
EIVLTQSPGTLSLSPGERATLSCRASQSVSSSYLAWYQQKPGQAPRLLIYGASSRATGIPDRFSGSGSGTDFTLTISRLEPEDFAVYYCQQGQLIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, Artificial Sequence
  2F11 light chain
EIVLTQSPGTLSLSPGERATLSCRASQSVSSSYLAWYQQKPGQAPRLLIYGASSRATGIPDRFSGSGSGTDFTLTISRLEPEDFAVYYCQQGQYTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, Artificial Sequence
  4B3 light chain
EIVLTQSPGTLSLSPGERATLSCRASQSVSSNYLAWYQQKPGQAPRLLIYGAYIRATGIPDRFSGSGSGTDFTLTISRLEPEDFAVYYCQQGQVIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 613 aa, Artificial Sequence [sequence omitted]

SEQ ID NO: 118 - protein, 613 aa, Artificial Sequence [sequence omitted]

SEQ ID NO: 119 - protein, 613 aa, Artificial Sequence [sequence omitted]

SEQ ID NO: 120 - protein, 214 aa, Artificial Sequence
  2B10 light chain
DIQMTQSPSSLSASVGDRVTITCRASQGIRNDLGWYQQKPGKAPKRLIYAASSLQSGVPSRFSGSGSGTEFTLTISSLQPEDFATYYCLQNGLQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 214 aa, Artificial Sequence
  D1A2 light chain
DIQMTQSPSSLSASVGDRVTITCRASQGIRNDLGWYQQKPGKAPKRLIYDAYSLQSGVPSRFSGGGSGTEFTLTISSLQPEDFATYYCLQNGLQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, Artificial Sequence
  O7D8 light chain
DIQMTQSPSSLSASVGDRVTITCRASQSIRNVLGWYQQKPGKAPKRLIYDVSSLQSGVPSRFSGGGSGTEFTLTISSLQPEDFATYYCLQNGLQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 615 aa, Artificial Sequence
  MHLG1 Fab-IL2-Fab (heavy chain cytokine fusion construct)
EVQLVESGGGLVKPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGEVQLVESGGGLVKPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 124 - protein, 214 aa, Artificial Sequence
  KV9 light chain
DIQLTQSPSFLSASVGDRVTITCKASQNVDTNVAWYQQKPGQAPRPLIYSASYRYTGVPSRFSGSGSGTEFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 615 aa, Artificial Sequence
  MHLG Fab-IL2-Fab (heavy chain cytokine fusion construct)
EVQLVESGGGLVQPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDSGGGGSGGGGSGGGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTSGGGGSGGGGSGGGGEVQLVESGGGLVQPGGSLRLSCAASGFTFSNYWMNWVRQAPGKGLEWVAEIRLKSNNFGRYYAASVKGRFTISRDDSKNTLYLQMNSLKTEDTAVYYCTTYGNYVGHYFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 126 - protein, 214 aa, Artificial Sequence
  KV1 light chain
DIQLTQSPSFLSASVGDRVTITCRASQNVDTNLAWYQQKPGKAPKLLIYSASYRYTGVPSRFSGSGSGTEFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 214 aa, Artificial Sequence
  KV7 light chain
DIQLTQSPSFLSASVGDRVTITCKASQNVDTNVAWYQQKPGKAPKPLIYSASYRYTGVPSRFSGSGSGTEFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 6 aa, Artificial Sequence
  anti-CD20 HCDR1
GYAFSY

SEQ ID NO: 129 - protein, 8 aa, Artificial Sequence
  anti-CD20 HCDR2
FPGDGDTD

SEQ ID NO: 130 - protein, 10 aa, Artificial Sequence
  anti-CD20 HCDR3
NVFDGYWLVY

SEQ ID NO: 131 - protein, 16 aa, Artificial Sequence
  anti-CD20 LCDR1
RSSKSLLHSNGITYLY

SEQ ID NO: 132 - protein, 7 aa, Artificial Sequence
  anti-CD20 LCDR2
QMSNLVS

SEQ ID NO: 133 - protein, 9 aa, Artificial Sequence
  anti-CD20 LCDR3
AQNLELPYT

SEQ ID NO: 134 - protein, 119 aa, Artificial Sequence
  anti-CD20 VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 135 - protein, 115 aa, Artificial Sequence
  anti-CD20 VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 136 - protein, 5 aa, Artificial Sequence
  anti-EGFR HCDR1
DYKIH

SEQ ID NO: 137 - protein, 17 aa, Artificial Sequence
  anti-EGFR HCDR2
YFNPNSGYSTYAQKFQG

SEQ ID NO: 138 - protein, 11 aa, Artificial Sequence
  anti-EGFR HCDR3
LSPGGYYVMDA

SEQ ID NO: 139 - protein, 11 aa, Artificial Sequence
  anti-EGFR LCDR1
RASQGINNYLN

SEQ ID NO: 140 - protein, 7 aa, Artificial Sequence
  anti-EGFR LCDR2
NTNNLQT

SEQ ID NO: 141 - protein, 8 aa, Artificial Sequence
  anti-EGFR LCDR3
LQHNSFPT

SEQ ID NO: 142 - protein, 120 aa, Artificial Sequence [sequence omitted]

SEQ ID NO: 143 - protein, 108 aa, Artificial Sequence
  anti-EGFR VL
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 144 - protein, 118 aa, Artificial Sequence
  anti-IGF-1R VH (1)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 145 - protein, 108 aa, Artificial Sequence
  anti-IGF-1R VL (1)
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 146 - protein, 118 aa, Artificial Sequence
  anti-IGF-1R VH (2)
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 147 - protein, 108 aa, Artificial Sequence
  anti-IGF-1R VL (2)
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

SEQ ID NO: 148 - protein, 5 aa, Artificial Sequence
  anti-CEA HCDR1
EFGMN

SEQ ID NO: 149 - protein, 17 aa, Artificial Sequence
  anti-CEA HCDR2
WINTKTGEATYVEEFKG

SEQ ID NO: 150 - protein, 12 aa, Artificial Sequence
  anti-CEA HCDR3
WDFYDYVEAMDY

SEQ ID NO: 151 - protein, 11 aa, Artificial Sequence
  anti-CEA LCDR1
KASQNVGTNVA

SEQ ID NO: 152 - protein, 7 aa, Artificial Sequence
  anti-CEA LCDR2
SASYRYS

SEQ ID NO: 153 - protein, 10 aa, Artificial Sequence
  anti-CEA LCDR3
HQYYTYPLFT

SEQ ID NO: 154 - protein, 121 aa, Artificial Sequence
  anti-CEA VH
QVQLVQSGSELKKPGASVKVSCKASGYTFTEFGMNWVRQAPGQGLEWMGWINTKTGEATYVEEFKGRFVFSLDTSVSTAYLQISSLKAEDTAVYYCARWDFYDYVEAMDYWGQGTTVTVSS

SEQ ID NO: 155 - protein, 108 aa, Artificial Sequence
  anti-CEA VL
DIQMTQSPSSLSASVGDRVTITCKASQNVGTNVAWYQQKPGKAPKLLIYSASYRYSGVPSRFSGSGSGTDFTLTISSLQPEDFATYYCHQYYTYPLFTFGQGTKLEIK

SEQ ID NO: 156 - protein, 328 aa, Homo sapiens
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK